(19) United States
(12) Patent Application Publication
(10) Pub. No.: US 2026/0241002 A1
(43) Pub. Date: Aug. 20, 2026

(54) THERAPEUTIC TARGETING OF TUMOR ESCAPE BY CAR-MEDIATED TROGOCYTOSIS

(71) Applicant: University of Maryland, Baltimore, Baltimore, MD (US)

(72) Inventors: Tim LUETKENS, Baltimore, MD (US); Kenneth A. DIETZE, Baltimore, MD (US)

(73) Assignee: University of Maryland, Baltimore, Baltimore, MD (US)

(21) Appl. No.: 19/665,739

(22) Filed: May 1, 2026

Related U.S. Application Data
(63) Continuation-in-part of application No. PCT/US2024/053867, filed on Oct. 31, 2024.
(60) Provisional application No. 63/595,007, filed on Nov. 1, 2023.

(51) Int. Cl. A61K 40/42; A61K 40/11; A61K 40/31; A61P 35/00; C07K 14/81; C07K 16/28; C12N 5/0783; G01N 21/76; G01N 33/50

(52) U.S. Cl. CPC A61K 40/4247; A61K 40/11; A61K 40/31; A61P 35/00; C07K 14/8139; C07K 16/2803; C12N 5/0636; G01N 21/76; G01N 33/502; G01N 33/505; C07K 2317/622; C12N 2510/00

(57) ABSTRACT

Populations of CAR T cells that exhibit reduced trogocytosis are disclosed, as well as methods for making such cells and methods of using such cells in the treatment of cancer. A reduction in trogocytosis is achieved by inhibiting Cathepsin B in the CAR T cells and/or inducing ubiquitylation of cancer antigens taken up by the CAR T cells.

### SEQUENCE LISTING

A sequence listing in electronic (XML file) format is filed with this application and incorporated herein by reference. The name of the XML file is “Sequence_listing_0476A.xml”; the file was created on Apr. 30, 2026; the size of the file is 53,092 bytes.

### BACKGROUND

In 2020, there were nearly 20 million new cases of cancer diagnosed and close to 10 million cancer-related deaths worldwide [1], highlighting the need for improved therapeutics. Chimeric Antigen Receptor (CAR) T cell therapy is a highly effective treatment for cancer patients in which patient T cells are genetically modified to express a CAR targeting an antigen expressed on the surface of cancer cells [2]. Current FDA-approved CAR T cells are composed of a single chain variable fragment (scFv)—which acts as a highly specific binding domain—a flexible hinge region, a transmembrane domain, a CD3ζ (domain, and a costimulatory domain. CAR T cells function by specifically recognizing tumor-associated surface antigens via their scFv [8], resulting in T cell activation via the CD3ζ (and costimulatory domains [8]. These CAR T cell products are produced via genetic engineering of a patient's own T cells [8,9], in which peripheral blood mononuclear cells (PBMCs) are first isolated via leukapheresis. Subsequently, T cells are transduced with a gene encoding the CAR, typically via lentiviral or gammaretroviral transduction [8,9]. T cells are then expanded and infused back into the patient. CAR T cells have shown clinical promise in some hematological malignancies, leading to the FDA approval of six CAR T cell products [10]. However, the majority of patients treated with CAR T cells eventually relapse [11,12]. Therefore, it is imperative to develop more effective CAR T cell approaches that minimize the occurrence of relapse in patients.

Antigen loss and a lack of CAR T cell persistence are among the major shortcomings of CAR T cell therapy, often resulting in CAR T cell-resistant relapse [13]. Downregulation of CD19 expression has been shown to lead to tumor cell evasion of CD19 CAR T cells [13]. Similar findings have been shown in BCMA-targeting CAR T cells used in the treatment of multiple myeloma, where BCMA loss or downregulation and a lack of CAR T cell persistence have contributed to relapse and resistance to CAR T cell therapy in patients [14]. Of note, it has been shown that CAR T cells, particularly high affinity CAR T cells, can strip target antigen and plasma membrane from the surface of tumor cells, resulting in the emergence of antigen-negative tumor cells [5], a process known as trogocytosis [15]. Additionally, transfer of target antigen to the surface of CAR T cells has been shown to lead to the killing of such CAR T cells by other CAR T cells in a process known as fratricide [5-7], which may contribute to reduced CAR T cell persistence in patients.

An improved understanding of CAR-mediated trogocytosis (CMT) could lead to the development of therapeutic means for blocking both CMT and CAR T cell fratricide, thus improving progression free survival and overall survival in cancer patients being treated with CAR T cell therapy. For example, limiting CAR T cell-mediated trogocytosis may reduce both the emergence of antigen-negative tumor cells and CAR T cell fratricide, ultimately improving the long-term antitumor activity of CAR T cells.

### BRIEF SUMMARY

As provided herein, the present invention stems from the development of a Luciferase Complementation (CompLuc) Assay by the inventors that allows the process of trogocytosis to be studied in real-time. In conjunction with a flow cytometric analysis of CAR T cells, the inventors used the CompLuc assay to demonstrate trogocytosis can be inhibited in CAR T cells. Such inhibition can ameliorate immune escape due to cancer cell antigen loss and reduce fratricide due to inappropriate presence of cancer cell antigens on the surface of CAR T cells.

These discoveries allow for the production of new or renewed populations of CAR T cells that exhibit reduced trogocytosis. Indeed, the invention may be used to rescue existing CAR T cell lines for continued use in patients having cancers that are resistant to particular CAR T cell treatments. Thus, the present invention is generally directed to CAR T cells that exhibit reduced trogocytosis and to methods of treating cancer using these CAR T cells.

In a first embodiment, the invention can be generally defined as populations of CAR T cells, e.g. T cells expressing chimeric antigen receptors (CAR), that exhibit reduced trogocytosis when in contact with a target cell, such as a cancer cell, expressing the CAR-specific antigen.

In one aspect of this embodiment, the invention is directed to populations of CAR T cells that exhibit reduced trogocytosis, wherein the CAR T cells express or overexpress an inhibitor of Cathepsin B. The inhibitor may be, but is not limited to, a protein, a peptide, antisense RNA, an siRNA, and a small molecule inhibitor.

In a particular aspect, the populations of CAR T cells that exhibit reduced trogocytosis are populations of CAR T cells that overexpress Cystatin A or a functional fragment of Cystatin A. Functional fragments of Cystatin A include, but are not limited to, Cystatin A₁₋₅₇.

In a related aspect, the populations of CAR T cells that exhibit reduced trogocytosis are populations of CAR T cells that overexpress Cystatin B or a functional fragment of Cystatin B.

In certain aspects, the Cathepsin B inhibitor is Ca-074, Ca-074-Me, Cystatin A, Cystatin A₁₋₅₇, Cystatin B, or E-64D.

In another aspect of this embodiment, the invention is directed to populations of CAR T cells that exhibit reduced surface expression of cancer antigens, wherein the CAR T cells express or overexpress effectors of ubiquitylation to promote degradation of cancer antigens transferred to CAR T cells.

Effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

In another aspect of this embodiment, the invention is directed to populations of CAR T cells that (i) exhibit reduced trogocytosis when in contact with a target cell, such as a cancer cell, and (ii) exhibit reduced surface expression of cancer antigens.

In certain aspects, these populations of CAR T cells (i) express or overexpress an inhibitor of Cathepsin B and (ii) express or overexpress effectors of ubiquitylation to promote degradation of cancer antigens transferred to CAR T cells.

The inhibitor of Cathepsin B may be, but is not limited to, a protein, a peptide, antisense RNA, an siRNA, and a small molecule inhibitor.

In a particular aspect, these populations of CAR T cells overexpress Cystatin A or a functional fragment of Cystatin A. Functional fragments of Cystatin A include, but are not limited to, Cystatin A₁₋₅₇.

In a related aspect, these populations of CAR T cells overexpress Cystatin B or a functional fragment of Cystatin B.

In certain aspects, the Cathepsin B inhibitor is Ca-074, Ca-074-Me, Cystatin A, Cystatin A₁₋₅₇, Cystatin B, or E-64D.

In a particular aspect, these populations of CAR T cells that exhibit reduced surface expression of cancer antigens express or overexpress effectors of ubiquitylation to promote degradation of cancer antigens transferred to CAR T cells.

Effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

In a second embodiment, the invention can be generally defined as methods of treating cancer using CAR T cells by inhibiting trogocytosis associated with CAR T cell therapy. Non-limiting examples of means for achieving inhibition of trogocytosis include the following. First, populations of CAR T cells can be treated with an inhibitor of trogocytosis in vitro prior to administration to a subject having cancer. Second, an inhibitor of trogocytosis can be administered to a subject being treating by CAR T cell therapy. Third, populations of CAR T cells can be genetically modified to express or overexpress an inhibitor of trogocytosis prior to administration of the cells to a subject.

Thus, in one aspect of this embodiment, the invention is directed to methods of treating cancer in a subject comprising (a) contacting a population of CAR T cells with a Cathepsin B inhibitor and (b) administering the cells of (a) to a subject having cancer.

In another aspect of this embodiment, the invention is directed to methods of treating cancer in a subject comprising administering a Cathepsin B inhibitor and a population of CAR T cells to a subject having cancer.

In a related aspect, the invention is directed to methods of augmenting anti-cancer activity of CAR T cells in a subject comprising administering a Cathepsin B inhibitor to a subject undergoing CAR T cell therapy.

In another related aspect, the invention is directed to methods of inhibiting trogocytosis in CAR T cells in a subject comprising administering a Cathepsin B inhibitor to a subject undergoing CAR T cell therapy.

In another aspect of this embodiment, the invention is directed to methods of treating cancer in a subject comprising administering a population of CAR T cells genetically modified to express or overexpress a Cathepsin B inhibitor to a subject having cancer.

In relevant aspects of this embodiment, the inhibitors of Cathepsin B include a protein, a peptide, antisense RNA, an siRNA, and a small molecule inhibitor. In a particular aspect, the Cathepsin B inhibitor is Cystatin A or a functional fragment of Cystatin A (e.g. Cystatin A₁₋₅₇), Cystatin B or a functional fragment of Cystatin B, Ca-074, Ca-074-Me, or E-64D.

In another aspect of this embodiment, the invention is directed to methods of treating cancer in a subject comprising administering a population of CAR T cells genetically modified to express or overexpress effectors of ubiquitylation to a subject having cancer.

In relevant aspects of this embodiment, the effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

In a third embodiment, the invention is directed to methods of treating cancer using CAR T cells where trogocytosis associated with CAR T cell therapy is inhibited and surface expression of cancer antigens is reduced.

Non-limiting examples of means for achieving inhibition of trogocytosis include the following. First, populations of CAR T cells can be treated with an inhibitor of trogocytosis in vitro prior to administration to a subject having cancer. Second, an inhibitor of trogocytosis can be administered to a subject being treating by CAR T cell therapy. Third, populations of CAR T cells can be genetically modified to express or overexpress an inhibitor of trogocytosis prior to administration of the cells to a subject.

Non-limiting examples of means for achieving a reduction in surface expression of cancer antigens include the following. Populations of CAR T cells can be genetically modified to express or overexpress effectors of ubiquitylation to a subject having cancer.

In one aspect of this embodiment, the invention is directed to methods of treating cancer in a subject comprising administering a population of CAR T cells genetically modified to (i) express or overexpress a Cathepsin B inhibitor and (ii) express or overexpress effectors of ubiquitylation to a subject having cancer.

In aspects of this embodiment, the inhibitors of Cathepsin B include, but are not limited to, one or more of proteins, peptides, antisense RNA, siRNA, and small molecule inhibitors. Relevant examples include, but are not limited to, Cystatin A and functional fragments thereof (e.g. Cystatin A₁₋₅₇), Cystatin B and functional fragments thereof, Ca-074, Ca-074-Me, and E-64D.

In aspects of this embodiment, the effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

In a fourth embodiment, the invention is generally directed to methods for generating trogocytosis-resistant CAR T cells. CAR T cell products can be produced and existing CAR T cell populations that exhibit a reduction in efficacy can be revived or renewed by subjecting CAR T cells to treatment with an inhibitor of trogocytosis (e.g. an inhibitor of Cathepsin B) or genetic modification to express or overexpress an inhibitor of trogocytosis (e.g. an inhibitor of Cathepsin B) as defined above.

CAR T cells can also be genetically modified to express effectors of ubiquitylation. The effects of trogocytosis can thus be reduced by targeting cancer antigens for ubiquitylation after they have been taken up by CAR T cells. By inhibiting the cell surface display of cancer antigens, the incidence of fratricide can be reduced or even eliminated in CAR T cells.

In combination, the CAR T cells can be (i) treated with an inhibitor of trogocytosis (e.g. an inhibitor of Cathepsin B) or genetically modified to express or overexpress an inhibitor of trogocytosis (e.g. an inhibitor of Cathepsin B) as defined above and (ii) genetically modified to express effectors of ubiquitylation.

The invention thus includes methods of inhibiting trogocytosis in a population of CAR T cells comprising contacting the CAR T cells with a Cathepsin B inhibitor.

The invention also includes methods for decreasing trogocytosis in a population of CAR T cells comprising genetically modifying the population of CAR T cells to express or overexpress a Cathepsin B inhibitor.

The invention further includes methods for generating trogocytosis-resistant CAR T cells comprising genetically modifying the population of CAR T cells to express or overexpress a Cathepsin B inhibitor.

The invention yet further includes methods for decreasing surface expression of cancer antigens resulting from trogocytosis in a population of CAR T cells comprising genetically modifying the population of CAR T cells to express or overexpress effectors of ubiquitylation.

The invention still further includes methods for targeted degradation of antigen transferred to CAR T cells via trogocytosis comprising genetically modifying the population of CAR T cells to express or overexpress effectors of ubiquitylation.

In a fifth embodiment, the invention is generally directed to methods of assaying for inhibitors of trogocytosis. As described in more detail below, a Luciferase-Complementation (CompLuc) Assay has been developed for the real-time detection of trogocytosis. In this assay, CAR T cells can be engineered to express an N-terminal fragment of NanoLuc luciferase (nLuc) while tumor cells can be engineered to express a cancer antigen (e.g. CD19) fused to a C-terminal NanoLuc fragment (cLuc) having affinity for nLuc. Presence of both fragments in the same cell, following cancer antigen transfer to the CAR T cell via trogocytosis, results in functional reconstitution of NanoLuc and luminescence, which can be quantified in real-time. The CompLuc Assay can be used to assay selected inhibitors for trogocytotic inhibitory activity.

In a non-limiting example, the method comprises (a) genetically modifying CAR T cells to express a N-terminal fragment of NanoLuc luciferase (nLuc), (b) genetically modifying tumor cells to express a cancer antigen fused to a C-terminal NanoLuc fragment (cLuc), (c) culturing the cells of (a) and (b) in the presence of a selected inhibitor under conditions promoting trogocytosis, and (d) assaying the cells of (a) for luminescence. When luminescence is detected in the CAR T cells modified to express nLuc, it is determined that the selected inhibitor does not inhibit trogocytosis. When luminescence is not detected in the CAR T cells modified to express nLuc, it is determined that the selected inhibitor inhibits trogocytosis.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Targeted tumor antigen is extracted from the tumor cell surface (pink cell) and incorporated into the CAR T cell surface (purple cell). This leads to the emergence of antigen-negative tumor cells which exhibit immune escape (right panel) and the emergence of antigen-positive CAR T cells, which are killed by other CAR T cells via “fratricide” (left panel).

FIG. 2. Confirmation of the implications of CAR T cell-mediated trogocytosis. A. shows the levels of CD19 levels on normal T cells and CAR T cells as determined by flow cytometry, with the panel on the left showing a control where the cells were stained with anti-hCD3, anti-hCD4, anti-hCD8, anti-CAR, anti-hCD27, anti-hCD137, and 7-AAD viability dye, and the panel on the right showing cells stained with all antibodies used in the left panel and anti-hCD19 antibodies. B. shows live T cells counts from in experiments having target-effector ratios of 0:1, 0.5:1 and 2:1, where target cells were CAR T cells (FMC63) or ΔscFv CAR T cells.

FIG. 3. Data from a novel kinetic assay for the real-time assessment of trogocytosis. A. Schema of CompLuc for the real-time detection of CAR-mediated trogocytosis. Tumor cell is in pink; CAR T cell is in purple; tumor protein CD19 is red; CAR is blue; NanoLuc fragments are white (left) or green (right). B. Schema of constructs used for production of nLuc expressing CAR T cells, CD19-cLuc expressing tumor cells, and tr19-cLuc-expressing cells. C. Determination of luciferase complementation following transient transfection, as determined by plate-based luminescence. 293T cells were transiently transfected with nLuc, cLuc, or both nLuc and cLuc. D. Determination of luciferase complementation following gamma retrovirus transduction, as determined by plate-based luminescence. E. Killing of CD19-positive Burkitt's lymphoma cell line Raji expressing firefly luciferase (Raji-luc) by CD19 CAR T cells (FMC63) or T cells expressing a CAR without a binding domain (ΔscFv) as determined by plate based luminescence. F. Assessment of the overall sensitivity of CompLuc. T cells expressing nLuc and a tr19-cLuc fusion protein were serially diluted and incubated with furimazine (the recommended NanoLuc substrate) for 5 minutes. Luminescence was measured following the incubation. G. Kinetic assessment of CAR-mediated trogocytosis using CompLuc. CAR T cells at the indicated effector-target ratios were incubated with K562 tumor cells expressing tr19-cLuc (left, orange) or CD19-cLuc (right, blue). H. Area under curve analysis for total luminescence in G. Values were calculated using “area under curve” analysis in GraphPad Prism 10. **** indicates that p<0.0001. I. Kinetic assessment of CAR-mediated trogocytosis using CompLuc. nLuc expressing CAR T cells (FMC63 [blue] or ΔscFv [grey]) were cocultured with CD19-cLuc-expressing K562 cells for 3 hours at the following effector-target ratios: 2:1, 1:1, 0.5:1, 0.25:1, 0.125:1, 0:1. J. Area under curve analysis for total luminescence in I. Values were calculated using “area under curve” analysis in GraphPad Prism 10. **** indicates that p<0.0001. K. Determination of CD19 transfer to CAR T cells as measured by flow cytometry. ΔscFv or FMC63 CAR T cells were cocultured with CD19-cLuc-expressing K562 tumor cells at a 0.25:1 effector-target ratio for one hour prior to analysis.

FIG. 4. CompLuc validates established modulators of trogocytosis and implicates Cathepsin B as key modulator. A. Amount of trogocytosis during 3-hour coculture of FMC63 CAR T cells pre-treated with the indicated concentrations of the actin polymerization inhibitor Cytochalasin D and CD19-cLuc-expressing K562 tumor cells at a 1:1 effector-target ratio, as determined by CompLuc assay. CAR T cells treated with DMSO were used as a control. Data are represented as area under curve, calculated in GraphPad Prism 10. B. Killing of CD19-positive Raji-luc cells by FMC63 CAR T cells treated with 80 μM of Cytochalasin D. CAR T cells were cocultured with Raji-luc cells at the indicated effector-target ratios and incubated for 16 hours at 37 C/5% CO₂. C. Amount of trogocytosis during 3-hour coculture of FMC63 CAR T cells pre-treated with the indicated concentrations of the following inhibitors: Dynasore (Dynamin), Ca-074-Me (Cathepsin B), PitStop (Clathrin), or BI-1950 (LFA-1), with CD19-cLuc-expressing K562 cells at a 1:4 effector-target ratio. CAR T cells treated with DMSO were used as a control. Data are represented as area under curve, calculated in GraphPad Prism 10. D. Killing of CD19-positive Raji cells expressing firefly luciferase by CD19 CAR T cells pre-treated for one hour with one of the following inhibitors: Dynasore (50 μM, Dynamin), Ca-074-Me (10 μM, Cathepsin B), PitStop (25 μM, Clathrin), BI-1950 (10 μM, LFA-1). CAR T cells were cocultured with Raji cells at the indicated effector-target ratios and incubated for 16 hours at 37 C/5% CO₂. E. CD19 transfer to CAR T cells as measured by flow cytometry. CAR T cells were pre-treated with 10 μM Ca-074-Me and cocultured with CD19-cLuc-expressing K562 tumor cells at a 1:4 effector-target ratio for one hour. Cells were stained with anti-CD19/FITC for the detection of CD19.

FIG. 5. Overexpression of the endogenous Cathepsin B inhibitors Cystatin A and Cystatin B lead to a reduction in trogocytosis. A. Crystal structures of bovine Cathepsin B in complex with Ca-074 (left, PDB: 1QDQ) and human Cathepsin B in complex with Cystatin A (right, PDB: 3K9M). The active site cysteine of Cathepsin B is shown in pink; Ca-074 and Cystatin A are shown in blue. B. Schema of construct used for the production of Cystatin A-overexpressing, nLuc expressing CAR T cells. C. Cystatin overexpression in lysates from ΔscFv and FMC63 CAR T cells was determined using a commercial Cystatin A ELISA kit. D. Expansion of ΔscFv and FMC63 CAR T cells overexpressing Cystatin A during manufacturing, as determined by automated cell counting. E. Killing of Raji-luc cells by FMC63 or ΔscFv CAR T cells with or without overexpression of Cystatin A, as determined by luminescence. F. Kinetic assessment of CAR-mediated trogocytosis using CompLuc assay. K562 tumor cells expressing CD19-cLuc were incubated with nLuc expressing CD19 CAR T cells or nLuc expressing CD19 CAR T cells overexpressing Cystatin A. G. Area under curve analysis for total luminescence in F. H. Transfer of CD19 to CAR T cells was determined via flow cytometry. I. CD19 loss on tumor cells was determined via flow cytometry. J. Percentage of CAR T cells negative for CD19 were quantified via flow cytometry. K. Total CAR T cells were quantified via flow cytometry and normalized to ΔscFv. L. CD19 transfer to CAR T cells was determined via flow cytometry. M. CD19 loss on K562 tumor cells was determined via flow cytometry.

FIG. 6. Overexpression of truncated form of Cystatin A (CSTA₁₋₅₇) leads to a similar reduction in trogocytosis. A. Schema of construct used for the production of Cystatin A-overexpressing, nLuc expressing CAR T cells. B. Expansion of ΔscFv and FMC63 CAR T cells overexpressing Cystatin A during manufacturing, as determined by automated cell counting. C. Killing of Raji-luc cells by FMC63 or ΔscFv CAR T cells with or without overexpression of Cystatin A, as determined by luminescence. CAR T cells were cocultured with Raji cells at the indicated effector-target ratios and incubated for 16 hours at 37 C/5% CO₂. D. Kinetic assessment of CAR-mediated trogocytosis using CompLuc assay. K562 tumor cells expressing CD19-cLuc were incubated with nLuc expressing CD19 CAR T cells or nLuc expressing CD19 CAR T cells overexpressing Cystatin A(₁₋₅₇)- or Cystatin B. Optimized Live Cell Substrate (Promega N2011) was used in place of the substrate furimazine. Luminescence was measured every minute for 3 hours. E. Area under curve analysis for total luminescence in D. F-H: CD19-cLuc-expressing K562 cells were cocultured with CAR T cells or CAR T cells overexpressing Cystatin A at a 1:1 effector-target ratio for 30 minutes. F. Transfer of CD19 to CAR T cells was determined via flow cytometry. G. CD19 loss on tumor cells was determined via flow cytometry. H. Total CAR T cells were quantified via flow cytometry.

FIG. 7. Development of a novel system for the degradation of trogocytosed antigen. A. Schema of a system for the degradation of trogocytosed antigen. A CD19-GFP fusion protein is transferred from the tumor cell (pink) to the CAR T cell (purple) via trogocytosis. Following trogocytosis, a GFP-targeting nanobody fused to Nslmb (TAD) binds to the CD19-GFP fusion protein, which is then polyubiquitylated. Polyubiquitylation targets the CD19-GFP fusion protein for proteasome-mediated degradation. B. Schema of constructs used to assess CD19 degradation. Top: CAR T cell construct for the co-expression of nLuc and TAD; bottom: tumor cell construct for the expression of a CD19-GFP fusion protein. C. Proof-of-concept to demonstrate the effect of TAD expression on CD19-GFP. CD19-GFP-expressing 293T cells were transiently transfected with a plasmid encoding TAD. 24 hours after transfection, GFP fluorescence was measured to assess CD19-GFP expression. D-E. CD19-cLuc-expressing K562 cells were cocultured with CAR T cells±Cystatin overexpression±TAD at a 1:1 effector-target ratio for 30 minutes. D. CD19 expression on CAR T cells was determined via flow cytometry. E. Total CAR T cell counts were assessed via flow cytometry and normalized to ΔscFv. F. Expression of CAR with or without TAD after staining with an anti-HA antibody recognizing an HA tag within the CAR, as measured via flow cytometry on Day 7 of manufacturing. G. Expansion of CAR T cells with or without TAD during manufacturing, as measured by automated cell counting. H. Killing of Raji-luc cells by CAR T cells with or without the expression of Nslmb-VHH. CAR T cells and tumor cells were cocultured at the indicated effector-target ratios for 16 hours. Tumor survival was assessed by luminescence. I. Schema of constructs used to assess a fully-human CD19 degradation system. Top: CAR T cell construct for the co-expression of CAR, nLuc, and lyn(SH2)-VHL fusion protein; middle: construct for the co-expression of a Lyn(kinase)-VHL fusion protein and GFP; bottom: construct for the co-expression of a Lyn(kinase)-CRBN fusion protein and GFP. J. Left: CD19 degradation by TAD as measured by western blot using lysates from 293T cells expressing CD19 and transiently transfected with TAD or fully human degradation constructs. Cells were lysed 24 hours after transfection. Right: quantification of bands normalized to beta-actin control. K. Left: CD19 degradation by TAD as measured by western blot using lysates from 293T cells expressing CD19-GFP and transiently transfected with TAD or a fully human degradation construct using the Lyn(SH2) domain. Cells were lysed 24 hours after transfection. Right: quantification of bands normalized to beta-actin control. L. Left: expression of Lyn(SH2)-VHL fusion protein as measured by western blot following production of CD19 CAR T cells transduced with the indicated constructs. Cells were lysed following CAR T cell production. Right: quantification of bands normalized to beta-actin control.

All data shown represents mean of three values ±s.d. All statistical significance was determined using unpaired t-test. Significance was defined as p<0.05. * indicates p<0.05; ** indicates p<0.01; *** indicates p<0.001; **** indicates that p<0.0001.

FIG. 8. Sequence Listing.

FIG. 9. CD19 and BCMA CAR T cells rapidly acquire target antigen via trogocytosis. A. Schema of CAR-mediated trogocytosis (CMT). B. CD19 transfer to CAR T cells lacking an antigen binding domain (ΔscFv) or CD19 CAR T cells (FMC63) after a 1-hour coculture with CD19-positive cell lines at a 0.5:1 effector-target ratio using flow cytometry. C. CD19 loss on tumor cells after a 1-hour coculture with ΔscFv or FMC63 CAR T cells at a 0.5:1 effector-target ratio using flow cytometry. D. Observation of CMT in brightfield and confocal images of ΔscFv or FMC63 CAR T cells cultured with CD19-GFP-expressing 293T cells. CD19 is shown in green. Scale bar represents 5p m. E. Schema of different BCMA-targeting CAR T cells. F. BCMA transfer to CAR T cells and BCMA loss on BCMA-GFP-expressing K562 cells after coculture with CAR_{BCMA} T cells. G. BCMA loss on MM.1S, RPMI8226, and U266B1 cells when cocultured with CAR_{BCMA}, Abecma, and Carvykti CAR T cells. H.-I. Amount of CD19 on CAR T cells isolated from patients 7-28 days after receiving CD19 CAR T cell therapy using full-spectrum flow cytometry. Data indicate individual patients. Statistical significance was determined by Wilcoxon matched-pairs signed-rank test.

FIG. 10. CAR-mediated trogocytosis directly causes CAR T cell dysfunction. A. Schema of trogocytosed antigen degrader (TAD). B. CD19 and C. GFP expression on FMC63 CAR T cells expressing TAD_{GFP} or T cells expressing a CAR lacking a binding domain (ΔscFv) following a 1-hour coculture with CD19-GFP-expressing NALM6 cells as determined by flow cytometry. D. Quantification of CAR T cells after a 24-hour coculture with CD19-GFP-expressing NALM6 cells using flow cytometry. Values are normalized to wells containing only CAR T cells. E. Schema of a fully human-derived TAD system (TAD_{CD19}). VHL=von Hippel-Lindau protein; Ub=ubiquitin. F. GFP and G. CD19 expression on FMC63 CAR T cells expressing TAD_{CD19} or ΔscFv CAR T cells after a 1-hour coculture with CD19-GFP-expressing NALM6 cells at a 0.5:1 effector-target ratio. H. Expression of LAG-3 and I. TIM-3 on CAR T cells expressing TAD_{GFP} after a 24-hour coculture with CD19-GFP-expressing NALM6 cells using full-spectrum flow cytometry. (B-I) Statistical significance was determined by two-tailed Student's t-test. J. Schema of in vitro serial coculture experiment to determine long-term CAR T cell expansion and antitumor activity. K. Survival of CD19-GFP-expressing NALM6 cells expressing firefly luciferase (Fluc) at a 0.5:1 effector-target ratio using a luminescence-based cytotoxicity assay. Data indicates mean±S.D. from three technical replicates. Tumor survival was normalized to untreated tumor cells. Statistical significance was determined by multiple t-tests and corrected for multiple comparisons using the Bonferroni-Dunn method.

FIG. 11. CAR-mediated trogocytosis directly causes CAR T cell dysfunction. A. Expression of CD19 on 293T cells with or without TAD constructs as determined by western blot. B. Quantification of western blot in (A). Values are normalized to β-actin. C. Fold change expansion of FMC63 CAR T cells±TAD_{GFP}±TAD_{CD19} during manufacturing as determined by cell counting. D. Survival of CD19-GFP-expressing NALM6-Fluc cells after 16-hour coculture with FMC63 CAR T cells±TAD_{GFP}±TAD_{CD19} or ΔscFv CAR T cells at the indicated effector-target ratios. Tumor survival was measured using a luciferase-based cytotoxicity assay. Data represent mean±S.D. of three technical replicates. E. Mean fluorescence intensity of Ki67 in CAR T cells after a 24-hour coculture with CD19-GFP-expressing NALM6 cells, as measured by flow cytometry. Statistical significance was determined by two-tailed Student's t-test. F. Schema of CAR and target CD19 binding in-cis following trogocytosis, resulting in persistent CAR signaling.

FIG. 12. Luciferase complementation assay allows for the real-time detection of trogocytosis. A. Schema of luciferase complementation assay (CompLuc). B. Schema of constructs used for CAR T cells (I) and tumor cells (II/III) for development of luciferase complementation assay. SP=signal peptide; HA=hemagglutinin tag; TM=transmembrane region; ECD=extracellular domain; trECD=truncated extracellular domain; ICD=intracellular domain. C. Luminescence of nLuc+cLuc+ CAR T cells. Data represent mean±S.D. of three technical replicates. D. CMT during coculture of FMC63 CAR T cells expressing nLuc with K562 cells expressing full length (teal) or truncated (tr19, orange) CD19 fused to cLuc using CompLuc or flow cytometry after 1 hour at the indicated effector-target ratios. CMT using CompLuc was measured for a total 2.5 hours at 1-minute intervals. Data represents best fit of three technical replicates. E. Area-under-curve quantification of luminescence in FIG. 10D. F. CMT during coculture of ΔscFv or FMC63 CAR T cells expressing nLuc with K562 cells expressing full length CD19-cLuc using CompLuc or flow cytometry after 1 hour at the indicated effector-target ratios. CMT using CompLuc was measured for a total 2.5 hours at 1-minute intervals. Data represents best fit of three technical replicates. G. Peak luminescence of FMC63 CAR T cells cocultured with K562 cells expressing CD19-cLuc at a 1:1 effector-target ratio as determined by CompLuc. CAR T cells were pre-treated with cytochalasin D for 1 hour at the indicated concentrations. H. Survival of Raji-Fluc cells after 16-hour coculture with FMC63 CAR T cells pre-treated with the indicated concentrations of cytochalasin D. CMT values are normalized to a DMSO control. (G/H) Data represent mean±S.D. of three technical replicates.

FIG. 13. Extracellular cathepsin B is a key driver of CAR-mediated trogocytosis. A. Schema of potential modulators of CMT. B. Peak luminescence as determined by CompLuc of FMC63 CAR T cells treated with inhibitors of the indicated proteins cocultured with K562 cells expressing CD19-cLuc at a 1:1 effector-target ratio. Values are normalized to a DMSO control. C. Survival of Raji-Fluc cells after 16-hour coculture with FMC63 CAR T cells treated with inhibitors of the indicated proteins at a single concentration. Tumor survival was measured using a luciferase-based cytotoxicity assay. D. Peak luminescence as determined by CompLuc of FMC63 CAR T cells cocultured with K562 cells expressing CD19-cLuc at a 1:1 effector-target ratio. FMC63 CAR T cells were pre-treated with the indicated concentrations of Ca-074-Me (membrane permeable) or Ca-074 (membrane impermeable). Values are normalized to a DMSO control. (B-D) Data represent mean±S.D. of three technical replicates. E. Schema of microscopy setup used to assess cathepsin B (CTSB) localization to the immune synapse. Biotinylated CD19 was immobilized on plate-bound NeutrAvidin. FMC63 CAR T cells expressing CTSB-mCherry were added to slides and imaged using confocal microscopy. F. Axial dispersion of CTSB in fixed ΔscFv and FMC63 CAR T cells 5 minutes after plating. G. Average distance of CTSB to immune synapse in fixed ΔscFv and FMC63 CAR T cells 5 minutes and 15 minutes after plating. (F/G) Statistical significance was determined by two-tailed Student's t-test. H. Side (x-z) view of the distribution of CTSB-mCherry in ΔscFv or FMC63 CAR T cells on immobilized CD19 at indicated time points using spinning disk confocal imaging.

FIG. 14. Cystatin abundance controls CTSB activity and CMT. A. Crystal structures of bovine cathepsin B in complex with Ca-074 (left, PDB: 1QDQ) and human cathepsin B in complex with cystatin A (right, PDB: 3K9M). The active site cysteine of cathepsin B is shown in pink; Ca-074 and cystatin A are shown in teal. B. Levels of CSTA in CAR or CAR_{CSTA} T cells measured by ELISA. C. Cathepsin B activity in CAR or CAR_{CSTA} T cells measured by fluorescence. (J/K) Data represent mean±S.D. of two technical replicates. Statistical significance was determined by two-tailed Student's t-test. D. Survival of Raji-Fluc cells after a 16-hour coculture with FMC63 CAR or CAR_{CSTA} T cells at the indicated effector-target ratios. Data represent mean±S.D. of three technical replicates. E. CMT of FMC63 CAR or CAR_{CSTA} T cells as determined by CompLuc using K562 cells expressing CD19-cLuc after 3 hours. F. CMT of BCMA CAR or CAR_{CSTA} T cells as determined by CompLuc using K562 cells expressing BCMA-GFP-cLuc after 3 hours. (E/F) Data represents best fit of three technical replicates. G. CD19 transfer to CAR T cells following a 30-minute coculture of FMC63 CAR or CAR_{CSTA} T cells and K562 cells expressing CD19-cLuc at a 0.5:1 effector-target ratio. H. BCMA-GFP transfer to CAR T cells following a 2-hour coculture of BCMA CAR or CAR_{CSTA} T cells and K562 cells expressing BCMA-GFP-cLuc at a 1:1 effector-target ratio. I. CD19 loss on tumor cells following a 30-minute coculture of FMC63 CAR or CAR_{CSTA} T cells and K562 cells expressing CD19-cLuc at a 0.5:1 effector-target ratio. J. BCMA loss on U266B1 tumor cells following a 2-hour coculture of BCMA CAR or CAR_{CSTA} T cells and K562 cells expressing BCMA-GFP-cLuc at a 1:1 effector-target ratio. K. Total CAR T cells following a 30-minute coculture of FMC63 CAR or CAR_{CSTA} T cells and K562 cells expressing CD19-cLuc at a 0.5:1 effector-target ratio. L. total CAR T cells following a 2-hour coculture of BCMA CAR or CAR_{CSTA} T cells and K562 cells expressing BCMA-GFP-cLuc at a 1:1 effector-target ratio. (K/L) CAR T cell numbers are normalized to wells containing only CAR T cells using counting beads. (G-L) Statistical significance was determined by two-tailed Student's t-test.

FIG. 15. Cystatin A overexpression enhances long-term CAR T-cell persistence. a. Schema of the in vitro serial cytotoxicity experiment to determine long-term CAR T-cell persistence and antitumor activity. b. Survival of CD19-GFP-expressing NALM6-Fluc cells at a 0.5:1 effector-target ratio using a luminescence-based cytotoxicity assay. Data indicate the mean±S.D. from three technical replicates. Tumor survival was normalized to untreated tumor cells. Data are representative of two independent experiments. Statistical significance comparing FMC63 CAR and CAR_{CSTA} T cells was determined by multiple two-tailed Student's t tests. c. FMC63 CAR and CAR_{CSTA} T-cell counts on days 1-3 of the in vitro serial coculture described in FIG. 15a. Data represent the mean±S.D. of 3 technical replicates and are representative of two independent experiments. Statistical significance comparing FMC63 CAR and CAR_{CSTA} T cells was determined by multiple two-tailed Student's t tests. d. Schema of the in vivo experiment to measure NALM6 tumor control and CAR T-cell persistence. e. Tumor burden in mice bearing systemic NALM6-Fluc tumors and treated with ΔscFv or FMC63 CAR or CAR_{CSTA} T cells as determined by an in vivo imaging system (IVIS). Data represent the mean±S.D. of 5 mice per group. f. Quantification of CAR T cells in murine spleen, peripheral blood, and bone marrow as determined by flow cytometry. Data represent the mean±S.D. of 10 mice pooled from two independent experiments, with experiments denoted by symbols. Data were normalized to the average of the respective experiment's CSTA⁻ condition and are expressed as fold change. Statistical significance was determined by two-tailed Student's t test. g. Phenotype of FMC63 CAR/CAR_{CSTA} T cells from murine blood as determined by flow cytometry. Statistical significance was determined by two-way ANOVA. h. PD-1, TIM-3, and LAG-3 expression on FMC63 CAR or CAR_{CSTA} T cells isolated from murine blood as determined by flow cytometry. Statistical significance was determined by two-tailed Student's t test. g/h. Data represent the mean±S.D. of 5 mice per group. i. Differential mRNA expression in sorted FMC63 CAR and CAR_{CSTA} T cells from two independent productions at the end of manufacturing as determined by bulk RNA sequencing. j. Schema of the in vitro serial cytotoxicity experiment to determine long-term CAR T-cell persistence and antitumor activity. k. Survival of NALM6-Fluc cells at a 2:1 effector target ratio using a luminescence-based serial cytotoxicity assay. On Days 2, 4, 6, 8, 10, 12, and 14, CAR T cells were normalized, and fresh tumor cells were added. 1. Total CAR T cells on day 10 of the in vitro serial coculture described in FIG. 15j. m. PD-1 expression on CAR T cells on day 5 of in vitro serial coculture described in FIG. 15j. n. TIM-3 expression on CAR T cells on day 5 of in vitro serial coculture described in FIG. 15j. k-n. Data represent the mean±S.D. of three technical replicates and are representative of two independent experiments. o. Quantification of CAR T cells in bone marrow 28 days after intravenous treatment with the indicated CAR T-cell conditions as determined by flow cytometry. Data represent the mean±S.D. of 2-5 mice per group. Statistical significance was determined by one-way ANOVA. p. Schema of the in vivo experiment to measure solid tumor control and CAR T-cell infiltration into solid tumors. q. Tumor burden in mice bearing intratibial CD19-expressing A673-Fluc tumors and treated intravenously with ΔscFv or FMC63 CAR CISH^{WT} or CAR_{CSTA} CISH^{KO} T cells as determined by an in vivo imaging system (IVIS). Data represent the mean±S.D. of 3-4 mice per group. r. Quantification of CAR T cells per 1,000 acquired live cells in peripheral blood four days after treatment with the indicated CAR T-cell conditions as determined by flow cytometry (n=2-3 per group). Statistical significance was determined by one-way ANOVA. s. Quantification of CAR T cells per 1,000 acquired live cells in intratibial A673 tumors four days after treatment with the indicated CAR T-cell conditions as determined by flow cytometry (n=2-3 per group). Statistical significance was determined by one-way ANOVA. t. PD-1 and u. TIM-3 levels on intratumoral CAR T cells four days after CAR T-cell injection as determined by flow cytometry. Statistical significance was determined by one-way ANOVA. r-u. Data represent the mean±S.D. of 2-3 mice per group.

FIG. 16. CAR_{CSTA} T cells retain benefits of reduced trogocytosis following CISH knockout. a. Tumor burden in 5 mice per group bearing systemic NALM6 tumors and treated with intravenous ΔscFv, FMC63 CAR, or FMC63 CAR_{CSTA} T cells as determined by IVIS. Data is representative of two independent experiments. b. Amounts of TIM-3 in FMC63 CAR or CAR_{CSTA} T cells throughout an in vitro serial coculture with CD19-GFP-expressing NALM6-luc cells as determined by flow cytometry. Statistical significance was determined using multiple two-tailed Student's t-tests. Data represent mean±S.D. of 3 technical replicates. Data is representative of two independent experiments. c. Western blot showing expression of CISH in CAR_{CSTA} CISH^{WT} and CISH^{KO} T cells at the end of manufacturing. d. Quantification of western blot in FIG. 16c. Values are normalized to β-actin. e. Phenotype of FMC63 CAR_{CSTA} CISH^{WT} or CISH^{KO} CAR T cells following production as determined by flow cytometry. f. Amount of CD19 on CAR T cells following a 1-hour coculture of FMC63 CAR, CAR_{CSTA} CISH^{WT}, or CAR_{CSTA} CISH^{KO} T cells and NALM6 cells expressing CD19-GFP at a 1:1 effector-target ratio as determined by flow cytometry. g. Amount of CD19 on tumor cells following a 1-hour coculture of FMC63 CAR, CAR_{CSTA} CISH^{WT}, or CAR_{CSTA} CISH^{KO} T cells and NALM6 cells expressing CD19-GFP at a 1:1 effector-target ratio as determined by flow cytometry. h. Number of CART cells following a 1-hour coculture of FMC63 CAR, CAR_{CSTA} CISH^{WT}, or CAR_{CSTA} CISH^{KO} T cells and NALM6 cells expressing CD19-GFP at a 1:1 effector-target ratio as determined by flow cytometry. CAR T cell numbers are normalized to wells containing only CAR T cells using counting beads. f-h. Statistical significance was determined using one-way ANOVA. Data represent mean±S.D. of 3 technical replicates. Data is representative of three independent experiments. i. Tumor burden in 5 mice per group bearing systemic NALM6 tumors and treated with intravenous ΔscFv, FMC63 CAR, or FMC63 CAR_{CSTA} CISH^{WT} or CISH^{KO} T cells as determined by IVIS. j. Tumor burden in 3-4 mice per group bearing intratibial A673 tumors and treated with intravenous ΔscFv, FMC63 CAR, or FMC63 CAR_{CSTA} CISH^{KO} T cells as determined by IVIS. k. Phenotype of intratumoral CAR T cells from mice bearing intratibial A673 tumor cells four days after CAR T cell injection as determined by flow cytometry. Data represent mean±S.D. from 2-3 animals per group. Statistical significance was determined by two-way ANOVA.

### DETAILED DESCRIPTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

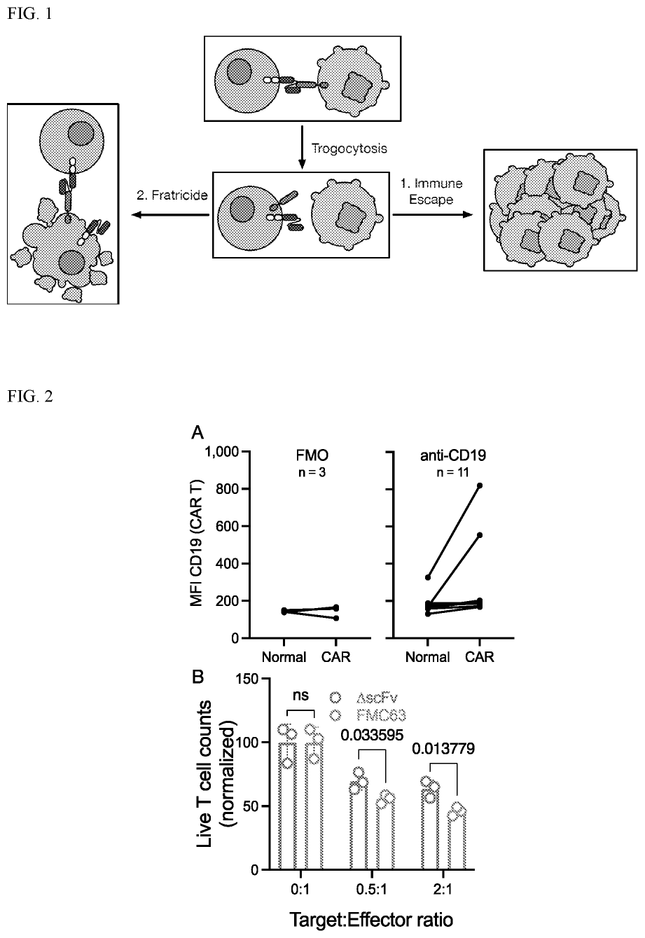

As discussed above, chimeric antigen receptor (CAR) T cell therapy is a highly effective treatment for cancer in which patient T cells are genetically modified to express a CAR targeting an antigen expressed on the surface of cancer cells [2]. CAR T cells are successful in treating various cancers but resistance to the therapy remains common [3,4]. A recently identified mechanism of treatment resistance is CAR-mediated trogocytosis (CMT). CMT is a process in which the targeted cell surface antigens are transferred from tumor cells to CAR T cells [5-7](FIG. 1). This antigen transfer leads to rapid loss of the targeted antigen on the cancer cells, rendering them resistant to CAR T cell therapy (immune escape). CMT also leads to killing of antigen-positive CAR T cells by other CAR T cells (“fratricide”) [5,6], resulting in reduced CAR T cell persistence. Both processes substantially reduce CAR T cell efficacy, but the molecular mechanisms of CMT remain poorly understood.

To aid in the understanding of CMT, and as reported herein, a Luciferase-Complementation (CompLuc) Assay has been developed for the real-time detection of CMT. In this assay, CAR T cells constitutively express an N-terminal fragment of NanoLuc luciferase (nLuc) while tumor cells express a cancer antigen (e.g. CD19) fused to a C-terminal NanoLuc fragment (cLuc) having high affinity for nLuc. Presence of both fragments in the same cell, following cancer antigen transfer to the CAR T cell via trogocytosis, results in functional reconstitution of NanoLuc and luminescence, which can be quantified in real-time.

Thus, the CompLuc assay is a novel tool for the kinetic quantification of CAR-mediated trogocytosis that can be used to determine the molecular basis of trogocytosis in general. It also has practical applications in that it can be used, for example, to identify superior T cells by identifying those constructs conferring lower amounts of trogocytosis. The assay can also be used to identify key modulators of trogocytosis.

Indeed, and as detailed in the experiments described below, the CompLuc assay was used to demonstrate inhibitors of the cysteine protease Cathepsin B could modulate trogocytosis in CAR T cells. This led to the development of a novel CAR T cell product which stably overexpresses Cystatin A, an endogenous inhibitor of Cathepsin B. As shown in the experimental data presented herein, overexpression of Cystatin A results in a population of CAR T cells that undergo decreased trogocytosis thus allowing the cells to exhibit maintained cytotoxicity and persistence.

The present invention is generally directed to novel CAR T cell populations, as well as methods of using these cells in the treatment of cancer. The following paragraphs define exemplary aspects and embodiments of the invention.

### CAR T Cells Exhibiting Reduced Trogocytosis

A first embodiment of the invention encompasses populations of CAR T cells, e.g. T cells expressing chimeric antigen receptors (CAR), that exhibit reduced trogocytosis when in contact with a target cell, such as a cancer cell, expressing the CAR-specific antigen.

A reduction in trogocytosis results in a corresponding decrease in immune escape because the levels of cancer antigen displayed by the cellular targets of the CAR T cells are not reduced, or are reduced less than would otherwise occur with existing populations of CAR T cells. Thus, the cellular targets, i.e. the cancer cells, can continue to be recognized and bound by the improved CAR T cells of the present invention, with corresponding cancer cell destruction.

The reduction in trogocytosis also reduces or eliminates the occurrence of fratricide because the improved CAR T cells of the present invention display reduced amounts of cancer antigen on their surface. Thus, the improved CAR T cells of the invention are not targeted by other CAR T cells that recognize the cancer antigens, and therapeutic levels of CAR T cells can be maintained in the subject undergoing treatment.

As described in the experiments provided below, the inventors were able to identify the cysteine protease Cathepsin B as a necessary component for trogocytosis. By inhibiting the activity of the protease, trogocytosis can be reduced or even eliminated in CAR T cells. Such inhibition can be achieved by culturing the cells in the presence of a Cathepsin B inhibitor. Alternatively, inhibition can be achieved by genetically modifying the cells to express exogenous inhibitors and/or overexpression endogenous inhibitors.

Suitable inhibitors include, but are not limited to, proteins, peptides, antisense RNA, siRNA, and small molecule inhibitors. Relevant examples include, but are not limited to, Cystatin A and functional fragments thereof, Cystatin B and functional fragments thereof, Ca-074, Ca-074-Me, and E-64D. Exemplary functional fragments of Cystatin A include, but are not limited to, Cystatin A₁₋₅₇.

The present invention thus includes populations of CAR T cells that exhibit reduced trogocytosis due to inhibition of Cathepsin B. As used throughout this disclosure, “exhibit reduced trogocytosis” means a statistically significant reduction in the amount of trogocytosis between two populations of cells. In some aspects, the reduction in trogocytosis in a population of CAR T cells of the invention is at least 5, 10, 15, 20, 25, 30, 35, 40, 45, 50, 55, 60, 65, 70, 75, 80, 85, 90 or 95% versus a population of CAR T cells not exposed to an inhibitor of Cathepsin B.

The present invention includes populations of CAR T cells that exhibit reduced trogocytosis, wherein the CAR T cells express or overexpress an inhibitor of Cathepsin B.

The present invention includes populations of CAR T cells that exhibit reduced trogocytosis are populations of CAR T cells that overexpress Cystatin A or a functional fragment of Cystatin A. Functional fragments of Cystatin A include, but are not limited to, Cystatin A¹⁻⁵⁷.

The present invention includes populations of CAR T cells that exhibit reduced trogocytosis are populations of CAR T cells that overexpress Cystatin B or a functional fragment of Cystatin B.

As also described in the experiments provided below, the inventors were able to determine that effects of trogocytosis can be reduced by targeting cancer antigens for ubiquitylation after they have been taken up by CAR T cells. By inhibiting the cell surface display of cancer antigens, the incidence of fratricide can be reduced or even eliminated in CAR T cells. Such inhibition can be achieved by genetically modifying the CAR T cells to express effectors of ubiquitylation.

The present invention thus includes populations of CAR T cells that exhibit reduced surface expression of cancer antigens, wherein the CAR T cells express or overexpress effectors of ubiquitylation.

Suitable effectors of ubiquitylation include, but are not limited to, binding proteins linking the transferred antigen to ubiquitin ligases.

For example, an ubiquitin ligase may be linked to an antibody-derived binding domain targeting the transferred antigen (e.g. a GFP-specific monobody) or a natural ligand of the antigen (e.g. Lyn SH2 domain for CD19) for the purpose of degrading trogocytosed antigens.

Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

As used throughout this disclosure, “exhibit reduced surface expression of cancer antigens” means a statistically significant reduction in the amount of a particular cancer antigen displayed on the cell surface as between two populations of cells. In some aspects, the reduction in surface expression in a population of CAR T cells of the invention is at least 5, 10, 15, 20, 25, 30, 35, 40, 45, 50, 55, 60, 65, 70, 75, 80, 85, 90 or 95% versus a population of CAR T cells not exposed to an effector of ubiquitylation B.

As further described in the experiments provided below, the two means for augmenting the effectiveness of CAR T cell therapy may be combined. Thus, the invention is also directed to populations of CAR T cells that exhibit both reduced trogocytosis when in contact with a target cell, such as a cancer cell, and reduced surface expression of cancer antigens.

A reduction in trogocytosis may be achieved by genetically modifying the CAR T cells to express or overexpress an inhibitor of Cathepsin B, thus inhibiting transfer of cancer antigens to the CAR T cells.

A reduction in surface expression of cancer antigens may be achieved by genetically modifying the CAR T cells to express or overexpress effectors of ubiquitylation, thus promoting degradation of cancer antigens that are transferred to CAR T cells.

As defined above, the inhibitor of Cathepsin B may be, but is not limited to, a protein, a peptide, antisense RNA, an siRNA, and a small molecule inhibitor. In a particular aspect, the Cathepsin B inhibitor is Cystatin A or a functional fragment of Cystatin A (e.g. Cystatin A₁₋₅₇), Cystatin B or a functional fragment of Cystatin B, Ca-074, Ca-074-Me, or E-64D.

As defined above, effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

### Methods of Treating Cancer

A second embodiment of the invention encompasses methods of using the improved CAR T cells disclosed herein in the treatment of cancer. Due to the decrease in immune escape and fratricide exhibited by the CAR T cells of the invention, the efficacy of CAR T cell therapy can be improved. As will be apparent to the skilled artisan, the improved efficacy can avoid the development of resistance to treatment. It also means treatments that become less effective in a particular subject can be revived or renewed by subjecting those CAR T cells that exhibit a reduction in efficacy to treatment with an inhibitor of trogocytosis (e.g. an inhibitor of Cathepsin B) or genetic modification to express or overexpress an inhibitor of trogocytosis (e.g. an inhibitor of Cathepsin B) as defined above. Such methods include the following.

First, populations of CAR T cells can be treated with an inhibitor of trogocytosis in vitro prior to administration to a subject having cancer. For example, populations of CAR T cells can be cultured in the presence of inhibitors added to culture media for suitable times and then administered to a subject.

As defined above, suitable inhibitors include, but are not limited to, proteins, peptides, antisense RNA, siRNA, and small molecule inhibitors. Relevant examples include, but are not limited to, Cystatin A and functional fragments thereof, Cystatin B and functional fragments thereof, Ca-074, Ca-074-Me, and E-64D. Exemplary functional fragments of Cystatin A include, but are not limited to, Cystatin A₁₋₅₇.

The amount of inhibitor added to the culture media will vary and depend on such factors as the identity of the inhibitor, the identity of the culture media, and the culture conditions. However, suitable amounts will generally fall within the range of 0.1 to 100 μM.

The invention thus includes methods of treating cancer in a subject comprising (a) contacting a population of CAR T cells with a Cathepsin B inhibitor and (b) administering the cells of (a) to a subject having cancer.

Second, an inhibitor of trogocytosis can be administered to a subject being treated by CAR T cell therapy. The inhibitor can be administered before, during or after administration of the CAR T cells to the subject.

As defined above, suitable inhibitors include, but are not limited to, proteins, peptides, antisense RNA, siRNA, and small molecule inhibitors. Relevant examples include, but are not limited to, Cystatin A and functional fragments thereof, Cystatin B and functional fragments thereof, Ca-074, Ca-074-Me, and E-64D. Exemplary functional fragments of Cystatin A include, but are not limited to, Cystatin A₁₋₅₇. The inhibitors may be administered in a pharmaceutical formulation comprising one or more inhibitors and a pharmaceutically acceptable carrier, excipient and/or diluent.

The amount of inhibitor administered to the subject will vary and depend on such factors as the identity of the inhibitor, the identity of the cancer, and relevant characteristics of the subject being treated, such as body weight.

The invention thus includes methods of treating cancer in a subject comprising administering a Cathepsin B inhibitor and a population of CAR T cells to a subject having cancer.

The invention also includes methods of augmenting anti-cancer activity of CAR T cells in a subject comprising administering a Cathepsin B inhibitor to a subject undergoing CAR T cell therapy.

The invention further includes methods of inhibiting trogocytosis in CAR T cells in a subject comprising administering a Cathepsin B inhibitor to a subject undergoing CAR T cell therapy.

Third, populations of CAR T cells can be genetically modified to express or overexpress an inhibitor of trogocytosis prior to administration of the cells to a subject. For example, inhibition can be achieved by genetically modifying the cells to express exogenous inhibitors that the T cells do not normally express, or to overexpress endogenous inhibitors expressed by the cells but not at levels sufficient to inhibit trogocytosis.

As suggested above and shown herein, it has been discovered by the inventors that inhibition of the protease Cathepsin B modulates trogocytosis experienced by CAR T cells. Thus, the populations of CAR T cells can be genetically modified to express or overexpress an inhibitor of Cathepsin B prior to administered of the cells to a subject.

As defined above, suitable inhibitors include, but are not limited to, proteins, peptides, antisense RNA, siRNA, and small molecule inhibitors. Relevant examples include, but are not limited to, Cystatin A and functional fragments thereof, Cystatin B and functional fragments thereof, Ca-074, Ca-074-Me, and E-64D. Exemplary functional fragments of Cystatin A include, but are not limited to, Cystatin A₁₋₅₇.

The invention thus includes methods of treating cancer in a subject comprising administering a population of CAR T cells genetically modified to express or overexpress a Cathepsin B inhibitor to a subject having cancer.

Fourth, populations of CAR T cells can be genetically modified to express effectors of ubiquitylation. The effects of trogocytosis can thus be reduced by targeting cancer antigens for ubiquitylation after they have been taken up by CAR T cells. By inhibiting the cell surface display of cancer antigens, the incidence of fratricide can be reduced or even eliminated in CAR T cells.

The invention thus includes methods of treating cancer in a subject comprising administering a population of CAR T cells genetically modified to express or overexpress effectors of ubiquitylation to a subject having cancer.

In relevant aspects of this embodiment, the effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

As used throughout this disclosure, “inhibiting trogocytosis”, “reducing trogocytosis” and “decreasing trogocytosis” means a statistically significant inhibition, reduction or decrease in the amount of trogocytosis between two populations of cells. In some aspects, the inhibition, reduction or decrease is at least 5, 10, 15, 20, 25, 30, 35, 40, 45, 50, 55, 60, 65, 70, 75, 80, 85, 90 or 95% versus a population of CAR T cells not exposed to an inhibitor of Cathepsin B.

As used throughout this disclosure, “augmenting anti-cancer activity” means a statistically significant increase in the amount of anti-cancer activity between two populations of CAR T cells. In some aspects, the increase is at least 5, 10, 15, 20, 25, 30, 35, 40, 45, 50, 55, 60, 65, 70, 75, 80, 85, 90 or 95% versus a population of CAR T cells not exposed to an inhibitor of Cathepsin B.

In a related third embodiment, the invention is directed to methods of treating cancer using a combination of the approaches discussed above. This, the invention is directed to methods of treating cancer using CAR T cells where trogocytosis is inhibited and surface expression of cancer antigens is reduced.

Non-limiting examples of means for achieving inhibition of trogocytosis include the following. First, populations of CAR T cells can be treated with an inhibitor of trogocytosis in vitro prior to administration to a subject having cancer. Second, an inhibitor of trogocytosis can be administered to a subject being treating by CAR T cell therapy. Third, populations of CAR T cells can be genetically modified to express or overexpress an inhibitor of trogocytosis prior to administration of the cells to a subject.

Non-limiting examples of means for achieving a reduction in surface expression of cancer antigens include the following. Populations of CAR T cells can be genetically modified to express or overexpress effectors of ubiquitylation to a subject having cancer.

In one aspect of this embodiment, the invention is directed to methods of treating cancer in a subject comprising administering to a subject having cancer a population of CAR T cells genetically modified to both (i) express or overexpress a Cathepsin B inhibitor and (ii) express or overexpress effectors of ubiquitylation.

In aspects of this embodiment, the inhibitors of Cathepsin B include, but are not limited to, one or more of proteins, peptides, antisense RNA, siRNA, and small molecule inhibitors. Relevant examples include, but are not limited to, Cystatin A and functional fragments thereof (e.g. Cystatin A₁₋₅₇), Cystatin B and functional fragments thereof, Ca-074, Ca-074-Me, and E-64D.

In aspects of this embodiment, the effectors of ubiquitylation include, but are not limited to, binding proteins linking the transferred antigen to ubiquitin ligases. Binding proteins include, but are not limited to, monobodies, scFvs, DARPINs, or any other type of antibody-derived binding domain. Alternative binding domains include natural ligands of the transferred antigen. Ubiquitin ligases include, but are not limited to, Von-Hippel-Lindau factor, cereblon, damage binding protein 1, and NSlmb.

### Methods of Producing CAR T Cells Exhibiting Reduced Trogocytosis

In a fourth embodiment, the invention is directed to methods for generating trogocytosis-resistant CAR T cells.

As detailed above, such CAR T cells will exhibit improved efficacy and inhibit the development of resistance to CAR T cell treatment. New CAR T cell products can be produced and CAR T cells that exhibit a reduction in efficacy can be revived or renewed by subjecting CAR T cells to treatment with an inhibitor of trogocytosis (e.g. an inhibitor of Cathepsin B) or genetic modification to express or overexpress an inhibitor of trogocytosis (e.g. an inhibitor of Cathepsin B) as defined above.

CAR T cells can also be genetically modified to express effectors of ubiquitylation. The effects of trogocytosis can thus be reduced by targeting cancer antigens for ubiquitylation after they have been taken up by CAR T cells. By inhibiting the cell surface display of cancer antigens, the incidence of fratricide can be reduced or even eliminated in CAR T cells.

In combination, the CAR T cells can be (i) treated with an inhibitor of trogocytosis (e.g. an inhibitor of Cathepsin B) or genetically modified to express or overexpress an inhibitor of trogocytosis (e.g. an inhibitor of Cathepsin B) as defined above and (ii) genetically modified to express effectors of ubiquitylation as defined above.

The invention thus includes methods for decreasing trogocytosis in a population of CAR T cells comprising genetically modifying the population of CAR T cells to express or overexpress a Cathepsin B inhibitor as defined above.

The invention also includes methods for decreasing trogocytosis in a population of CAR T cells comprising genetically modifying the population of CAR T cells to express or overexpress effectors of ubiquitylation as defined above.

The invention yet further includes methods for decreasing trogocytosis in a population of CAR T cells comprising (i) genetically modifying the population of CAR T cells to express or overexpress a Cathepsin B inhibitor as defined above and (ii) genetically modifying the population of CAR T cells to express or overexpress effectors of ubiquitylation as defined above.

The invention also includes methods for generating trogocytosis-resistant CAR T cells comprising genetically modifying the population of CAR T cells to express or overexpress a Cathepsin B inhibitor as defined above.

The invention further includes methods for generating trogocytosis-resistant CAR T cells comprising genetically modifying the population of CAR T cells to express or overexpress effectors of ubiquitylation as defined above.

The invention yet further includes methods for generating trogocytosis-resistant CAR T cells comprising (i) genetically modifying the population of CAR T cells to express or overexpress a Cathepsin B inhibitor as defined above and (ii) genetically modifying the population of CAR T cells to express or overexpress effectors of ubiquitylation as defined above.

The invention yet further includes methods for decreasing surface expression of cancer antigens resulting from trogocytosis in a population of CAR T cells comprising genetically modifying the population of CAR T cells to express or overexpress effectors of ubiquitylation as defined above.

The invention still further includes methods for targeted degradation of antigen transferred to CAR T cells via trogocytosis comprising genetically modifying the population of CAR T cells to express or overexpress effectors of ubiquitylation as defined above.

In each of these embodiments, the subject is a human, a non-human primate, bird, horse, cow, goat, sheep, a companion animal, such as a dog, cat or rodent, or other mammal.

### Methods of Assaying Selected Inhibitors of Trogocytosis

As described above, the CompLuc assay is a novel tool for the kinetic quantification of CAR-mediated trogocytosis that can be used to determine the molecular basis of trogocytosis in general.

The present invention makes use of the CompLuc Assay defined above to screen for inhibitors of trogocytosis. This assay was used to identify specific inhibitors of Cathepsin B as inhibitors of trogocytosis.

In a non-limiting example, the present invention includes methods of assaying for inhibitors of trogocytosis. The method comprises (a) genetically modifying CAR T cells to express a N-terminal fragment of NanoLuc luciferase (nLuc), (b) genetically modifying tumor cells to express a cancer antigen fused to a C-terminal NanoLuc fragment (cLuc), (c) culturing the cells of (a) and (b) in the presence of a selected inhibitor under conditions promoting trogocytosis, and (d) assaying the cells of (a) for luminescence. When luminescence is detected in the CAR T cells modified to express nLuc, it is determined that the selected inhibitor does not inhibit trogocytosis. When luminescence is not detected in the CAR T cells modified to express nLuc, it is determined that the selected inhibitor inhibits trogocytosis.

The present invention also includes methods for screening populations of CAR T cells for decreased levels of trogocytosis. The method comprises (a) genetically modifying CAR T cells to express a N-terminal fragment of NanoLuc luciferase (nLuc), (b) genetically modifying tumor cells to express a cancer antigen fused to a C-terminal NanoLuc fragment (cLuc), (c) culturing the cells of (a) and (b) under conditions promoting trogocytosis, and (d) assaying the cells of (a) for luminescence. The amounts of luminescence detected can be compared with the luminescence detected in other populations of CAR T cells to identify CAR T cells that exhibit decreased levels of trogocytosis.

### Pharmaceutical Compositions

In each of the aspects and embodiments of the invention, the CAR T cells and the inhibitors of Cathepsin B may be administered to a subject in the form of a pharmaceutical composition. The pharmaceutical compositions may comprise only the CAR T cells, only the inhibitors of Cathepsin B, or both the CAR T cells and the inhibitors of Cathepsin B, along with one or more pharmaceutically acceptable carriers, excipients and/or diluents.

The pharmaceutical compositions may be formulated for and administered by, for example, oral, sublingual, intranasal, intraocular, rectal, transdermal, mucosal, pulmonary, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

Pharmaceutically acceptable carriers, excipients and diluents are those compounds, solutions, substances or materials that can be used to produce formulations of the CAR T cells and/or the inhibitors of Cathepsin B that are suitable to be administered to a subject, such as a human. In particular, carriers, excipients and diluents of the present invention are those useful in preparing a pharmaceutical composition that is generally safe, non-toxic and neither biologically nor otherwise undesirable and that may present pharmacologically favorable profiles, and includes carriers and diluents that are acceptable for veterinary use as well as human pharmaceutical use. Suitable pharmaceutically acceptable carriers, excipients and diluents are well known in art and can be determined by those of skill in the art as the clinical situation warrants. Examples of suitable carriers and diluents include dextrose, water, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. More particularly: (1) 5% (w/v) dextrose, or (2) water (e.g., sterile water; Water-For-Injection), may be used as a pharmaceutically acceptable carrier. Pharmaceutically acceptable diluents also include tonicity agents that make the composition compatible with blood. Tonicity agents are particularly desirable in injectable formulations.

Excipients included in a formulation have different purposes depending, for example on the nature of the active agent and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweeteners, perfuming agents, flavoring agents, coloring agents, administration aids, and combinations thereof.

The pharmaceutical compositions may contain common carriers and excipients, such as cornstarch or gelatin, lactose, sucrose, microcrystalline cellulose, kaolin, mannitol, dicalcium phosphate, sodium chloride, alginic acid, croscarmellose sodium, and sodium starch glycolate.

The particular carrier, diluent or excipient used will depend upon the means and purpose for which the active ingredient is being applied.

Acceptable methods for preparing the pharmaceutical compositions according to the invention are known to those skilled in the art. For example, pharmaceutical compositions may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

### Methods of Treatment

The methods of the invention include methods of treating cancer in a subject generally comprising administering therapeutically-effective amounts the CAR T cells of the present invention and/or the inhibitors of Cathepsin B to a subject having cancer. When both are used, the CAR T cells and the inhibitors of Cathepsin B may be administered in any order, separately or in combination, sequentially or concurrently, with overlapping or non-overlapping periods of administration. The methods of the invention thus include methods of treating cancer in a subject, comprising concurrently administering therapeutically effective amounts of the CAR T cells and the inhibitors of Cathepsin B to a subject having cancer. The methods of the invention thus also include methods of treating cancer in a subject, comprising sequentially administering therapeutically effective amounts of the CAR T cells and the inhibitors of Cathepsin B to a subject having cancer. The methods of the invention further include methods of treating cancer in a subject, comprising concurrently administering therapeutically effective amounts of the CAR T cells of the present invention to a subject having cancer.

The terms “treating” and “treatment” mean at least the mitigation of cancer, or a disease condition or symptom associated with cancer in a subject that is achieved by a reduction of growth, replication, and/or propagation, or death or destruction of cancer and/or cancer cells, on or in the subject. The terms “treating” and “treatment” include curing, healing, inhibiting, relieving from, improving and/or alleviating, in whole or in part, the cancer or associated disease condition or symptom. The mitigation of cancer or associated disease condition or symptom may be about 100%, 99%, 98%, 97%, 96%, 95%, 90%, 80%, 70%, 60%, 50%, 40%, 30%, 20%, 10%, 5% or 1% in the subject, versus a subject to which the CAR T cells of the present invention have not been administered. In one aspect, treating means reducing the population of cancer cells causing the cancer in the subject to an undetectable level, where detection is by any conventional means, such assay a blood sample in the laboratory. In another aspect, treating means complete healing of the cancer, shown by an absence of clinical symptoms associated with the cancer. In a further aspect of the invention, treating means the mitigation of cancer or an associated disease condition or symptom by at least about 90% in the subject. In an additional aspect, treating means the mitigation of cancer or an associated disease condition or symptom by at least about 95% in the subject.

The methods of the invention include also methods of prolonging survival of a subject having cancer comprising administering therapeutically effective-amounts of the CAR T cells of the present invention and/or the inhibitors of Cathepsin B to a subject having cancer. The CAR T cells and the inhibitors of Cathepsin B may be administered in any order, separately or in combination, sequentially or concurrently, with overlapping or non-overlapping periods of administration. The methods of the invention thus include methods of prolonging survival of a subject having cancer, comprising concurrently administering therapeutically effective amounts of the CAR T cells and the inhibitors of Cathepsin B to a subject having cancer. The methods of the invention thus also include methods of prolonging survival of a subject having cancer, comprising sequentially administering therapeutically effective amounts of the CAR T cells and the inhibitors of Cathepsin B to a subject having cancer.

The term “prolonging survival” means extending the life span of a subject having cancer by at least one day versus a subject having the same cancer that does not receive the CAR T cells of the present invention. Prolonged survival includes increasing the life span of the subject by at least: 1, 2, 3, 4 or more weeks, 1, 2, 3, 4, 5, 6, 7, 8, 9, 10, 11, 12 or more months, or 1, 2, 3, 4, 5, or more years.

The amount of the CAR T cells and the inhibitors of Cathepsin B sufficient to have an effect on cancer (additive, synergistic or otherwise) in a subject will vary, for example, in view of the identity of the CAR T cells and the inhibitors of Cathepsin B being used in the combination, the physical characteristics of the subject, the severity of the subject's symptoms, the form of the cancer, the identity of the cancer, the formulations and means used to administer the drugs, and the method being practiced. The specific dose for a given subject is usually set by the judgment of the attending physician.

Depending on the means of administration, the dose may be administered all at once, or slowly over a period of time, such as with an intravenous administration. For slower means of administration, the administering period can be a matter of minutes, such as about 5, 10, 15, 20, 25, 30, 35, 40, 45, 50, 55, 60, 65, 70, 75, 80, 85, 90, 95, 100, 105, 110, 115, 120 or more minutes, or a period of hours, such as about 0.5, 1, 1.5, 2, 2.5, 3, 3.5, 4, 4.5, 5 or more hours. The administration of the dose may be interrupted, such as where the dose is administered via intravenous infusion and the dose is divided into two or more infusion bags. Under such circumstances, the administration of the dose may be interrupted while the infusion bags are changed.

As used herein, the terms “dose”, “unit dose”, “dosage”, “effective dose” and related terms refer to physically discrete units that contain a predetermined quantity of active ingredient or therapeutic agent calculated to produce a desired therapeutic effect.

As used herein, a “subject” is a human, a non-human primate, bird, horse, cow, goat, sheep, a companion animal, such as a dog, cat or rodent, or other mammal.

Exemplary cancers that may be treated via the methods of the invention also include, but are not limited to, leukemias including acute leukemias, such as acute myeloid leukemia (AML) and acute lymphoid leukemia (ALL; including B cell ALL (B-ALL) and T cell ALL (T-ALL)), chronic leukemias, such as chronic myeloid leukemia (CML) and chronic lymphoid leukemia (CLL); lymphoma; myeloma; sarcoma; non-small cell lung cancer; pancreatic cancer; gastric cancer; liver cancer; osteosarcoma; lung cancer; cervical cancer; colorectal cancer; breast cancer; prostate cancer; and all other hematologic malignancies and solid cancers including brain cancer.

### Additive and Synergistic Effects

In many instances, the combination of CAR T cells and the inhibitors of Cathepsin B taught herein may have an additive therapeutic effect on a cancer. However, the combinations may also or alternatively have a synergistic therapeutic effect on the cancer. Synergistic therapeutic effects are those that are substantially greater than what is seen when cancer cells are treated with either agent alone.

### IV. Examples

### 1. Characterization of CAR T Cell-Mediated Trogocytosis and its Implications.

### 1A. Functional and Molecular Characterization of CAR T Cell-Mediated Trogocytosis.

Flow cytometric analysis of target antigen transfer to CAR T cells from clinical patients. 7 days after CAR T cell injection, whole blood was isolated from patients receiving CD19 CAR T cell therapy for B cell lymphoma or B cell leukemia. Peripheral blood mononuclear cells (PBMCs) were isolated from whole blood using Ficoll-Paque. Next, PBMCs were stained with anti-hCD3, anti-hCD4, anti-hCD8, anti-CAR, and anti-hCD19, anti-hCD27, anti-hCD137, and 7-AAD and analyzed via flow cytometry. CD19 levels were assessed on CAR T cells. Two patients were found that showed significantly more CD19 on their CAR T cells compared to normal T cells (FIG. 2A), indicating that these CD19 CAR T cells underwent trogocytosis, resulting in the presence of CD19+ CAR T cells.

Assessment of fratricide in CD19-targeting CAR T cells. CD19-targeting CAR T cells or ΔscFv CAR T cells (CAR T cells lacking an antigen-recognition domain) were counted and diluted to a concentration of 1e6 cells per 1 mL. Next, 100 μL of CAR T cells were added to wells of a 96-well round bottom plate. Next, 100 μL of the CD19-expressing B cell lymphoma line Raji expressing luciferase (Raji-luc) were added to the appropriate wells at the following target-effector ratios: 2:1, 0.5:1, and 0:1 and incubated at 37° C./5% CO₂. After 24 hours, cells were washed with 1×PBS containing 2% BSA and stained with Zombie Red viability dye and anti-hCD3. Live cells were detected using Zombie Red viability dye. Cells were acquired in a BD LSR II. Cell counts were normalized according to the total volume acquired and the number of live CD3+ cells per condition was quantified using FlowJo. The results shown in FIG. 2B show that CD19-targeting CAR T cells have fewer live cell counts after coculture, indicative of CAR T cell death, potentially resulting from fratricide.

Determination of phenotype and exhaustion in CD19-targeting CAR T cells compared to ΔscFv CAR T cells. CD19-targeting CAR T cells or ΔscFv CAR T cells will be counted and diluted to a concentration of 1e6 cells per 1 mL. Next, 100 μL of CAR T cells will be added to wells of a 96-well round bottom plate. Next, 100 μL of cells of the CD19-expressing B cell lymphoma line Raji expressing luciferase (Raji-luc) will be added to the appropriate wells at the following target-effector ratios: 2:1, 1:1, 0.5:1, 0.25:1, 0.125:1, and 0:1 and incubated for 24 hours at 37° C./5% CO₂. The next day, cells will be washed with 1×PBS containing 2% BSA and stained with the following antibody cocktail: anti-hPD-1/PE, anti-hCD4/BUV496, anti-HA/APC, anti-hCD45RA/PE-Cy7, anti-hCD19/BV711, anti-hCD62L/BUV395, anti-hCD3/PerCP, anti-hTIM-3/BV421, anti-hLAG-3/AF700, anti-hCD95/BV510. Live cells will be detected using Zombie Red viability dye. Cells will be acquired in a 4-laser Cytek Aurora UV. Phenotype and exhaustion will be determined by assessing expression levels of CD45RA, CD62L, TIM-3, and LAG-3 on CAR T cells.

### 1B. Development of Luciferase Complementation Assay for Real-Time Assessment of CAR-Mediated Trogocytosis.

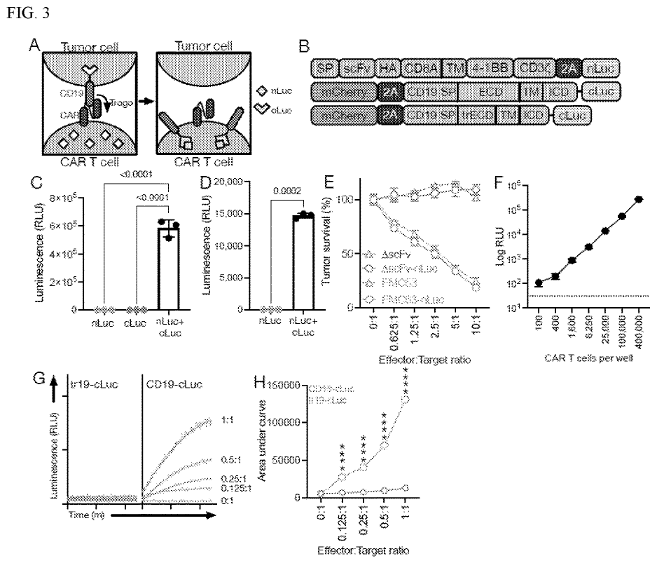

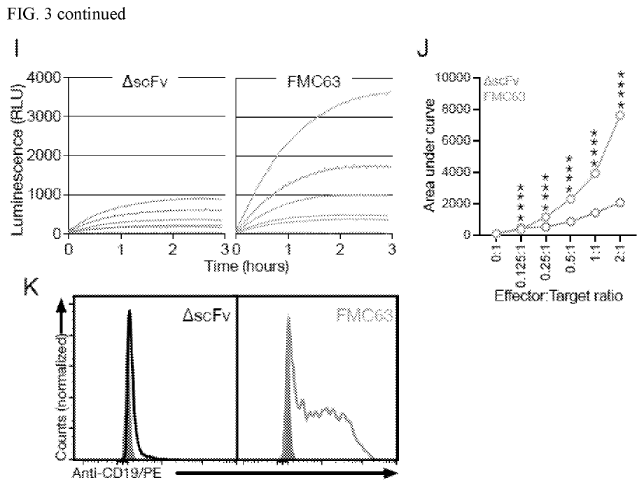

Confirmation of luciferase complementation in CAR T cells. A split luciferase pair (nLuc, cLuc) was identified with sub-nanomolar affinity [16] derived from the small and highly sensitive luciferase enzyme NanoLuc, allowing for spontaneous complementation when present in the same cell (FIG. 3A). CD19 CAR T cells were produced expressing either nLuc only or nLuc and cLuc together, with constructs used for production of nLuc expressing CAR T cells, CD19-cLuc expressing tumor cells, and tr19-cLuc-expressing cells shown in FIG. 3B (nLuc: nucleic acid sequence—SEQ ID NO:1, amino acid sequence—SEQ ID NO:2; cLuc: nucleic acid sequence—SEQ ID NO:3, amino acid sequence—SEQ ID NO:4; tr19-cLuc: nucleic acid sequence—SEQ ID NO:5, amino acid sequence—SEQ ID NO:6).

Next, a determination of luciferase complementation following transient transfection was performed via plate-based luminescence. 293T cells were transiently transfected with nLuc, cLuc, or both nLuc and cLuc. Cells were transiently transfected using Lipofectamine 2000 and incubated at 37 C/5% CO₂ for 24 hours. Following production, CAR T cells were plated at 25,000 CAR T cells per well. Next, Furimazine Live Cell Substrate (Promega) was diluted according to the manufacturer's protocol and added to all wells. The plate was incubated at 37° C./5% CO₂ for 5 minutes, then luminescence was measured using a Spark multi-mode plate reader (Tecan). Luminescence was only detectable when both nLuc and cLuc fragments are present together in CAR T cells, demonstrating luciferase complementation is possible when cells contain both nLuc and cLuc (FIG. 3C).

Primary human T cells isolated from healthy peripheral blood mononuclear cells (PBMCs) were transduced with gamma retrovirus encoding genes for CAR-2A-nLuc or CAR-2A-nLuc and tr19-cLuc. Cells were expanded for one week following transduction. It was demonstrated in primary human T cells that luciferase complementation is possible when cells contain both nLuc and cLuc. Presence of nLuc alone results in minimal luminescence (FIG. 3D).

Assessing cytotoxicity of nLuc-expressing CAR T cells. Next, it was verified that addition of the nLuc fragment to the CAR plasmid did not negatively impact CAR T cell function by assessing the ability of nLuc-expressing CAR T cells to kill tumor cells via plate-based luminescence. Cells of the CD19-positive Burkitt's lymphoma cell line Raji expressing firefly luciferase (Raji-luc) were plated in wells of a 96-well plate at 30,000 cells per well. Next, conventional CAR T cells (FMC63) or T cells expressing a CAR without a binding domain (ΔscFv) were compared to nLuc-expressing versions of FMC63 cells and ΔscFv T cells by adding the cells to the appropriate wells at the effector-target ratios indicated in FIG. 3E. The plate was incubated overnight at 37° C./5% CO₂. After 16 hours, cells were transferred to a black 96-well flat bottom plate. 2× D-luciferin was added to all wells at a 1:1 ratio and the plate was incubated at 37° C./5% CO₂ for 5 minutes. Luminescence was measured using a Spark multi-mode plate reader (Tecan). As shown in FIG. 3E, it was confirmed that addition of nLuc does not impact CAR T cell cytotoxicity against the CD19+ B cell lymphoma cell line Raji.

Titration of nLuc+ cLuc+ CAR T cells for the assessment of CompLuc sensitivity. CAR T cells expressing both nLuc and cLuc were produced by gamma retrovirus transduction. Following production, CAR T cells were serially diluted 1:4 to concentrations ranging from 400,000 CAR T cells per 100 μL to 100 CAR T cells per 100 μL. Next, 100 μL of CAR T cells were plated in triplicate into wells of a black 96 well plate. Next, optimized Furimazine Live Cell Substrate (Promega) was diluted 1:20 according to the manufacturer's protocol and 25 μL was added to each well. Cells were incubated for 5 minutes at 37° C./5% CO₂, then luminescence was measured using a Spark multi-mode plate reader (Tecan). Results were quantified in GraphPad Prism. As shown in FIG. 3F, it was found that CompLuc is capable of detecting as few as 100 CAR T cells per well, demonstrating its high sensitivity and utility in the real-time detection of CAR-mediated trogocytosis.

Demonstration of trogocytosis via CompLuc. K562 tumor cells expressing CD19-cLuc or CD19-cLuc lacking the extracellular domain of CD19 (tr19-cLuc) were resuspended in Opti-MEM serum-free media at a concentration of 100,000 cells per 50 μL. 50 μL of K562 cells were added to wells of a black 96-well round bottom plate. Next, Live Cell Substrate (Promega N2011) was diluted 1:20 according to the manufacturer's protocol and 25 μL of prepared substrate was added to all wells. nLuc-expressing CAR T cells were resuspended in Opti-MEM serum free media and added to the prepared wells at the following effector-target ratios: 1:1, 0.5:1, 0.25:1, 0.125:1, or 0:1. Luminescence was measured every minute for a period of 2 hours. All values were quantified using GraphPad Prism and area-under-curve analysis. As shown in FIG. 3G, it was found that CompLuc allowed the quantification of trogocytosis in real-time with minimal background and levels of trogocytosis were directly dependent on the effector-target ratio. FIG. 3H is the area under curve analysis for total luminescence in FIG. 3G. Values were calculated using “area under curve” analysis in GraphPad Prism 10. **** indicates that p<0.0001. These results demonstrate that trogocytosis is dependent on the number of CAR T cells present, where more CAR T cells correlates to increased trogocytosis. Trogocytosis is dependent on the interaction between the CAR and the external domain of the tumor antigen, as CAR T cells cocultured with tr19-cLuc-expressing tumor cells did not exhibit luminescence.

Kinetic assessment of CAR-mediated trogocytosis using CompLuc. nLuc expressing CAR T cells (FMC63 [blue] or ΔscFv [grey]) were cocultured with CD19-cLuc-expressing K562 cells for 3 hours at the following effector-target ratios: 2:1, 1:1, 0.5:1, 0.25:1, 0.125:1, 0:1. CAR T cells were resuspended in Opti-MEM serum-free media at a concentration of 100,000 cells per 50 μL. 50 μL of K562 cells were added to wells of a black 96-well round bottom plate. Next, Live Cell Substrate (Promega N2011) was diluted 1:20 according to the manufacturer's protocol and 25 μL of prepared substrate was added to all wells. nLuc-expressing CAR T cells were resuspended in Opti-MEM serum free media and added to the prepared wells at the following effector-target ratios: 1:1, 0.5:1, 0.25:1, 0.125:1, or 0:1. Luminescence was measured every minute for a period of 3 hours. All values were quantified using GraphPad Prism and area-under-curve analysis. The results are provided in FIG. 3I and they demonstrate that the luciferase complementation assay is dependent on the specific interaction between the extracellular domain of the tumor antigen with the binding domain of the CAR. FIG. 3J shows the area under curve analysis for total luminescence in FIG. 3I. Values were calculated using “area under curve” analysis in GraphPad Prism 10. **** indicates that p<0.0001.

Determination of CD19 transfer to CAR T cells as measured by flow cytometry. ΔscFv or FMC63 CAR T cells were cocultured with CD19-cLuc-expressing K562 tumor cells at a 0.25:1 effector-target ratio for one hour prior to analysis. Following coculture, cells were placed on ice, washed with 1×PBS containing 2% bovine serum albumin (FACS buffer), and centrifuged for 5 minutes at 400 xg. After washing, cells were stained with anti-CAR, anti-hCD3, anti-hCD19, and a cell viability dye according to manufacturer's instructions. After staining, cells were washed with FACS buffer and centrifuged for 5 minutes at 400 xg. Cells were resuspended in 400 μL 1×PBS and acquired in a Beckton Dickinson LSRII flow cytometer. The results are provided in FIG. 3K and they validate the results shown in FIG. 3I,J, demonstrating that CD19 transfer to CAR T cells is dependent on the specific interaction between the extracellular domain of the tumor antigen with the binding domain of the CAR. Importantly, luminescence correlates to the presence of tumor antigen on the CAR T cell surface.

### 1C. Identification of Key Modulators of CAR-Mediated Trogocytosis.

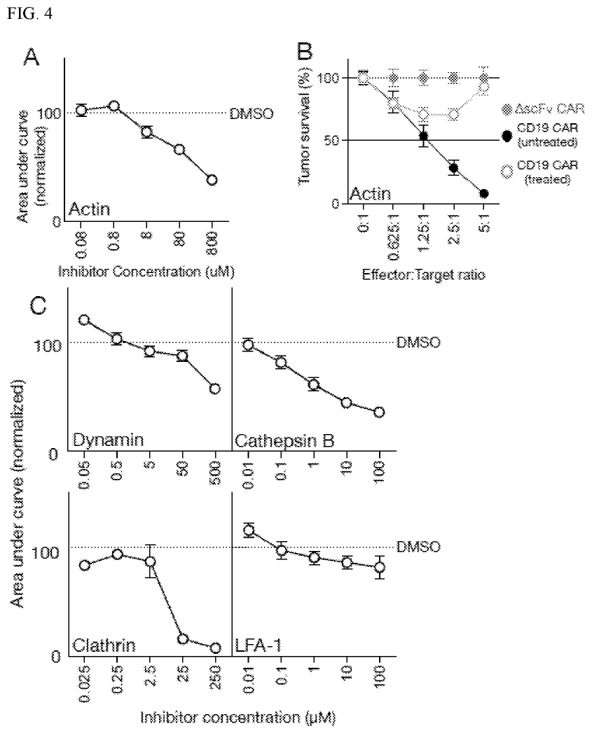

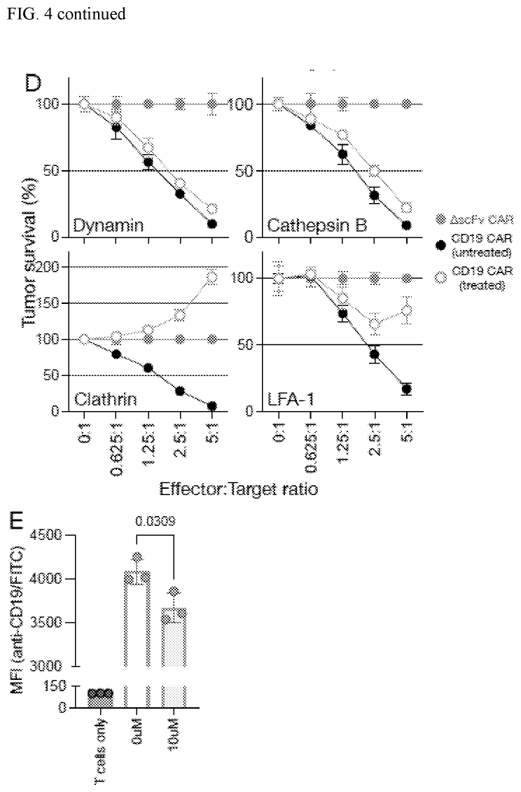

Inhibition of a known modulator of trogocytosis for the validation of CompLuc. Actin polymerization has been previously shown to have a role in trogocytosis. Specifically, inhibition of actin polymerization leads to a reduction in trogocytosis [6,17]. To validate the utility of CompLuc, trogocytosis was measured following treatment of nLuc expressing CAR T cells (FMC63) with the actin polymerization inhibitor Cytochalasin D. CAR T cells were treated for one hour with one of the following concentrations of Cytochalasin D17: 800 μM, 80 μM, 8 μM, 0.8 μM, or 0.08 μM. CAR T cells treated with DMSO were used as an untreated control. Following pre-treatment, CAR T cells were washed with RPMI containing 10% Fetal Bovine Serum and 1% penicillin-streptomycin to remove any excess inhibitor. After washing, CD19-cLuc-expressing K562 tumor cells were added to wells of a black 96-well round-bottom plate at a concentration of 100,000 cells per 50 μL per well. Next, 25 μL of diluted Live Cell Substrate (Promega) was added to each well, followed by 50 μL of pre-treated CAR T cells at a 1:1 effector-target ratio. Immediately after adding CAR T cells to each well, luminescence was measured every minute for a period of three hours to assess trogocytosis. Results were quantified as area-under-curve in GraphPad prism and shown in FIG. 4A. Using CompLuc, it was found that treatment of CAR T cells with Cytochalasin D leads to a significant reduction in trogocytosis, validating CompLuc as a useful tool in assessing trogocytosis in real-time.

Killing of CD19-positive Raji-luc cells by FMC63 CAR T cells treated with 80 μM of Cytochalasin D. nLuc expressing CAR T cells (FMC63) were co-cultured with Raji-luc cells at the indicated effector-target ratios and incubated for 16 hours at 37 C/5% CO₂. Excess inhibitor was washed off prior to coculture. CAR T cells were treated for one hour with 80 μM of Cytochalasin D. CAR T cells treated with DMSO were used as an untreated control. Following pre-treatment, CAR T cells were washed with RPMI containing 10% Fetal Bovine Serum and 1% penicillin-streptomycin to remove any excess inhibitor. CAR T cells were cocultured with Raji-luc cells at 5:1, 2.5:1, 1.25:1, 0.625:1, and 0:1 effector-target ratios and incubated for 16 hours. To assess cytotoxicity, cells were transferred to a black 96-well flat bottom plate. Next, 2× D-luciferin was added to all wells at a 1:1 ratio and the plate was incubated at 37° C./5% CO₂ for 5 minutes. Luminescence was measured using a Spark multi-mode plate reader (Tecan). The results provided in FIG. 4B show that treatment of CAR T cells with Cytochalasin D results in a loss of CAR T cell cytotoxicity, demonstrating that inhibition of trogocytosis by this approach is not compatible with therapeutic function.

Assessment of trogocytosis following the inhibition of various key immune synapse processes. It was hypothesized that the occurrence of CAR-mediated trogocytosis could be reduced by inhibiting different proteins involved in immune synapse formation and function. To this end, the following targets were selected: Dynamin, Clathrin, Cathepsin B, and LFA-1. nLuc-expressing FMC63 CAR T cells were pre-treated with one of the following inhibitors for one hour: Dynasore (Dynamin) [18], Ca-074-Me (Cathepsin B) [19,20], Pitstop (Clathrin), or BI-1950 (LFA-1). After treatment, excess inhibitor was washed away using RPMI containing 10% fetal bovine serum. Next, treated CAR T cells were cocultured with CD19-cLuc-expressing K562 cells at a 1:4 effector-target ratio. Trogocytosis was assessed using CompLuc as previously described. Results were quantified as area-under-curve in GraphPad prism and shown in FIG. 4C. It was found that inhibition of Clathrin and Cathepsin B resulted in a significant reduction in trogocytosis at low or intermediate treatment concentrations; inhibition of Dynamin and LFA-1 only saw a modest reduction in trogocytosis. These data show Clathrin and Cathepsin B as potential targets for the reduction of CMT.

Assessment of cytotoxicity following the inhibition of various key immune synapse processes. Next, cytotoxicity of CD19 CAR T cells was compared against the CD19+ B cell lymphoma cell line Raji expressing firefly luciferase (Raji-luc) using plate-based luminescence. CAR T cells were pre-treated with one of the following inhibitors: Dynasore (Dynamin, 50 μM), Ca-074-Me (Cathepsin B, 10 μM), Pitstop (Clathrin, 25 μM), or BI-1950 (LFA-1, 10 μM) for one hour prior to coculture. CAR T cells treated with DMSO were used as an untreated control. Raji-luc cells were plated in wells of a 96-well round bottom plate at 30,000 cells per well. CAR T cells were added to wells at the following effector-target ratios: 10:1, 5:1, 2.5:1, 1.25:1, 0.625:1, and 0:1. Plates were incubated at 37° C./5% CO₂ for 16 hours. To assess cytotoxicity, cells were transferred to a black 96-well flat bottom plate. Next, 2× D-luciferin was added to all wells at a 1:1 ratio and the plate was incubated at 37° C./5% CO₂ for 5 minutes. Luminescence was measured using a Spark multi-mode plate reader (Tecan). It was found that inhibition of Dynamin and Cathepsin B did not result in a substantial loss in CAR T cell cytotoxicity compared to the untreated control, whereas inhibition of Clathrin and LFA-1 resulted in impaired cytotoxicity (FIG. 4D). Cathepsin B is the only modulator found to reduce trogocytosis without compromising CAR T cell cytotoxicity. Therefore, Cathepsin B was selected as a potential key modulator of CMT for further exploration.

Validation of CompLuc findings for selected modulators via flow cytometry. To validate the CompLuc findings, CD19 transfer to CAR T cells was assessed via flow cytometry. First, CAR T cells were treated with 10 μM of the Cathepsin B inhibitor Ca-074-Me or DMSO for one hour. Next, 100,000 CD19-cLuc-expressing K562 cells were added to wells of a 96-well round bottom plate. CAR T cells treated with DMSO or Ca-074-Me were added to the appropriate wells at a 1:4 effector-target ratio. Plates were incubated at 37° C./5% CO₂ for one hour. Following the incubation, cells were washed with 1×PBS containing 2% BSA, stained with anti-hCD19/FITC, and acquired via flow cytometry. Cells were analyzed in FlowJo and mean fluorescence intensity (MFI) was used to assess CD19 transfer. It was found that treatment with Ca-074-Me led to a significant decrease in CD19 transfer to CAR T cells (FIG. 4E), demonstrating that inhibition of Cathepsin B reduces CAR-mediated trogocytosis.

### 2. Assessment of Cystatin a Overexpression on CAR T Cell Trogocytosis and Function.

The data presented above suggested that inhibition of the cysteine protease Cathepsin B (CTSB) leads to a reduction in trogocytosis, as measured by CompLuc and flow cytometry. CTSB is a ubiquitously expressed cysteine protease primarily localized to the endosomal and lysosomal compartments [21-24]. CTSB^{KO} mice are both viable and fertile, exhibiting no phenotypic differences compared to CTSB^{WT} mice [22], suggesting that therapeutic targeting of CTSB is safe. In addition to its role in proteolytic degradation within the endosome and lysosome, CTSB has been shown to degrade and remodel components of the extracellular matrix, including collagen, fibronectin, and laminin, thereby promoting the invasion and migration of tumor cells [25,26]. In T lymphocytes, CTSB has been shown to line cytotoxic granules and to localize to the immune synapse [23].

Due to the broad expression of CTSB across most tissues [22-24], systemic inhibition of CTSB using small molecule inhibitors may be inefficient and/or result in toxicities [27,28]. Ideally, CTSB inhibition could be could achieved exclusively within CAR T cells to minimize potential toxicity and increase efficacy, for example by expressing an endogenous inhibitor of CTSB. Using an endogenous human protein to inhibit CTSB also has the advantage of being well-tolerized and not causing unintended immune reactivity. Cystatin A is a human protein that, similar to Ca-074-Me, occludes the active site of CTSB [29-31]. Therefore, it was explored whether overexpression of Cystatin A could be used to specifically reduce trogocytosis by inhibiting CTSB.

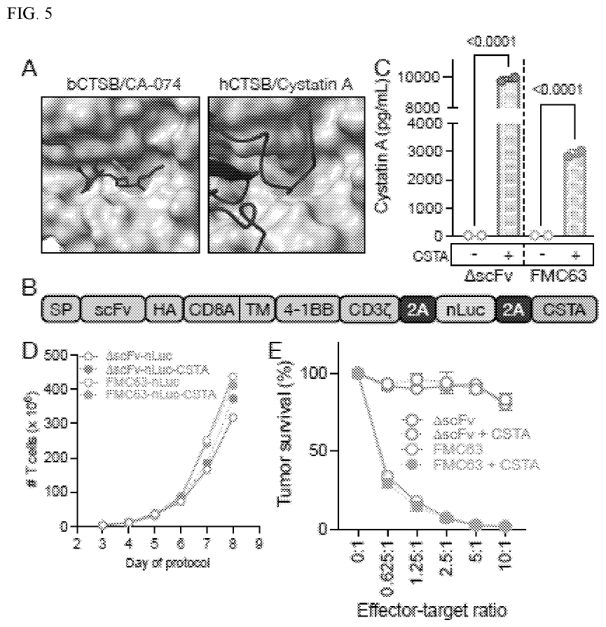

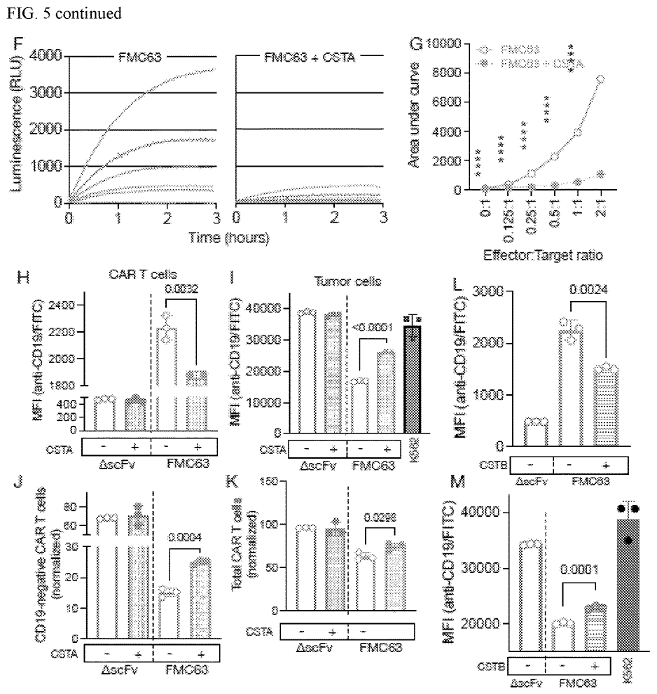

The crystal structures of bovine Cathepsin B (bCTSB) in complex with Ca-074 is shown in FIG. 5A (left, PDB: 1QDQ) and human Cathepsin B (hCTSB) in complex with Cystatin A (right, PDB: 3K9M). The active site cysteine of Cathepsin B is shown in pink; Ca-074 and Cystatin A are shown in blue. This demonstrates that the human protein Cystatin A inhibits Cathepsin B in the same way as the small molecule inhibitor Ca-074.

### 2A. Assessment of Cystatin a Overexpression on CAR T Cell Trogocytosis and Function.

Generation of Cystatin A-overexpressing CD19 CAR T cells. Cystatin A-overexpressing CD19 CAR T cells were produced by gamma retrovirus transduction as previously described [44]. FIG. 5B shows the construct used for the production of Cystatin A-overexpressing, nLuc expressing CAR T cells, where Cystatin A is separated from the CAR by a 2A sequence (FMC63-2A-nLuc-2A-CSTA: nucleic acid sequence—SEQ ID NO:7, amino acid sequence—SEQ ID NO:8).

Assessment of Cystatin A overexpression via ELISA. Following the generation of Cystatin A-overexpressing CD19 CAR T cells, the overexpression of Cystatin A was assessed using a commercially available Cystatin A ELISA kit (Thermofisher #EH140RB) to show Cystatin A could be overexpressed without compromising CAR T cell expansion. Lysates from ΔscFv and FMC63 CAR T cells were incubated in transparent 96-well flat bottom plates coated with anti-human Cystatin A for 2.5 hours. Following the incubation, plates were washed 5 times with 1×PBS containing 0.5% Tween. Next, plates were incubated for one hour with diluted biotinylated anti-human Cystatin A. Following this incubation, the wash steps were repeated, and plates were incubated for one hour with diluted Streptavidin-HRP. After this incubation, the plates were washed and incubated with 3,3′,5,5′-Tetramethylbenzidine for 30 minutes protected from light. After 30 minutes, 1N H₂SO₄ was added to all wells to stop the reaction, and absorbance was measured at 450 nm. All experiments were performed in duplicate. As shown in FIG. 5C, it was confirmed that Cystatin A can be overexpress in CAR T cells with no effect on expansion.

Expansion of ΔscFv and FMC63 CAR T cells overexpressing Cystatin A during manufacturing, as determined by automated cell counting using a Thermofisher Countess II. 10 μL of CAR T cells was diluted in 10 μL of Trypan Blue dye (Invitrogen #T10282). As shown in FIG. 5D, Cystatin A overexpression does not impact CAR T cell expansion.

Examine the effects of Cystatin A overexpression on CAR T cell cytotoxicity. Overexpression of Cystatin A and subsequent Cathepsin B inhibition could result in changes to CAR T cell physiology and function. Therefore, it is imperative to verify that Cystatin A overexpression does not negatively impact the ability of CAR T cells to kill tumor cells. Therefore, cytotoxicity of CD19 CAR T cells was compared with and without Cystatin A overexpression against Raji-luc cells using plate-based luminescence. Raji-luc cells were plated in wells of a 96-well round bottom plate at 30,000 cells per well. CAR T cells were added to wells at the following effector-target ratios: 10:1, 5:1, 2.5:1, 1.25:1, 0.625:1, and 0:1. Plates were incubated at 37° C./5% CO₂ for 16 hours. To assess cytotoxicity, cells were transferred to a black 96-well flat bottom plate. Next, 2× D-luciferin was added to all wells at a 1:1 ratio and the plate was incubated at 37° C./5% CO₂ for 5 minutes. Luminescence was measured using a Spark multi-mode plate reader (Tecan). As shown in FIG. 5E, it was found that overexpression of Cystatin A had no effect on the in-vitro cytotoxicity of CAR T cells.

Kinetic assessment of CAR-mediated trogocytosis using CompLuc assay. K562 tumor cells expressing CD19-cLuc were incubated with nLuc expressing CD19 CAR T cells or nLuc expressing CD19 CAR T cells overexpressing Cystatin A. K562 tumor cells and CAR T cells were prepared as described above in the section entitled Kinetic assessment of CAR-mediated trogocytosis using CompLuc. Optimized Live Cell Substrate (Promega N2011) was used in place of the substrate furimazine. Luminescence was measured every minute for 3 hours. As shown in FIG. 5F, Cystatin A overexpression results in a significant reduction in trogocytosis as measured by the luciferase complementation assay

Determine if Cystatin A overexpression results in reduced trogocytosis via CompLuc. To assess trogocytosis using the luciferase complementation assay, CAR T cells were cocultured with CD19-cLuc-expressing K562 cells. K562 cells were suspended in Opti-MEM reduced serum media at a concentration of 100,000 cells per 50 μL. K562 cells were plated in wells of a black 96-well round bottom plate at 100,000 cells per well. Next, Furimazine live cell substrate (Promega) was diluted 1:20 in dilution buffer per the manufacturer's instructions and added to all wells at 25 μL per well. Next, conventional or Cystatin A-overexpressing CAR T cells were added to the appropriate wells at the following effector-target ratios: 2:1, 1:1, 0.5:1, 0.25:1, 0.125:1, 0:1, 50 μL per well. Immediately following the addition of CAR T cells, luminescence was quantified every minute for 3 hours using a Spark multi-mode plate reader (Tecan), where luminescence correlates to increased trogocytosis. Area under curve was calculated using “area under curve” analysis in GraphPad Prism 10. Statistical significance was determined using unpaired t test. Significance was defined as p<0.05. **** indicates that p<0.0001. As shown in FIG. 5G, and using CompLuc, it was found that overexpression of Cystatin A in CAR T cells resulted in a near complete loss of luminescence, indicative of significantly reduced trogocytosis. This is consistent with the earlier data showing that inhibition of Cathepsin B using the small molecule inhibitor Ca-074-Me also reduces CAR-mediated trogocytosis.

Determine if Cystatin A overexpression results in reduced trogocytosis via flow cytometry. It was hypothesized that overexpression of cystatins will inhibit CTSB, thereby reducing trogocytosis. To assess this via flow cytometry, CAR T cells were stained with Cell Trace Far Red (Invitrogen) according to the manufacturer's instructions for gating purposes. Next, CAR T cells were cocultured in 96-well round bottom plates with CD19-cLuc-expressing K562 tumor cells at 2:1, 1:1, 0.5:1, 0:1, and 1:0 effector-target ratios. Plates were incubated for 30 minutes at 37° C./5% CO₂. Following incubation, cells were placed on ice and transferred to a 96-well V-bottom plate for staining. Cells were washed twice with 200 μL 1×PBS containing 2% BSA (FACS buffer) and centrifuged at 400×g for 5 minutes between wash steps. After washing, cells were stained with Zombie Violet viability stain (Invitrogen) and incubated on ice for 10 minutes. Following incubation, cells were stained with anti-CD19/FITC and incubated for 30 minutes at 4° C. Following incubation, cells were washed twice with 200 μL FACS buffer and centrifuged at 400×g for 5 minutes between wash steps. Lastly, cells were resuspended in 175 μL 1×PBS+25 μL Accucheck counting beads (Invitrogen) and transferred to tubes for acquisition. Results from flow cytometry were quantified in FlowJo as differences between Mean Fluorescence intensity of CD19 on CAR T cells. CAR T cell counts were normalized using Accucheck counting beads.

Results comparing trogocytosis in conventional and Cystatin-overexpressing CAR T cells were analyzed using a Student's t test and considered statistically significant if p<0.05. FIG. 5H—Transfer of CD19 to CAR T cells was determined via flow cytometry. The data shows Cystatin A overexpression results in significantly less CD19 transfer to CAR T cells as measured by flow cytometry. FIG. 5I—CD19 loss on tumor cells was determined via flow cytometry. The data shows Cystatin A overexpression significantly rescues CD19 expression on the surface of tumor cells as measured by flow cytometry. FIG. 5J—Percentage of CAR T cells negative for CD19 were quantified via flow cytometry. The data shows Cystatin A overexpression results in significantly more CD19-negative CAR T cells, which is important in the prevention of CAR T cell fratricide. FIG. 5K—Total CAR T cells were quantified via flow cytometry and normalized to ΔscFv. The data shows Cystatin A overexpression results in significantly more CAR T cells at the end of the coculture, indicative of reduced CAR T cell fratricide.

The experiment was repeated using CD19-cLuc-expressing K562 cells cocultured with CAR T cells or CAR T cells overexpressing Cystatin B for 30 minutes. FIG. 5L—CD19 transfer to CAR T cells was determined via flow cytometry. FIG. 5M—CD19 loss on K562 tumor cells was determined via flow cytometry.

The results demonstrate that overexpression of Cystatin A results in a significant reduction of CD19 transfer to CAR T cells as measured by flow cytometry, supporting the hypothesis that Cystatin A overexpression reduces trogocytosis.

Determine if Cystatin A overexpression reduces Cathepsin B activity in-vitro and results in minimal off-target effects. Whether Cystatin A overexpression leads to a reduction in Cathepsin B activity will be verified. To this end, Cathepsin B activity will be will measured using a commercially available Cathepsin B activity assay kit (Millipore Sigma) [32]. CAR T cell lysates will be prepared according to the kit instructions and Cathepsin B activity will be measured fluorometrically. Cystatin A has also been shown to interact with other proteins, such as Cathepsin L and Caspase 1 [33,34]. It is possible that overexpression of Cystatin A will result in inhibition of various different proteases in addition to Cathepsin B; therefore, the activity of Cathepsin L and Caspase-1 will be assessed in conventional and Cystatin A-overexpressing CAR T cells. To do this, commercially available Cathepsin L and Caspase-1 activity kits will be used. Similarly, CAR T cell lysates will be prepared according to the kit instructions and Cathepsin L activity and Caspase-1 activity will be determined fluorometrically and colorimetrically.

Determine if Cystatin A overexpression results in changes to CAR T cell phenotype following generation. Studies have shown that the phenotype of CAR T cells infused into patients correlates with CAR T cell persistence [35,36]. Therefore, a further aim is to assess the phenotype of CAR T cells with or without Cystatin A overexpression following production. To this end, CAR T cell phenotype will be assessed on the final day of production, prior to cryopreservation. 1e6 CAR T cells per condition will be isolated and washed with 1×PBS containing 2% BSA (FACS buffer) and centrifuged at 400×g for 5 minutes. Next, CAR T cells will be stained with the following antibodies: anti-hCD45RA, anti-hCD4, anti-hCD8, anti-hCD3, anti-CAR, anti-hCD62L, and anti-hCD45RO. Cells will be incubated for 30 minutes at 4° C. Following incubation, cells will be washed with FACS buffer and centrifuged at 400×g for 5 minutes. Cells will be resuspended in 400 uL 1×PBS and analyzed via flow cytometry. CAR T cell counts will be normalized using Accucheck counting beads. MFI of exhaustion and memory-stem-like markers will be calculated using FlowJo software.

### 2B. Comparison of Long-Term CAR T Cell Function and Persistence in Conventional and Cystatin A-Overexpressing CAR T Cells.

Comparison of phenotype and persistence of conventional or Cystatin-overexpressing CAR T cells in-vitro. A serial coculture experiment will be performed to assess the long-term persistence and phenotype of conventional and Cystatin A-overexpressing CAR T cells. CAR T cells will be cocultured with the CD19+ B cell lymphoma cell line Toledo expressing firefly luciferase (Toledo-luc) at a 0.25:1 effector-target ratio in wells of a 96-well round bottom plate. Plates will be incubated for 48 hours at 37° C./5% CO₂. After 48 hours, 100 μL of cells will be mixed with 100 μL of 2× D-luciferin and incubated at 37° C./5% CO₂ for 5 minutes. Luminescence will be measured to assess cytotoxicity. Following luminescence reading, remaining CAR T cells will be counted and renormalized to the proper effector-target ratios, and fresh Toledo cells will be added to the wells. Plates will be incubated for another 48 hours at 37° C./5% CO₂. This process will repeat until there is a noticeable change in luminescence, indicative of reduced CAR T cell function. At this stage, cells will be washed with 1×PBS containing 2% BSA and stained with the following antibody cocktail: anti-hPD-1/PE, anti-hCD4/BUV496, anti-HA/APC, anti-hCD45RA/PE-Cy7, anti-hCD19/BV711, anti-hCD62L/BUV395, anti-hCD3/PerCP, anti-hTIM-3/BV421, anti-hLAG-3/AF700, anti-hCD95/BV510 [39-42]. Cells will be acquired in a 4-laser Cytek Aurora UV and analyzed in FlowJo. Live cells will be quantified using Zombie Red viability dye. Phenotype and exhaustion will be determined by assessing expression levels of CD45RA, CD62L, TIM-3, and LAG-3 on CAR T cells [6,43].

In-vivo assessment of CAR T cell efficacy and tumor control. It is hypothesized that a reduction in trogocytosis and a resulting increase in CAR T cell persistence will increase the in-vivo efficacy of CAR T cells. To assess this, a well-established in-vivo tumor model will be used [5,6] using the CD19-positive B cell leukemia cell line NALM6 expressing firefly luciferase (NALM6-luc) to determine the effect of Cystatin A overexpression on CAR T cell persistence in-vivo. 10-week-old NOD.Cg-Prkd^{scid} Il2rg^{tm1wjl}/SzJ (NSG) mice will undergo sublethal irradiation, followed by intravenous injection of 0.4e6 NALM6-luc cells via tail vein injection. Four days after tumor cell injection, mice will be injected with 1e6 conventional or Cystatin A-overexpressing CAR T cells via tail vein injection. Mice will be anesthetized, and tumor burden will be assessed using an in-vivo imaging system (IVIS). Tumor burden will be assessed via IVIS immediately prior to CAR T cell injection and weekly following CAR T cell injection. Tumor burden will be analyzed in Living Image by Perkin Elmer. To determine statistical significance of differences in tumor size between treatment groups, a two-sided Student's t test will be used and results will be considered significant if p<0.05. Statistical significance of differences in survival of mice treated with conventional and Cystatin A-overexpressing CAR T cells will be determined via log-rank test and Kaplan-Meier curves. Results will be considered significant if p<0.05.

Quantification of conventional or Cystatin-overexpressing CAR T cells in murine bone marrow and spleen. It is hypothesized that by reducing trogocytosis, CAR T cell fratricide can be reduced and therefore CAR T cell persistence improved in-vivo. CAR T cell persistence will be assessed using the in-vivo protocol described in above (“In-vivo assessment of CAR T cell efficacy and tumor control”). Three weeks after CAR T cell injection, mice will be sacrificed, and spleens and bone marrow will be harvested and analyzed via flow cytometry. To assess CAR T cell persistence, samples will be stained with anti-CAR, anti-hCD3, anti-hCD19, anti-hCD45, and anti-mCD45. Volumes of samples will be normalized using Accucheck counting beads (Invitrogen) and CAR T cell numbers (CAR+CD3+hCD45+mCD45−) will be quantified using FlowJo software. To determine statistical significance between differences in CAR T cell numbers, a two-sided Student's t test will be used and results will be considered significant if p<0.05.

In-vivo assessment of CAR T cell exhaustion and CD19 expression on tumor cells and conventional or Cystatin-overexpressing CAR T cells. CAR T cell exhaustion is a major roadblock in long-term persistence in patients. Therefore, following the in-vivo experiment described in above (“In-vivo assessment of CAR T cell efficacy and tumor control”), CAR T cell exhaustion will also be assessed in conventional and Cystatin-overexpressing CAR T cells. To determine CAR T cell phenotype, cells will be stained with anti-CAR, anti-hCD19, anti-mCD45, anti-hCD45, anti-hPD-1, anti-hLAG-3, and anti-hTIM-3 [6,43]. MFI of exhaustion markers and CD19 will be calculated using FlowJo software. To determine statistical significance of differences of MFIs and percentages of cells between conventional and Cystatin A-overexpressing CAR T cells, a two-sided Student's t test will be used and results will be considered significant if p<0.05.

### 3. Assessment of Truncated Cystatin A (CSTA₁₋₅₇) or Cystatin B Overexpression on CAR T Cell Trogocytosis and Function.

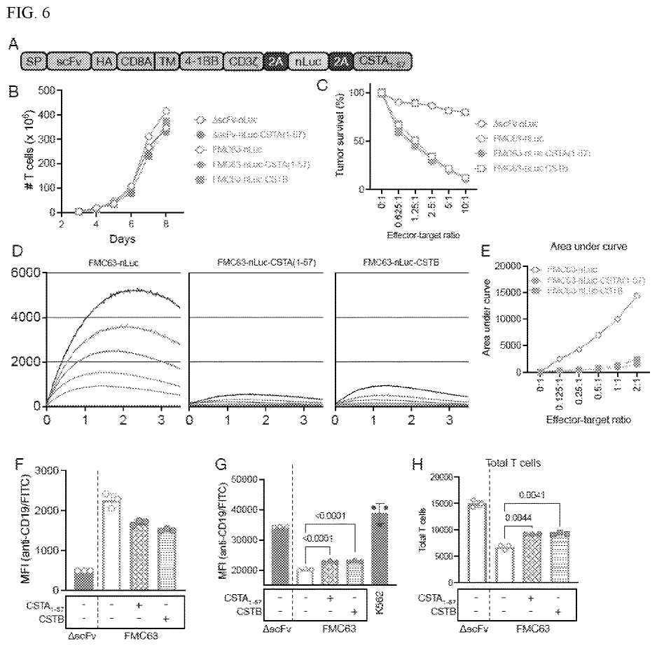

FIG. 6A shows the construct used for the production of Cystatin A₁₋₅₇-overexpressing, nLuc expressing CAR T cells (FMC63-2A-nLuc-2A-CSTA₁₋₅₇: nucleic acid sequence—SEQ ID NO: 11; amino acid sequence—SEQ ID NO:12). nLuc-expressing CAR T cells overexpressing Cystatin B were also produced (FMC63-2A-nLuc-2A-CSTB: nucleic acid sequence: SEQ ID NO:9; amino acid sequence—SEQ ID NO:10).

Expansion of ΔscFv and FMC63 CAR T cells overexpressing Cystatin A during manufacturing, as determined by automated cell counting using a Thermofisher Countess II. 10 μL of CAR T cells was diluted in 10 μL of Trypan Blue dye (Invitrogen #T10282). The results shown in FIG. 6B show that overexpression of truncated Cystatin A (CSTA₁₋₅₇) or Cystatin B (CSTB) does not impact CAR T cell expansion.

Killing of Raji-luc cells by FMC63 or ΔscFv CAR T cells with or without overexpression of Cystatin A, as determined by luminescence and described in the section entitled Titration of nLuc+ cLuc+ CAR T cells for the assessment of CompLuc sensitivity. CAR T cells were cocultured with Raji cells at the indicated effector-target ratios and incubated for 16 hours at 37 C/5% CO₂. To assess cytotoxicity, cells were transferred to a black 96-well flat bottom plate. Next, 2× D-luciferin was added to all wells at a 1:1 ratio and the plate was incubated at 37° C./5% CO₂ for 5 minutes. Luminescence was measured using a Spark multi-mode plate reader (Tecan). The results shown in FIG. 6C show that overexpression of truncated Cystatin A (CSTA₁₋₅₇) or Cystatin B (CSTB) does not impact CAR T cell cytotoxicity.

Kinetic assessment of CAR-mediated trogocytosis using CompLuc assay as described in sections entitled Kinetic assessment of CAR-mediated trogocytosis using CompLuc and Determination of CD19 transfer to CAR T cells as measured by flow cytometry. K562 tumor cells expressing CD19-cLuc were incubated with nLuc expressing CD19 CAR T cells or nLuc expressing CD19 CAR T cells overexpressing Cystatin A(₁₋₅₇)- or Cystatin B. Optimized Live Cell Substrate (Promega N2011) was used in place of the substrate furimazine. Luminescence was measured every minute for 3 hours. The results shown in FIG. 6D show that overexpression of truncated Cystatin A (CSTA₁₋₅₇) or Cystatin B (CSTB) results in a significant reduction in CAR T cell trogocytosis as measured by CompLuc, similar to data shown in FIG. 5F.

FIG. 6E sows the area under curve analysis for total luminescence in FIG. 6D. Area under curve was calculated using “area under curve” analysis in GraphPad Prism 10.

CD19-cLuc-expressing K562 cells were cocultured with CAR T cells or CAR T cells overexpressing Cystatin A at a 1:1 effector-target ratio for 30 minutes. K562 tumor cells and CAR T cells were counted using a Thermofisher Countess II Automated cell counter. CAR T cells were stained with Cell Trace Far Red (Invitrogen #C34564) according to manufacturer's instructions. Cells were washed with RPMI media supplemented with 10% fetal bovine serum and 1% penicillin/streptomycin, centrifuged for 5 minutes at 400 xg, and resuspended to a concentration of 2e6 cells/mL. 100 μL of tumor cells and 100 μL of CAR T cells were added to wells of a 96-well plate and incubated for 30 minutes at 37° C./5% CO₂. Following incubation, cells were placed on ice, washed with 200 μL FACS buffer, and centrifuged for 5 minutes at 400 xg. Cells were then stained with anti-hCD19 and a fixable viability stain. After staining, cells were washed with FACS buffer and centrifuged for 5 minutes at 400 xg. Cells were resuspended in 400 μL 1×PBS and acquired in a Beckton Dickinson LSRII flow cytometer. (F) Transfer of CD19 to CAR T cells was determined via flow cytometry. (G) CD19 loss on tumor cells was determined via flow cytometry. (H) Total CAR T cells were quantified via flow cytometry. The results shown in FIG. 6F show that Cystatin A (CSTA₁₋₅₇) or Cystatin B (CSTB) overexpression results in significantly less CD19 transfer to CAR T cells as measured by flow cytometry. The results shown in FIG. 6G show that Cystatin A (CSTA₁₋₅₇) or Cystatin B (CSTB) overexpression significantly rescues CD19 expression on the surface of tumor cells as measured by flow cytometry. The results shown in FIG. 6H show that Cystatin A (CSTA₁₋₅₇) or Cystatin B (CSTB) overexpression results in significantly more CAR T cells at the end of the coculture, indicative of reduced CAR T cell fratricide. All data shown represents mean of three values ±s.d. All statistical significance was determined using unpaired t-test. Significance was defined as p<0.05. * indicates p<0.05; ** indicates p<0.01; *** indicates p<0.001; **** indicates that p<0.0001.

### 4. Development of a Novel System for the Degradation of Trogocytosed Antigen.

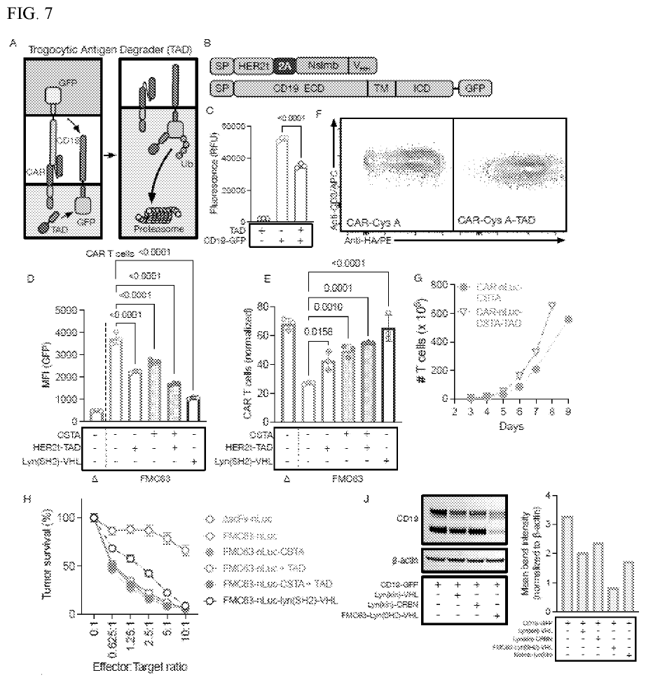

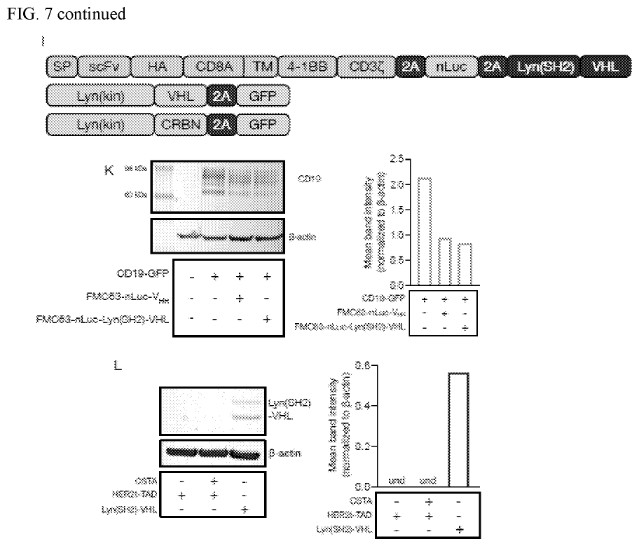

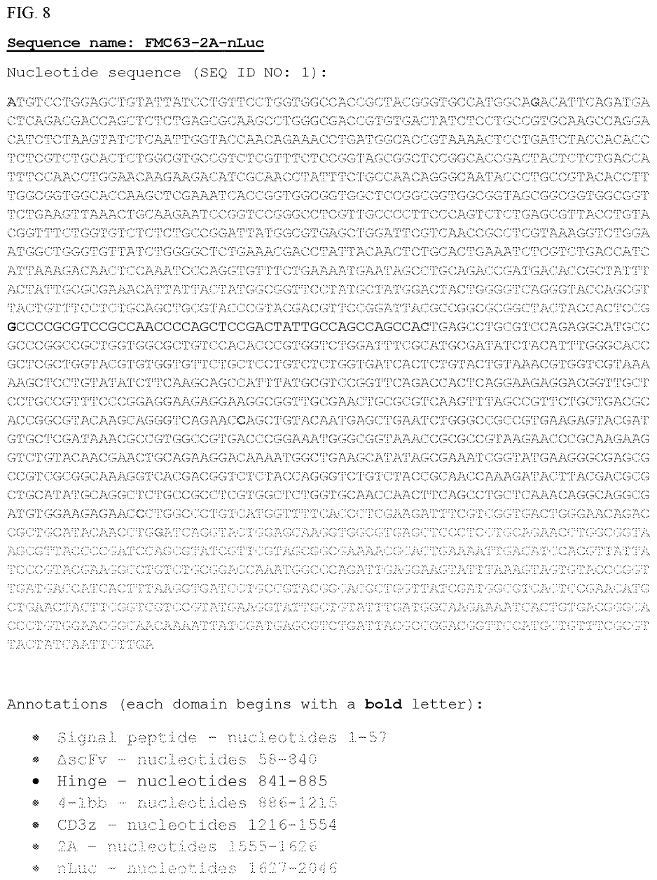

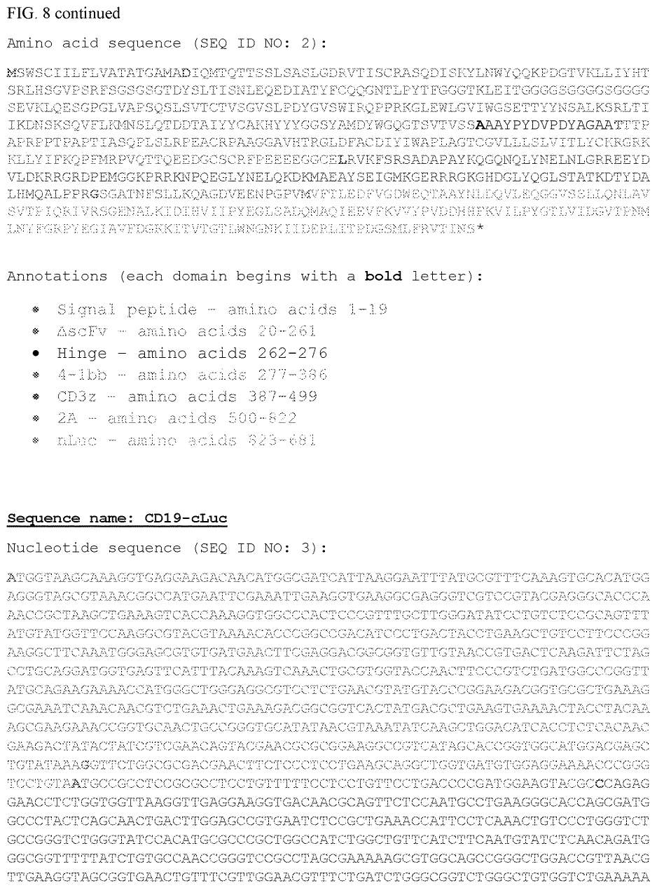

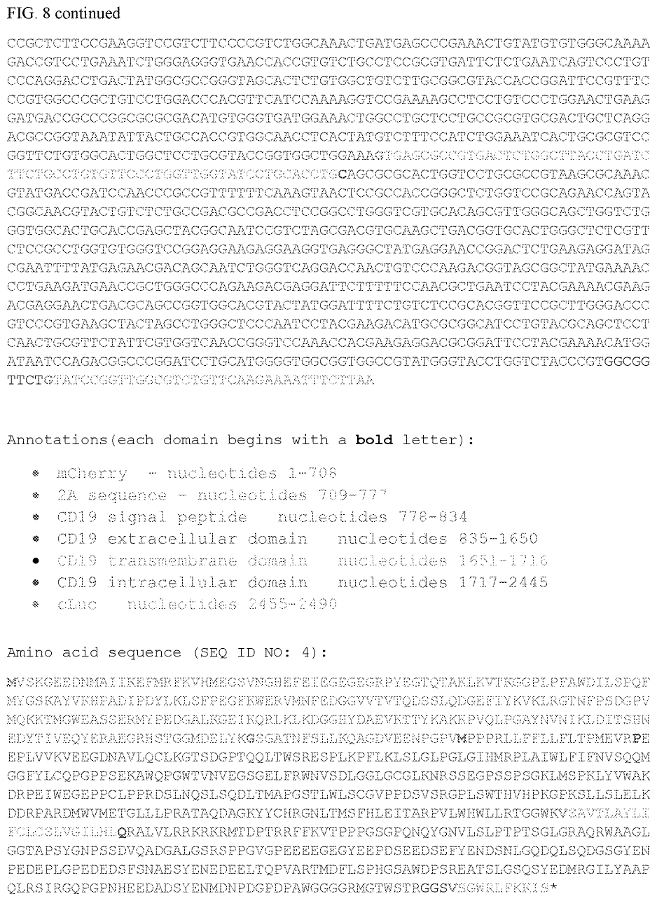

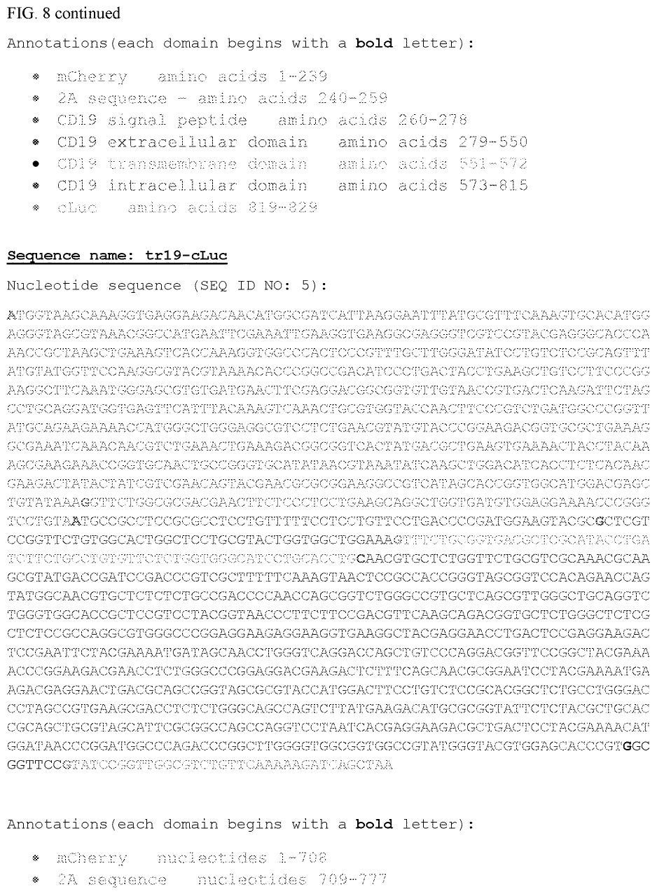

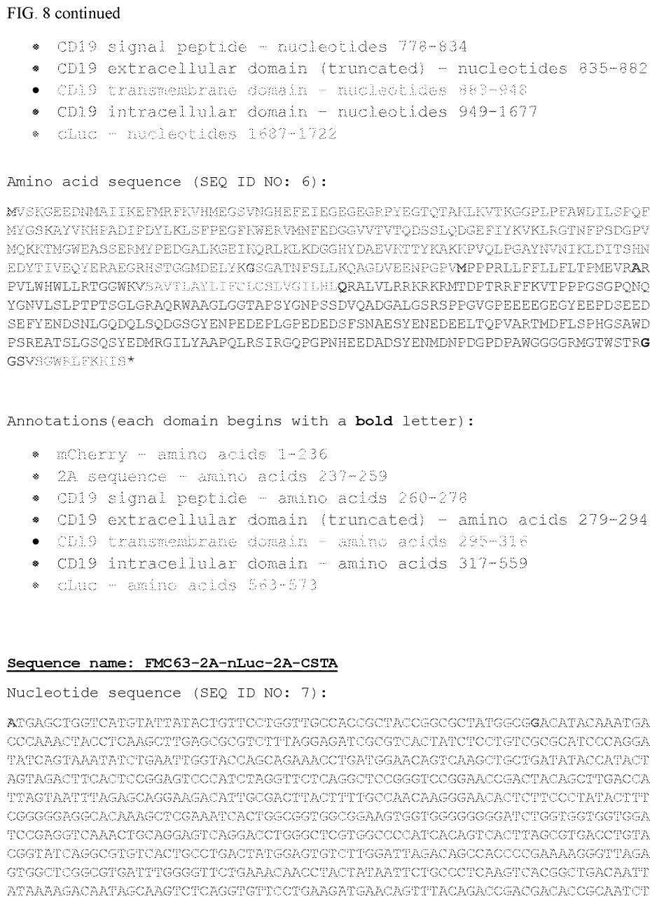

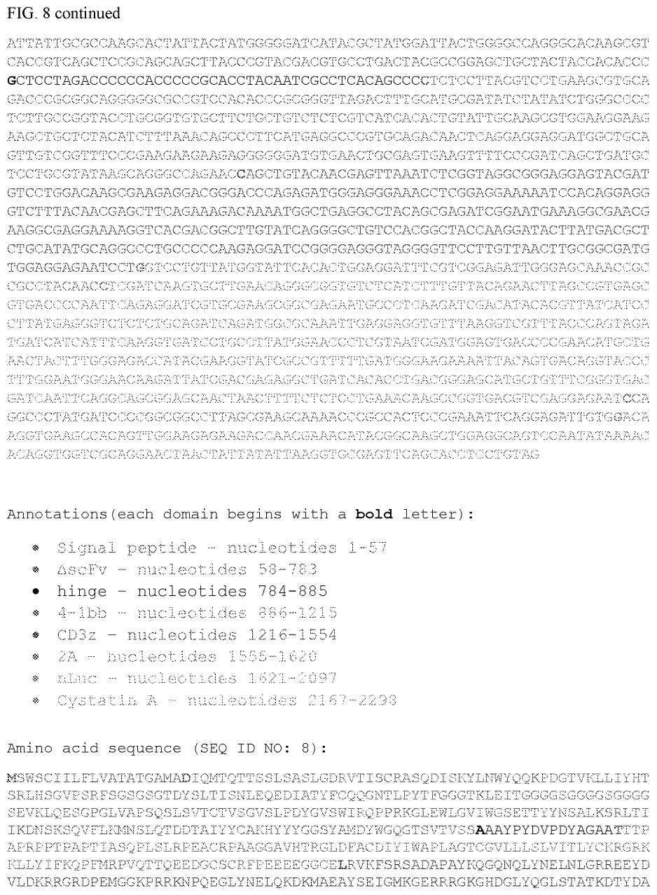

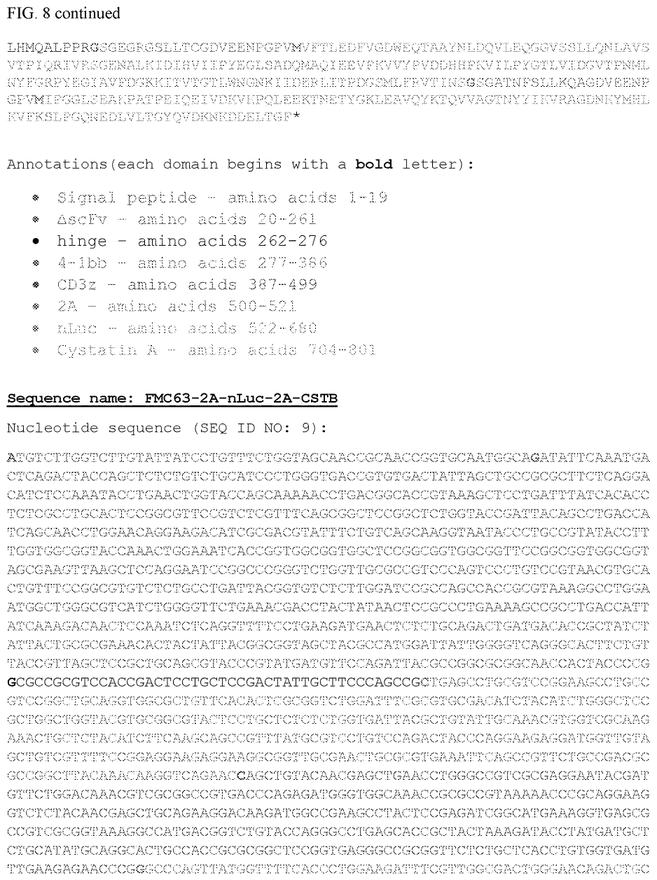

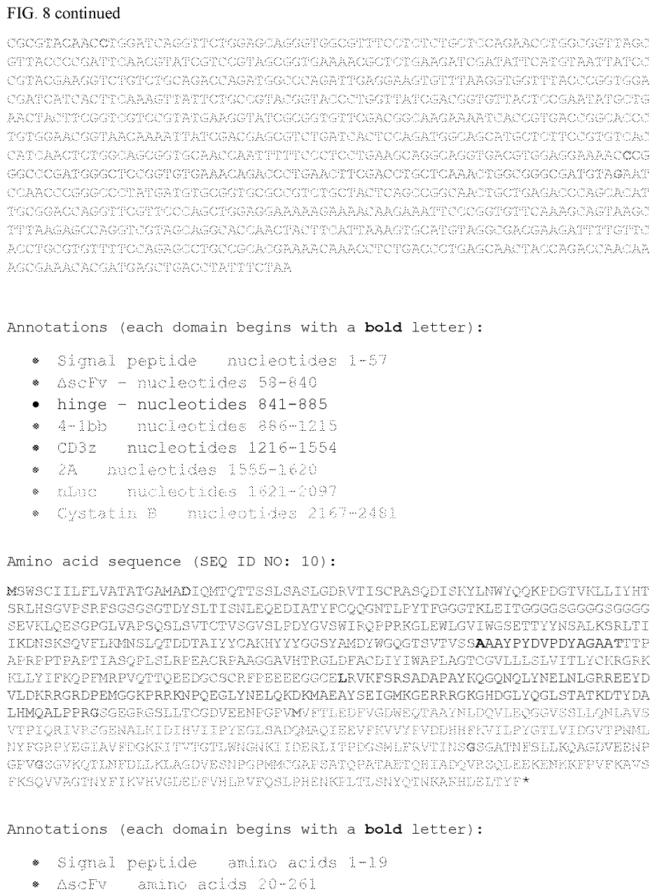

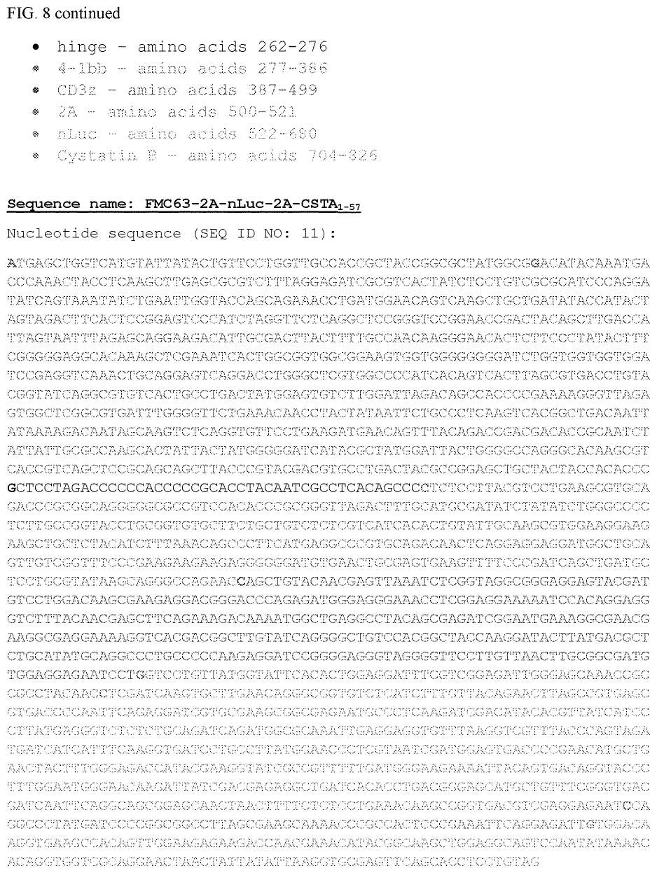

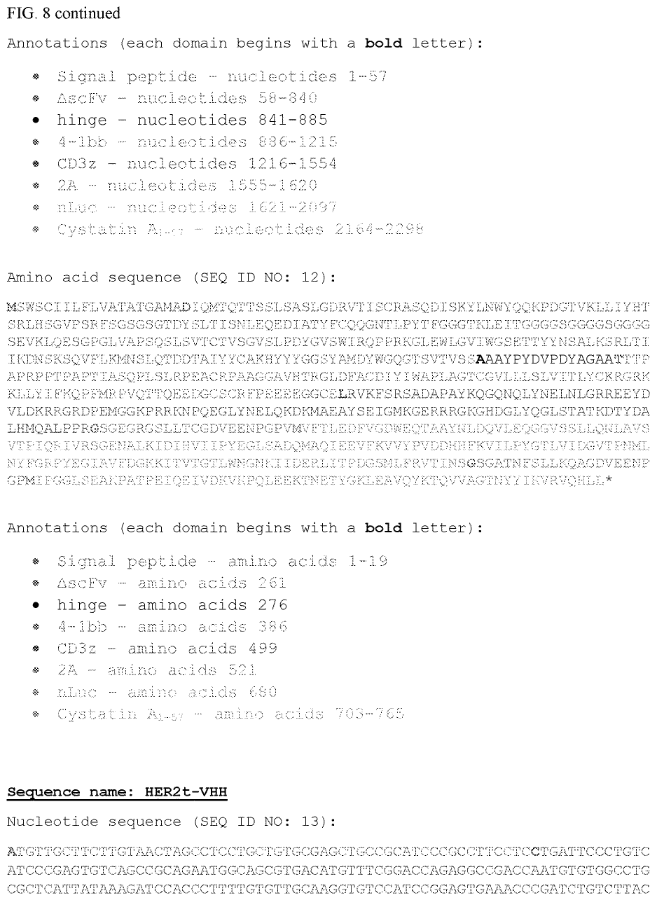

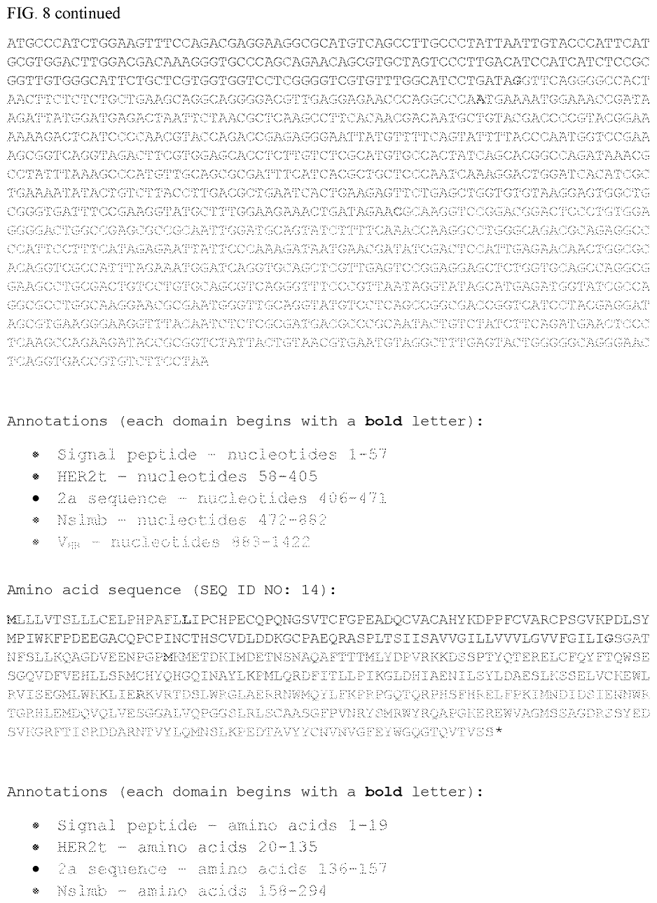

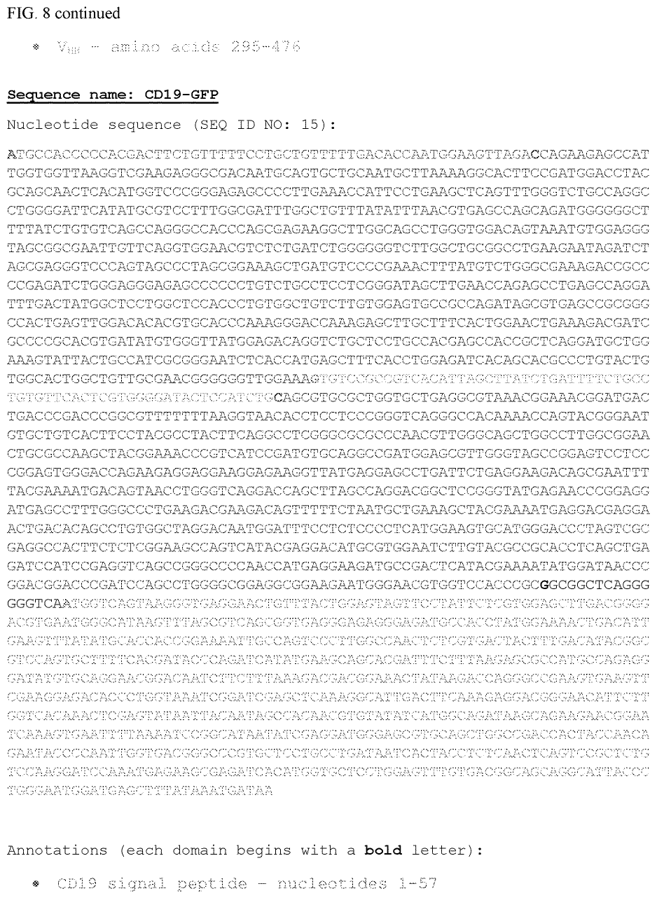

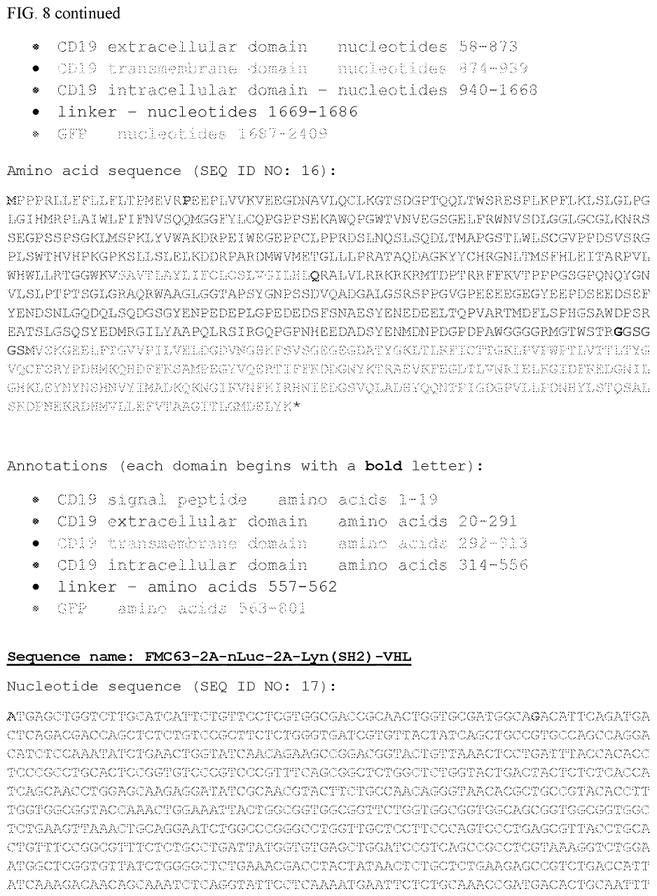

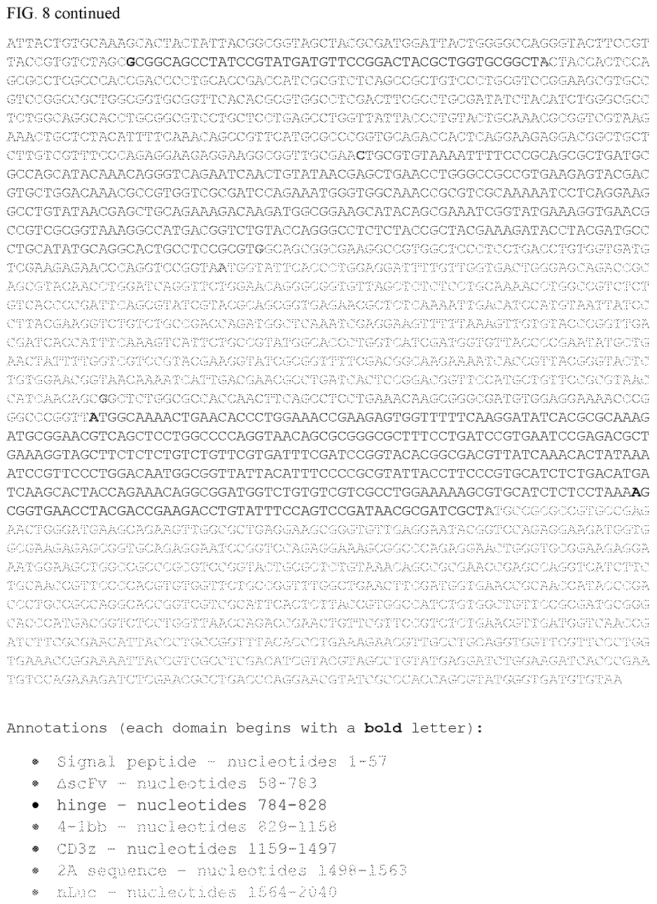

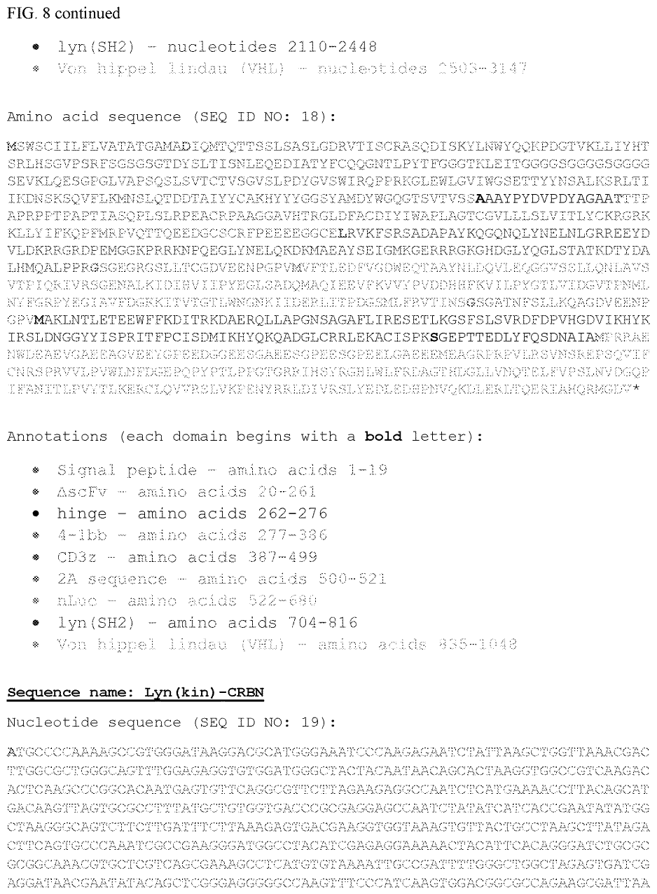

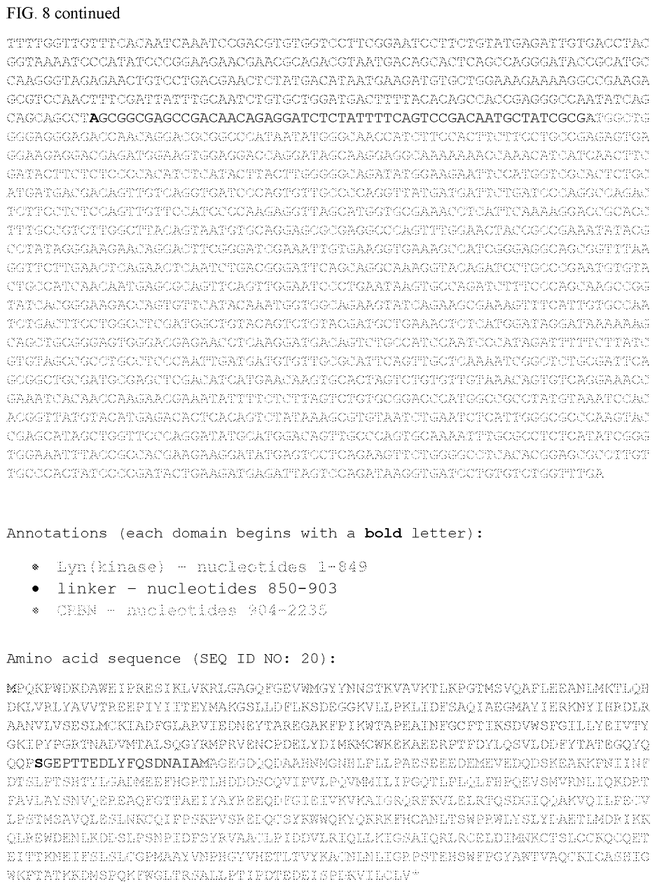

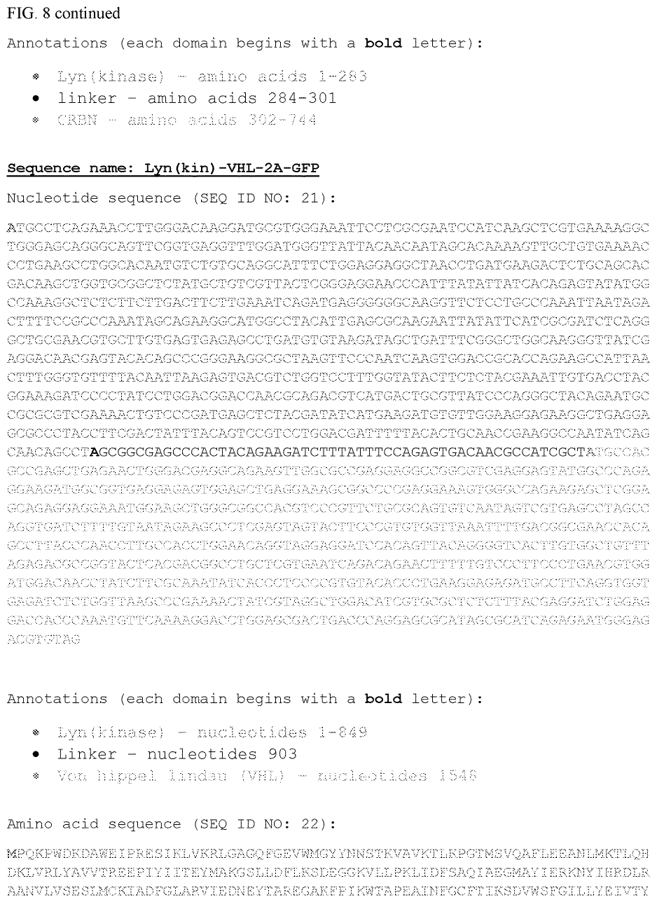

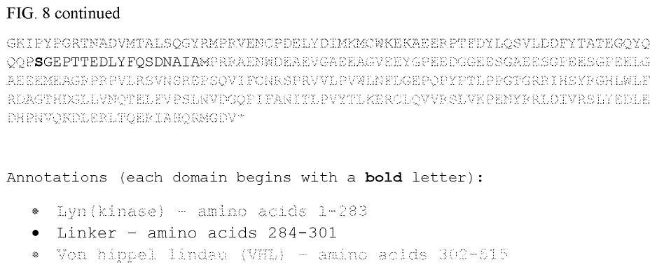

FIG. 7A shows the system used to examine the degradation of trogocytosed antigen. A CD19-GFP fusion protein (nucleic acid sequence—SEQ ID NO:15; amino acid sequence—SEQ ID NO:16) is transferred from the tumor cell (pink) to the CAR T cell (blue) via trogocytosis. Following trogocytosis, a GFP-targeting nanobody fused to Nslmb (TAD) binds to the CD19-GFP fusion protein, which is then polyubiquitylated. Polyubiquitylation targets the CD19-GFP fusion protein for proteasome-mediated degradation.

FIG. 7B shows the constructs used to assess CD19 degradation. Top: CAR T cell construct for the co-expression of nLuc and TAD (HER2t-VHH: nucleic acid sequence—SEQ ID NO:13; amino acid sequence—SEQ ID NO:14; bottom: tumor cell construct for the expression of a CD19-GFP fusion protein (nucleic acid sequence—SEQ ID NO:15; amino acid sequence—SEQ ID NO:16).

The results shown in FIG. 7C show the proof-of-concept used to demonstrate the effect of TAD expression on CD19-GFP. CD19-GFP-expressing 293T cells were transiently transfected with a plasmid encoding TAD using lipofectamine 2000 (Invitrogen #11668-019) according to manufacturer's instructions. 24 hours after transfection, cells were detached from wells of a plate using TrypLE express trypsin (Thermofisher #12605-010). Following trypsinization, cells were washed with 10 mL 293T media (DMEM [Thermofisher #11995073] supplemented with 10% fetal bovine serum and 1% penicillin/streptomycin). Cells were resuspended in 293T media to a concentration of 500,000 cells per mL. 100 μL of cells were plated per well. GFP fluorescence was measured using a Spark multi-mode plate reader (Tecan) to assess CD19-GFP expression. By transfecting 293T cells with TAD, a significant reduction in the amount of CD19-GFP expressed can be achieved, demonstrating that TAD can degrade CD19-GFP.

CD19-cLuc-expressing K562 cells were cocultured with CAR T cells±Cystatin overexpression±TAD at a 1:1 effector-target ratio for 30 minutes. Cells were prepared and the experiment was performed as described above. CD19 expression on CAR T cells was determined via flow cytometry. The results shown in FIG. 7D show that expression of TAD in CAR T cells results in significantly less GFP in CAR T cells, indicative of CD19-GFP degradation. Total CAR T cell counts were assessed via flow cytometry and normalized to ΔscFv. The results shown in FIG. 7E show that expression of TAD in CAR T cells results in significantly more CAR T cells at the end of the coculture, indicative of reduced fratricide.

Expression of CAR with or without TAD after staining with an anti-HA antibody recognizing an HA tag within the CAR, as measured via flow cytometry on Day 7 of manufacturing. Approximately 1×10⁶ CAR T cells with or without the expression of TAD were isolated, washed with FACS buffer, and centrifuged for 5 minutes at 400 xg. Cells were then stained with anti-hCD3, anti-HA, and a viability dye. Following staining, cells were washed with FACS buffer and centrifuged for 5 minutes at 400 xg. Cells were resuspended in 400 μL 1×PBS and acquired in a Beckton Dickinson LSRII flow cytometer, The results shown in FIG. 7F show that addition of TAD to the CAR T cell construct did not impact CAR T cell transduction efficiency.

Expansion of CAR T cells with or without TAD during manufacturing, as measured by automated cell counting using a Thermofisher Countess II. 10 μL of CAR T cells was diluted in 10 μL of Trypan Blue dye (Invitrogen #T10282). The results shown in FIG. 7G show that addition of TAD to the CAR T cell construct did not impact CAR T cell expansion.

Killing of Raji-luc cells by CAR T cells with or without the expression of Nslmb-VHH. CAR T cells and tumor cells were cocultured at the indicated effector-target ratios for 16 hours. Tumor survival was assessed by luminescence and described in the section entitled Titration of nLuc+ cLuc+ CAR T cells for the assessment of CompLuc sensitivity. The results shown in FIG. 7H show that addition of TAD to the CAR T cell construct does not impact CAR T cell cytotoxicity.

FIG. 7I shows the constructs used to assess a fully-human CD19 degradation system. Top: CAR T cell construct for the co-expression of CAR, nLuc, and lyn(SH2)-VHL fusion protein (FMC63-2A-nLuc-2A-Lyn(SH2)-VHL—nucleic acid sequence: SEQ ID NO:17, amino acid sequence: SEQ ID NO:18); middle: construct for the co-expression of a Lyn(kinase)-VHL fusion protein and GFP (Lyn(kin)-VHL-2A-GFP—nucleic acid sequence: SEQ ID NO:21, amino acid sequence: SEQ ID NO:22); bottom: construct for the co-expression of a Lyn(kinase)-CRBN fusion protein and GFP (Lyn(kin)-CRBN—nucleic acid sequence: SEQ ID NO:19, amino acid sequence: SEQ ID NO:20).

FIG. 7J left: CD19 degradation by TAD as measured by western blot using lysates from 293T cells expressing CD19 and transiently transfected with TAD or fully human degradation constructs. CD19-GFP-expressing 293T cells were transfected with plasmids encoding the indicated TAD constructs using lipofectamine 2000 (Invitrogen #11668-019) according to manufacturer's instructions. 24 hours after transfection, 1E7 cells were lysed using RIPA buffer (Thermofisher #89901) containing protease inhibitor (Roche #04693159001) for 30 minutes while rotating at 4° C. After 30 minutes, lysate was centrifuged at 14,000×g for 20 minutes at 4° C. After centrifugation, lysate was collected and concentration was determined using Pierce BCA assay (Thermofisher #23227) according to manufacturer's instructions. Samples were separated by SDS-poly-acrylamide gel electrophoresis, and separated proteins were trans-ferred to nitrocellulose membranes using an iBlot2 transfer system (Thermo Fisher Scientific). Membranes were blocked with 5% nonfat milk-tris-buffered saline and incubated with primary antibodies against CD19 or β-actin. Membranes were washed and developed using species-specific secondary anti-IgG/horseradish peroxidase antibodies and Western Lightning Plus-ECL solution (PerkinElmer). Bands were visualized and quantified on an iBright 1500 imaging system (Thermo Fisher Scientific). FIG. 7J right: quantification of bands normalized to beta-actin control. This data shows that expression of a fully human TAD construct composed of the SH2 domain of the protein Lyn and the ubiquitin E3 ligase VHL results in less CD19 via western blot.

FIG. 7K left: CD19 degradation by TAD as measured by western blot using lysates from 293T cells expressing CD19-GFP and transiently transfected with TAD or a fully human degradation construct using the Lyn(SH2) domain. Cells were transfected and lysed as described above. Samples were separated and analyzed via western blot as described above. Cells were lysed 24 hours after transfection. FIG. 7K right: quantification of bands normalized to beta-actin control. Expression of TAD results in significantly less CD19-GFP as measured by western blot.

FIG. 7L left: expression of Lyn(SH2)-VHL fusion protein as measured by western blot following production of CD19 CAR T cells transduced with the indicated constructs. CAR T cells were produced by gamma retrovirus transduction as previously described [44]. Cell lysate was generated, samples were separated, and western blot was performed as described in section

Primary antibodies against VHL β-actin were used. Cells were lysed following CAR T cell production. FIG. 7L right: quantification of bands normalized to beta-actin control. The data demonstrates the successful expression of fully-human TAD in CAR T cells, as measured by western blot.

### 5. Cathepsin B Causes Trogocytosis-Mediated CAR T Cell Dysfunction.

### 5A. Results

Chimeric antigen receptors (CAR) are genetically engineered proteins combining antigen binding domains with immune cell activation domains [45-47]. T cells and natural killer (NK) cells engineered to express CARs recognize tumor-associated antigens with high specificity and have been shown to be effective at targeting several hematologic malignancies [45,48-51]. It has previously been shown that CAR T cells and CAR NK cells rapidly transfer the targeted antigen from tumor cells to their own cell surface in a process called trogocytosis [52-54]. CAR-mediated trogocytosis (CMT) is associated with increased expression of apoptotic and exhaustion markers as well as reduced expansion [52-54]. In addition, it has been shown that T cells engineered to express the tumor antigen CD19 are efficiently killed by other CD19 CAR T cells (“fratricide”) [52]. Mechanistically linking CMT to exhaustion, fratricide, and reduced CAR T cell expansion has, so far, been elusive due to the lack of specific inhibitors of trogocytosis.

In the further studies discussed in the following paragraphs, an analytical framework was developed to investigate CMT and determine the effects of CMT on CAR T cell function as well as its mechanistic basis.

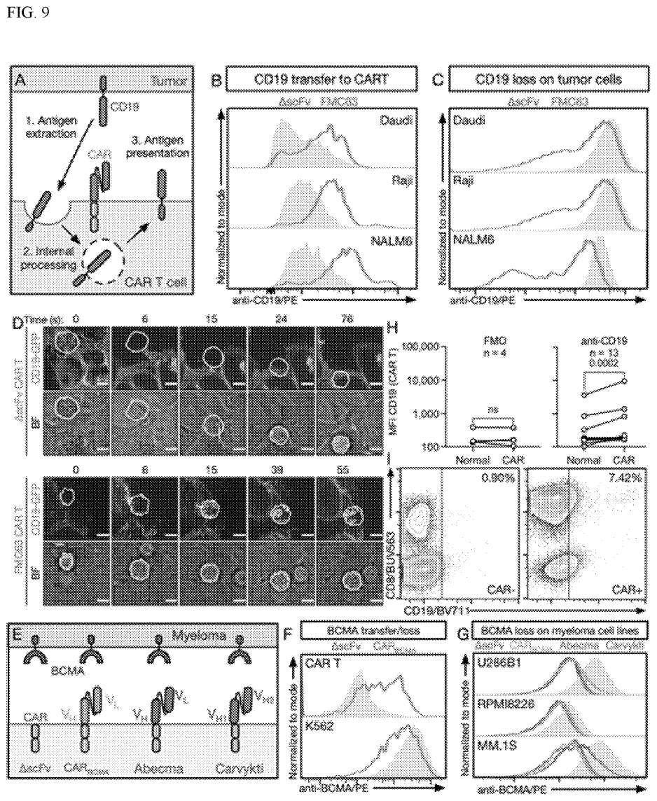

CAR T cells acquire target antigen via trogocytosis. CAR-mediated trogocytosis (CMT) is the extraction of the targeted tumor antigen from the tumor cell surface and its incorporation into the CAR T cell plasma membrane [55-55](FIG. 9A). Ex vivo, CMT occurs across cancer types [52,53] and target antigens, including CD19, CD22 [52], mesothelin [52], and BCMA [55]. Robust transfer of CD19 to CD19-targeting CAR T cells (clone: FMC63) was found as well as CD19 loss on tumor cells (FIG. 9B/C), which can be observed just seconds after CAR T cells contact target cells (FIG. 9D). Similarly, transfer of B cell maturation antigen (BCMA), the target of two clinically approved CAR T cell approaches for the treatment of multiple myeloma [48,49,56,57](FIG. 9E) to BCMA CAR T cells (FIG. 9F) and loss of BCMA from tumor cells (FIG. 9G) independent of CAR format (FIG. 9E) was confirmed. In the clinical setting, CMT had only been shown to occur in B cell lymphoma patients treated with CAR NK cells [54]. By analyzing peripheral blood mononuclear cells (PBMCs) from patients who had recently received CD19 CAR T cells, it was demonstrated that CMT also occurs in patients treated with CAR-expressing T cells (FIG. 9H/I).

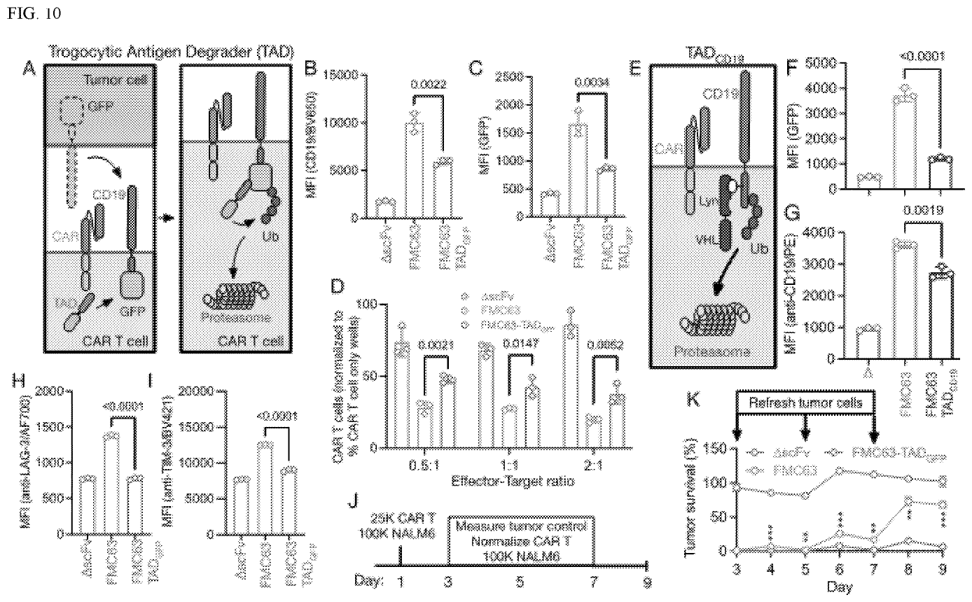

CAR-mediated trogocytosis directly causes CAR T cell dysfunction. The presence of trogocytosed antigen on CAR T cells is correlated with increased exhaustion and reduced viability [52-54] but it remains unknown if CMT is the cause of these effects. To answer this question, an approach was developed for the targeted degradation of trogocytosed CD19 fused to GFP (CD19-GFP) in CAR T cells (FIG. 10A). Expression of a trogocytosed antigen degrader (TAD), the E3-targeting domain Nslmb [58,59] fused to a GFP-binding protein (TAD_{GFP}), results in a significant reduction of total and surface CD19-GFP in 293T cells (FIG. 11A/B). Expression of TAD_{GFP} did not alter CAR T cell expansion during manufacturing or short-term antitumor activity (FIG. 11C/D).

When cocultured for 2 hours with K562 cells expressing CD19-GFP, conventional FMC63 CAR T cells showed increased levels of CD19 (FIG. 10B) and GFP (FIG. 10C) indicative of CMT. However, when co-expressing TAD_{GFP}, CD19 and GFP levels were significantly reduced in FMC63 CAR T cells after coculture. Targeted degradation of trogocytosed antigen was also validated using a fully human TAD (TAD_{CD19}) construct directly targeting CD19. TAD_{CD19} recognizes CD19 by using the Lyn kinase SH2 domain (FIG. 10E), which has been shown to bind the intracellular domain of CD19 with high affinity [60]. Similar to TAD_{GFP}-expressing CAR T cells, co-expression of TAD_{CD19} in FMC63 CAR T cells also resulted in reduced CMT (FIG. 10F/G). These data indicate that selective degradation of trogocytosed antigen is feasible in CAR T cells and that this approach can be used to explore the effects of CMT on CAR T cell function.

Using the TAD system, the effect of CMT on CAR T cell fratricide was investigated. It was found that TAD_{GFP} expression did not alter the proliferation of CAR T cells when co-cultured with CD19⁺ tumor cells (FIG. 11E). However, TAD_{GFP} expression resulted in significantly increased CAR T cell numbers after co-culture (FIG. 10D), indicating that CMT directly causes CAR T cell death, likely due to fratricide.

It has previously been shown that FMC63 can bind CD19 in cis [61] and could thereby cause persistent CAR signaling followed by T cell exhaustion (FIG. 11F). It was found that FMC63 CAR T cells that had acquired CD19 during co-culture with CD19-GFP-expressing K562 cells showed increased expression of exhaustion markers TIM-3 and LAG-3 compared to negative control T cells expressing a CAR without a binding domain (ΔscFv, FIG. 10H/I). Expression of TAD_{GFP} prevented TIM-3/LAG-3 upregulation on these cells demonstrating that increased exhaustion marker expression is the direct consequence of trogocytic antigen transfer to CAR T cells (FIG. 10H/I). CMT-induced fratricide and exhaustion could substantially limit long-term CAR T cell anti-tumor activity. In a serial coculture stress test of CAR T cell efficacy (FIG. 10J), it was found that TAD_{GFP} substantially increased the ability of CAR T cells to control B cell acute lymphoblastic leukemia (B-ALL, FIG. 10K).

These data demonstrate that CMT directly causes CAR T cell dysfunction and fratricide, and thereby limits CAR T cell expansion and anti-tumor activity.

Real-time detection of CAR-mediated trogocytosis by luciferase complementation assay. Little is known regarding the molecular and cellular drivers of CMT, and to date there are no high-throughput assays to systematically probe potential modulators of CMT as it occurs in real-time. To address this, a luciferase complementation assay was developed for the real-time detection of CMT (CompLuc, FIG. 12A). In the CompLuc assay, luciferase complementation occurs following the transfer of CD19 fused to the C-terminal fragment of NanoLuc [62](cLuc) to CAR T cells, which express the complementary cytosolic N-terminal NanoLuc (nLuc) fragment that has a high affinity for cLuc [62]. The CompLuc assay allows for highly sensitive detection of small numbers of nLuc⁺cLuc⁺ CAR T cells in cocultures (FIG. 12C). It was demonstrated that CMT, as quantified by CompLuc, is antigen- and CAR-dependent, and correlates with effector-target ratio (FIG. 12D-F) and flow cytometry endpoint analysis (FIG. 12D/F).

To validate the CompLuc assay, the effect of an established modulator of CMT, the actin polymerization inhibitor cytochalasin D [52,63], was determined. Treatment of CAR T cells with cytochalasin D significantly reduced CMT in a dose-dependent manner as measured by CompLuc assay (FIG. 10G), demonstrating the ability of the assay to identify modulators of CMT.

Extracellular cathepsin B is a key driver of CAR-mediated trogocytosis. CMT may be the result of incomplete target cell killing followed by the endocytosis of target cell fragments, as suggested by the simultaneous effect of cytochalasin D on CMT and CAR-mediated cytotoxicity (FIG. 12H). However, it was hypothesized that, while the molecular mechanisms driving target cell killing and trogocytosis may overlap, CMT could in part be mechanistically distinct from cytotoxic function.

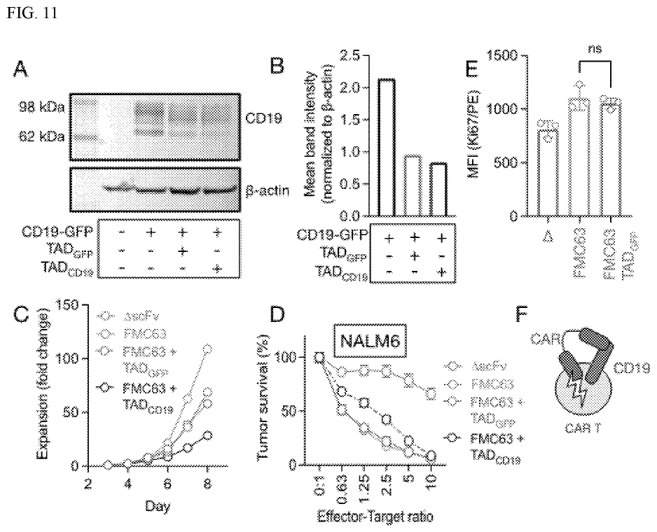

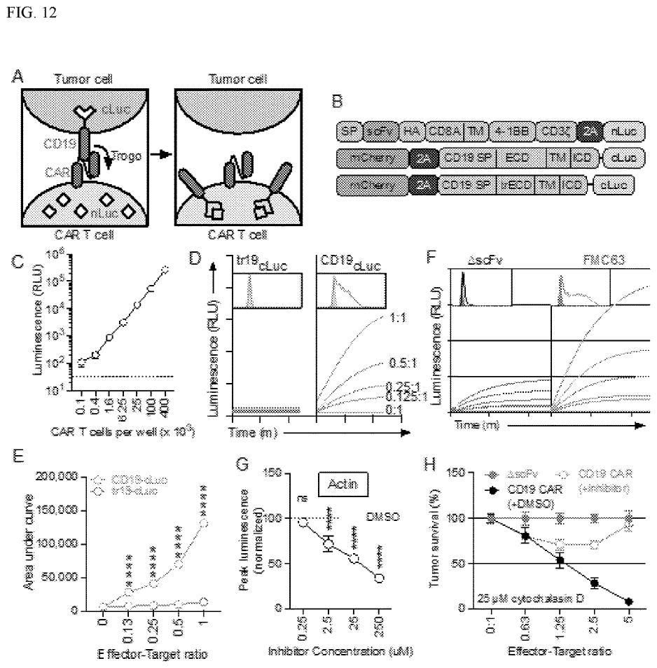

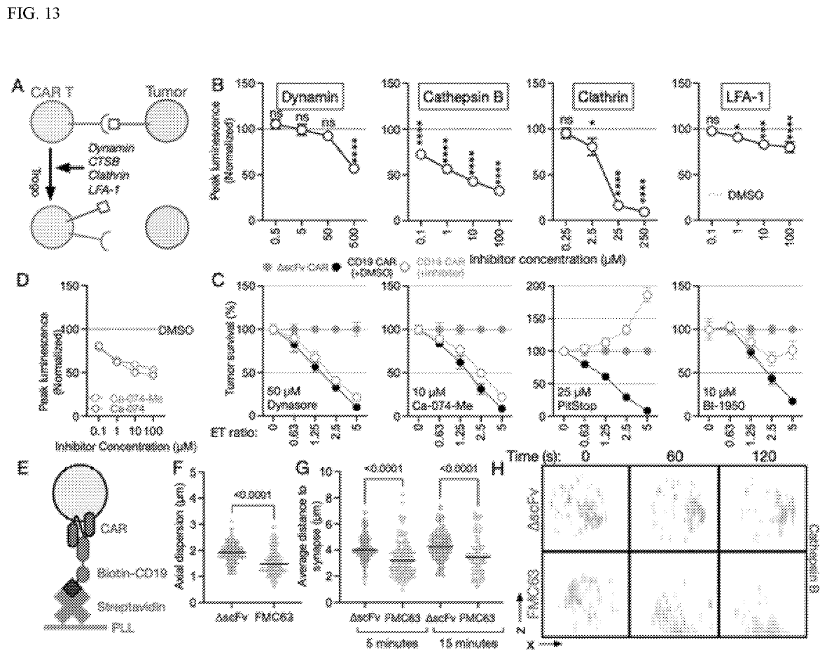

To identify functional systems that could be specifically involved in CMT but not cytotoxicity, the effect of inhibiting various processes at the T cell immune synapse on CMT [52,63-66] was explored (FIG. 13A). It was found that inhibition of cathepsin B (CTSB) with the small molecule inhibitor Ca-074-Me in FMC63 CAR T cells reduced CMT in a dose-dependent manner as measured by CompLuc (FIG. 13B) without substantially altering CAR T cell cytotoxicity (FIG. 13C). Using a membrane-impermeable inhibitor of CTSB, it was found that inhibition of extracellular CTSB alone is sufficient to prevent CMT (FIG. 13D).

CTSB is a ubiquitously expressed cysteine protease, primarily localized to lysosomal and endosomal compartments under physiological conditions, where it is primarily involved in protein degradation and turnover [67,68]. However, CTSB is also found in the cytosol and exocytic vesicles, retains its catalytic activity at neutral pH [69], and has been shown to line exocytic granules of cytotoxic T lymphocytes [70]. In addition to proteolytic degradation within the endosome and lysosome, CTSB has been implicated in the degradation and remodeling of components of the extracellular matrix, such as collagen and fibronectin, thereby promoting the invasion and metastasis of tumor cells [69,71,72]. Similarly, CTSB may contribute to the extraction of antigen-rich membrane fragments of target cells prior to their transfer to CAR T cells.

To further support the potential role of extracellular CTSB produced by CAR T cells in CMT, it was determined whether CTSB in CAR T cells is actively transported to the immune synapse upon antigen contact. To this end, FMC63 CAR T cells expressing CTSB fused to mCherry were generated and these cells were exposed to immobilized CD19 (FIG. 13E). It was found that CTSB in FMC63 CAR T cells but not in ΔscFv CAR T cells, rapidly localized to the immune synapse following CAR T cell antigen recognition (FIG. 13F-H), correlating with increased CMT as measured by CompLuc and flow cytometry (FIG. 12F). These findings indicate that transport of CTSB to the immune synapse and its subsequent secretion by CAR T cells are necessary for CMT.

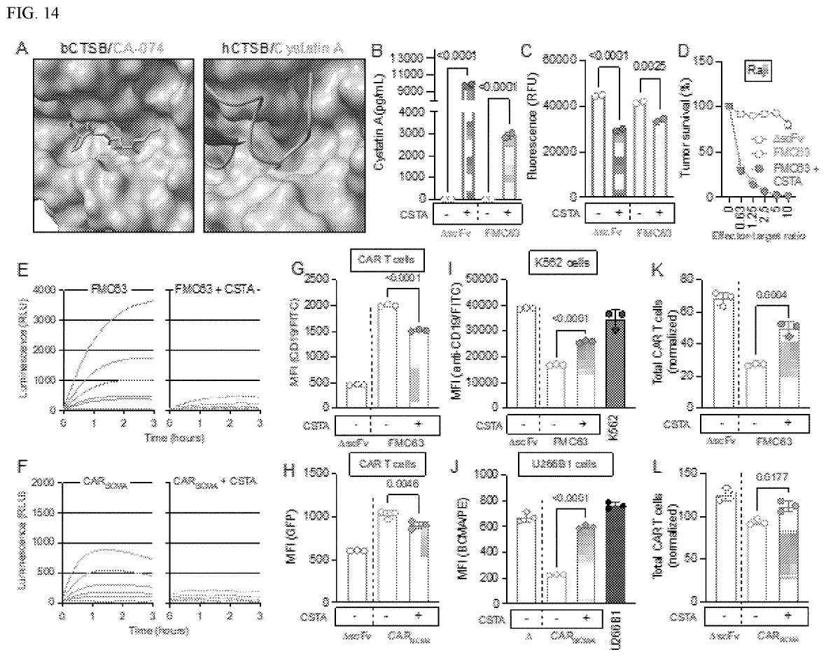

Cystatin abundance controls CTSB activity and CMT. CTSB is ubiquitously expressed across tissues and cell types and its activity is regulated through a network of endogenous protease inhibitors called cystatins [73]. Cystatin A (CSTA) has been described as the main regulator of CTSB activity [74] and, similar to Ca-074, CSTA inhibits CTSB through insertion of a hydrophobic wedge into the active-site cleft of CTSB [75](FIG. 14A). To determine if CSTA abundance directly regulates CMT in CAR T cells, CAR T cells were engineered to stably overexpress CSTA (CAR_{CSTA}). It was confirmed that CSTA overexpression led to significantly increased amounts of CSTA protein in CAR T cells (FIG. 14B), which resulted in overall reduced CTSB activity (FIG. 14C) without altering in vitro expansion or CAR T cell phenotype (data not shown). When co-cultured with tumor cells expressing the respective target antigens, CD19 CAR_{CSTA} cells showed equivalent anti-tumor activity compared to conventional CAR T cells (FIG. 14D) whereas BCMA CAR_{CSTA} cells showed slightly improved short-term antitumor activity (data not shown). When co-cultured with CD19⁺ or BCMA⁺ tumor cells, significantly reduced CMT in CAR_{CSTA} cells was observed as measured by CompLuc (FIG. 14E/F) despite comparable CAR T cell activation (data not shown). CMT was similarly reduced when overexpressing the closely related protein cystatin B [76] or truncated CSTA₁₋₅₇ containing the hydrophobic wedge (data not shown). Overexpression of cystatins reduced both antigen transfer to CAR T cells (FIG. 14G/H) and antigen loss on tumor cells (FIG. 14I/J), suggesting inhibition of CMT at the antigen extraction step. This CSTA-mediated inhibition of CMT resulted in significantly increased CD19 and BCMA CAR T cell expansion when exposed to tumor cells in vitro (FIG. 14K/L) and in vivo (data not shown), further demonstrating that inhibition of CMT improves CAR T cell persistence.

Taken together, these data demonstrate that CTSB expressed in CAR T cells is rapidly transported to the immune synapse upon antigen stimulation and, following secretion, confers extraction of target antigen from tumor cells and subsequent transfer to CAR T cells. The abundance of the endogenous CTSB inhibitor CSTA in CAR T cells efficiently controls CMT, with high CSTA abundance significantly prolonging in vitro and in vivo CAR T cell expansion/persistence.

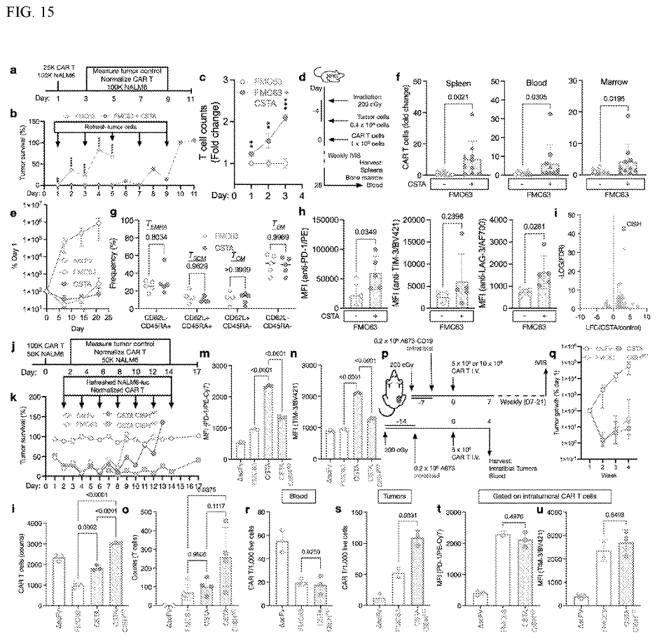

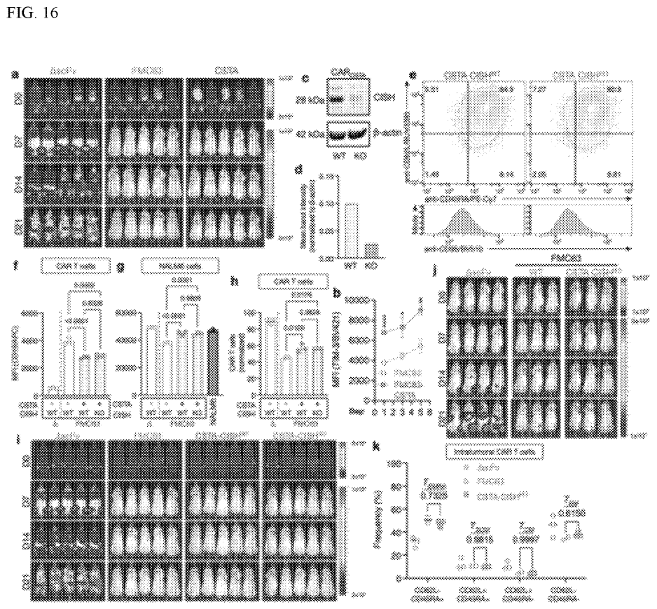

Cystatin allows long-term CAR T-cell persistence and antitumor activity. It was hypothesized that the CSTA effect on CAR T-cell survival in short-term cocultures could also lead to increased long-term CAR T-cell persistence and antitumor activity of CAR_{CSTA} T cells. Therefore, a serial cytotoxicity assay was performed to determine CAR_{CSTA} T-cell antitumor activity over an extended period of time (FIG. 15a). It was found that CAR_{CSTA} T cells significantly prolonged tumor control (FIG. 15b) and increased CAR T-cell numbers was observed over time (FIG. 15c). In an in vivo B-ALL tumor model (FIG. 15d), tumor control was observed in all groups treated with CD19-targeting CAR T cells (FIG. 15e, FIG. 16a) and again significantly increased CAR_{CSTA} T-cell numbers were found across tissues (FIG. 15f). When analyzing the phenotype of these persisting CAR T cells, it was found that CSTA overexpression did not significantly alter the T-cell subset composition (FIG. 15g).

CISH upregulation associated with CSTA overexpression. Next, the expression of exhaustion markers in CAR T cells from in vitro serial cytotoxicity and in vivo experiments was explored. It was found that despite their substantially increased persistence in both experiments, CAR_{CSTA} cells showed significantly increased levels of exhaustion (FIG. 15h, FIG. 16b). To explore potential causes of this increased exhaustion, bulk RNA sequencing of FMC63 CAR and CAR_{CSTA} T cells was performed at the end of production. It was found that CSTA overexpression resulted in the spontaneous upregulation of cytokine-inducible SH2-containing protein (CISH), which has previously been implicated as a key driver of CAR T-cell exhaustion (FIG. 15i). To determine whether this upregulation of CISH could have caused the increased exhaustion of CAR_{CSTA} T cells, an efficient CRISPR/Cas9-mediated CISH knockout was established (FIG. 16c/d). CISH^{KO} did not result in changes in T-cell subset composition at the end of production (FIG. 16e) and did not affect the inhibition of CMT by CSTA overexpression (FIG. 16f-h), indicating that the effect of CSTA on CMT is not mediated by CISH.

CISH^{KO} prevents CSTA-mediated exhaustion. Next, a serial cytotoxicity assay was performed using CAR_{CSTA} cells with and without CISH^{KO} (FIG. 15j) and found that CISH^{KO} significantly prolonged tumor control and further increased CAR T-cell persistence over time (FIG. 15k/l). In addition, CISH^{KO} almost entirely prevented exhaustion in CSTA-expressing CAR T cells in vitro (FIG. 15m/n), indicating that CSTA-induced upregulation of CISH caused increased levels of exhaustion in CAR_{CSTA} cells. This finding was validated in the same in vivo model described above and again observed complete tumor control and increased persistence in CAR_{CSTA} CISH^{KO} CAR T cells (FIG. 15o, FIG. 16i).

It was hypothesized that CMT could be more pronounced in the solid tumor setting due to the higher density of tumor cells leading to more extensive antigen transfer and fratricide. Therefore, a solid tumor in vivo model was established by intratibial implantation of A673 Ewing sarcoma cells engineered to express CD19 (FIG. 15p). This model was characterized by rapid localized tumor growth and comparable tumor control by systemically injected wild-type FMC63 CAR T cells as well as CAR_{CSTA} CISH^{KO} CAR T cells (FIG. 15q, FIG. 16j). In this model, it was found that CSTA expression did not alter CAR T-cell numbers in the peripheral blood (FIG. 15r) but significantly increased intratumoral CAR_{CSTA} T-cell numbers, indicating that CSTA overexpression does not negatively affect tumor infiltration by CAR T cells (FIG. 15s) and instead efficiently prevents intratumoral CAR T-cell fratricide. The exhaustion and phenotype of CAR_{CSTA} CISH^{KO} cells were comparable to those of FMC63 CAR T cells, indicating that CISH^{KO} robustly prevents CSTA-mediated exhaustion in solid and hematologic settings (FIG. 15t/u, FIG. 16k).

Taken together, these data indicate that CSTA overexpression leads to significantly reduced CMT, as well as increased long-term CAR T-cell persistence and in vitro tumor control. CSTA-mediated induction of exhaustion via CISH could be overcome by simultaneous CISH^{KO}, further enhancing functional CAR T-cell persistence.

### 5B. Materials and Methods

Study approval. All recombinant DNA and biosafety work was approved by the institutional biosafety committees at University of Maryland, Baltimore (protocol IBC-6040). Animal experiments were approved by the institutional animal care and use committee at the University of Utah (protocol 18-1104). Patient samples were collected under the Institutional Review Board (IRB)-approved protocol 2043GCCC (IRB H0091736, PI D. Atanackovic) after obtaining informed consent.

Cell lines and primary human cells. Raji, NALM6, Daudi, DB, Toledo, MM.1S, RPMI8226, U266B1, K562, A673, TC-71, and Phoenix-AMPHO cells were purchased from the American Type Culture Collection (ATCC) and cultured according to ATCC instructions. Lenti-X 293T cells were purchased from Takara and cultured according to the manufacturer's instructions. Cell lines were authenticated by their respective supplier. Healthy donor buffy coats were obtained from the New York Blood Center. PBMCs from healthy donors were isolated from buffy coats by density gradient using FillPaque (GE) as previously described [57,78]. Primary human T cells were cultured in AIM-V medium (Invitrogen 12055-083) supplemented with 5% Human serum (Sigma H4522-100 ML), 1% Pen/Strep (Thermofisher 15140-122) (T cell media), and 40 IU IL-2 (R&D Systems #202-IL-10). All cells were cultured at 37° C., 5% CO₂.

Vector constructs. All vectors generated for this study were produced by Twist Biosciences. FMC63-nLuc contains the CD19-specific scFv fragment FMC63 [79]. All CAR constructs contain the CD8a hinge/transmembrane domain, 4-1BB costimulatory domain, and CD3 z domain. CAR constructs were generated using existing binders for CD19 (clone: FMC63 [80] or CAT [81], FolRα (clone: C4) [82], BCMA (Carvykti and Abecma) [83], and LINGO1 (clone: Li82) [84]. For some constructs, the full-length sequence of human cystatin A (UniProt, P01040) was synthesized and cloned downstream of the respective CAR and nLuc fragment separated by a P2A sequence (Twist Bioscience). DNA was isolated using Endofree Plasmid Maxi Kit (Qiagen 12362) following the manufacturer's protocol. Plasmid concentration was measured using a NanoDrop One instrument (Thermo). All DNA constructs were stored at −20° C.

Clinical data. Whole blood was drawn from patients receiving CAR T cell therapy 7-28 days after CAR T cell injection. Peripheral blood mononuclear cells (PBMCs) were isolated by density gradient using Ficoll Paque as previously described [57,78]. PBMCs were stained with anti-hCD3, anti-hCD4, anti-hCD8, anti-CAR (Miltenyi #130-127-342), and anti-hCD19, anti-hCD27, anti-hCD137, and 7-AAD and analyzed by flow cytometry. Samples were collected under Institutional Review Board (IRB)-approved protocol 2043GCCC (IRB H0091736, PI D. Atanackovic).

Gamma retrovirus Production. Gamma retrovirus was produced using Phoenix-AMPHO cells (ATCC, catalog no. CRL-3213). Phoenix-AMPHO cells were transiently transfected with 16 μg of plasmid DNA using Opti-MEM Reduced Serum Medium (Thermofisher, catalog no. 31985070) and lipofectamine 2000 (Invitrogen 11668-019) according to manufacturer's instructions. During transfection, cells were cultured in Dulbecco's Modified Eagle Medium (DMEM, Thermofisher 11995073) supplemented with 10% FBS. Virus-containing supernatant was filtered using STERIFLIP™ Sterile Disposable Vacuum Filter Units (Millipore Sigma SEIM003M00). Virus was concentrated using Retro-X Concentrator (Takara, 631456). The following day, concentrated virus was centrifuged at 1500×g for 45 minutes at 4° C. Supernatant was removed and concentrated virus was resuspended in 1.5 mL complete T cell media.

Transgenic T cell production and expansion. CAR T cells were generated as previously described [55,77,80]. Buffy coats from healthy donors were obtained from the New York Blood Center and peripheral blood mononuclear cells (PBMCs) were isolated using Ficoll-Paque and cryopreserved until use. PBMCs were thawed and cultured overnight in complete T cell media. PBMCs were stimulated for 2 days with anti-CD3/anti-CD28 T cell activation beads (Thermo, catalog no. 11131D) in the presence of interleukin-2 (IL-2; 40 IU/ml; R&D Systems, catalog no. 202-IL-010) in complete T cell media and incubated at 37° C., 5% CO₂. Bead-stimulated cells were transferred to RetroNectin-coated (Takara) virus-containing plates and incubated overnight. Transduction was repeated the next day before counting and diluting cells to 0.4×10⁶ cells/ml. After the second transduction, cells were grown for an additional 7 days before removing beads using a DynaMag-15 magnet (Thermo Fisher Scientific). IL-2 was replenished every 2 days to 40 IU/ml. Cells were frozen in 90% fetal bovine serum/10% dimethyl sulfoxide and stored in liquid nitrogen. CAR T cell transduction efficiency and phenotype were determined by flow cytometry. CAR T cells were washed with FACS buffer and stained with antibodies targeting hCD3, HA, hCD4, hCD8, hCD95, hCD62L, and hCD45RA. Because BCMA CAR_{CSTA} T cells showed substantially lower transduction efficiency than conventional BCMA CAR T cells, after production, both products were FACS sorted following anti-HA (CAR) staining using a FACSAria cell sorter (BD) prior to subsequent functional assays.

To generate CISH^{KO} CAR T cells, on day 5, anti-CD3/CD28 T activator beads (Thermo Fisher) were removed from the transduced cells by magnetic separation. CRISPR ribonucleoprotein (RNP) particles containing 25 μg Cas9 only (mock/WT) or Cas9 and 150 μmol synthetic TrueGuide gRNA (CISH target sequence: 5′-GACAGCGUGAACAGGUAGCU-3′; SEQ ID NO: 23) were prepared according to the manufacturer's instructions (Thermo). A total of 3×10⁶ cells were electroporated in the presence of CRISPR-RNPs using the NEON electroporation system (Thermo) using the following settings: 1,600 V, 10 ms, 3 pulses. Following electroporation, the cells were transferred to prewarmed media and incubated for 2 hours at 37° C. Following incubation, new anti-CD3/CD28 T-cell activation beads were added to the electroporated cells at a ratio of 3:1, and the cells were grown for an additional 6 days before removing the beads using a DynaMag-15 magnet (Thermo Fisher). IL-2 was replenished every 2 days to 40 IU/ml. Cells were frozen in 90% FCS/10% DMSO and stored in liquid nitrogen.

Imaging Substrate Preparation. Eight-well chambers (Cellvis, catalog no. C8-1.5H-N) were used for all experiments. For CAR T cell activation on CD19-coated surfaces, 8-well chambers were coated with 0.01% poly-L-lysine (PLL) solution diluted in distilled water for 10 minutes at room temperature. PLL was aspirated from each well, and the chambers were allowed to air-dry for 1 hour at 37° C. PLL-coated dishes were then incubated overnight at 4° C. with a 10 μg/mL solution of NeutrAvidin (Thermo Scientific, catalog no. 31000) in 1× Dulbecco's phosphate-buffered saline (DPBS). After overnight incubation, coated wells were washed with 1×DPBS at room temperature and incubated at 37° C. with a 10 g/mL solution of biotinylated human CD19 protein (ACRO Biosystems catalog no. CD9-H82E9) in DPBS for 2 hours at 37° C. Prior to the experiment, coated wells were washed three times with RPMI 1640 phenol red-free imaging medium. For coculture experiments, 8-well chambers wells were incubated with 10 μg/mL fibronectin (MilliporeSigma, catalog no. 34-163-11MG) in DPBS at room temperature for 1 hour prior to seeding with 293T cells stably expressing CD19-GFP. 293T cells were seeded at a concentration of 5×10⁵ cells per well in complete growth media, followed by an overnight incubation at 37° C., 5% CO₂. Prior to imaging, wells were washed three times with warm complete imaging media, consisting of a 1:1 ratio of RPMI 1640 supplemented with 5% fetal bovine serum (FBS) and DMEM supplemented with 10% FBS. The washing process was performed thrice to ensure removal of any residual media while leaving a known volume in the wells.

ConfocalMicroscopy. Confocal microscopy was conducted using an inverted microscope (Nikon Ti-E PFS, Nikon Inc.) equipped with a 100× Silicone objective lens. Imaging was performed with a Prime BSI camera (Photometrics). Image acquisition protocols were managed using Nikon Elements software, and images were cropped in FIJI for further analysis. All live cell imaging was done with imaging chambers placed in a stage-top Okolab Incubator (Okolab S. R. L.) pre-equilibrated to 37° C. with 5% CO₂. For live cell imaging on glass, activated CAR T cells suspended in RPMI 1640 medium supplemented with 5% FBS were deposited onto CD19-coated surfaces. Imaging was started between 3 to 6 minutes after CAR T cells expressing CTSB-mCherry were added to a biotin-CD19-coated coverslip. For each well, timelapse images of a 3D volume (planes with z spacing of 0.3 m to span the cell from the top to the bottom) were acquired every 3 minutes for 60 minutes. For coculture experiments, imaging was started between 3 to 6 minutes after CAR T cells expressing CTSB-mCherry were dropped onto a layer of 293T cells expressing CD19-GFP seeded on a coverslip, at a concentration of 7×10⁴ cells per drop. Brightfield imaging was used to identify cells attached to the apical surface of HEK293 cells. The synaptic plane between a CAR-T cell and a HEK293 cell was identified and designated as the home plane for acquisition of Z-stack time-lapse movies. Two-channel images using 488 nm and 561 nm lasers (for GFP and mCherry imaging respectively) were acquired every 3 minutes for 1 hour and 15 minutes utilizing a Z-spacing of 0.3 or 0.6 m, with brightfield images taken at the home plane.

Image analysis was carried out in MATLAB (Mathworks, Inc.) using custom scripts. The plane of the synapse was determined using the actin channel. After background subtraction, axial intensity gradients are estimated for every voxel of sufficient intensity within the ROI. Below the cell, these gradients are typically positive due to the Airy pattern of the PSF. The synapse is taken as the first plane for which the gradients are no longer consistently positive.

Estimating the average distance of CTSB to the synapse. The center of fluorescence (COF) of CTSB is defined similarly to the center of mass. The voxel positions are weighted by CTSB intensity after background subtraction and thresholding to obtain the COF. The average CTSB distance is then calculated from the axial (z) distance of the Cathepsin B COF to the plane of the synapse.

Characterizing CTSB axial dispersion. The axial dispersion is an estimate of the average distance of CTSB molecules to the COF. The axial (z) distance of each voxel to the COF is determined, and the axial dispersion is defined as the average of these distances weighted by CTSB intensity after background subtraction and thresholding. Voxels with sufficiently low signal do not contribute to the calculation due to the thresholding procedure.

CTSB clustering at the synapse. The pair auto-correlation function g(r) of CTSB is computed at the synapse using the actin channel as a mask. The actin channel is segmented by smoothing with a Gaussian filter, generating an initial mask by applying k-means clustering (k=2) after a log transformation, and then applying morphological operations to connect and smooth the initial mask. The clustering coefficient, gₐᵥₑ, is computed by averaging over all radial bins with 0.25 m or 0.5 m.

CompLuc-based trogocytosis assay. Transduced, cryopreserved nLuc+ CAR T cells were thawed and cultured in complete T cell media supplemented with 40 IU IL-2 for 48 hours prior to use. nLuc-expressing CAR T cells were cocultured with CD19-cLuc-expressing K562 tumor cells at the specified effector-target ratios. CAR T cells and tumor cells were resuspended in Opti-MEM reduced serum media. Live Cell Substrate (Promega, catalog no. N2011) was prepared according to manufacturer's instructions. K562 tumor cells and prepared Live Cell Substrate were added to wells of a black 96-well plate and luminescence was measured to assess baseline luminescence. CAR T cells were added to appropriate wells and luminescence was measured every minute for three hours at 37° C. Luminescence was measured using a Spark multi-mode plate reader (Tecan).

Flow cytometry-based trogocytosis assay. A flow-cytometry based trogocytosis assay was used to confirm results observed in CompLuc, to assess CD19 or BCMA levels on CAR T cells and tumor cells, and to quantify CAR T cells. 5×10⁴ target cells were seeded in wells of a 96-well round bottom plate. Various ratios of CAR T cells produced from one of three healthy donors were cocultured with target cells for 1 hour at 37° C., 5% CO₂. Following coculture, cells were resuspended by gentle pipetting and transferred to wells of a 96-well V bottom plate for washing and staining. Cells were stained with Zombie violet or Zombie NIR fixable viability dye, and antibodies for hCD3, HA, hCD19 or BCMA. When assessing exhaustion, cells were stained with antibodies targeting PD-1, LAG-3, and TIM-3. When assessing phenotype, cells were stained with antibodies targeting CD45RA and CD62L. Accucheck counting beads (Life Technologies) were added to the cells for normalization. Samples were acquired on an LSR II flow cytometer (BD) or an Aurora full-spectrum flow cytometer (Cytek).

Luciferase-based cytotoxicity assay. To determine in vitro CAR T cell cytotoxicity, cell lines (Raji, NALM6, Daudi, Toledo MM. 1S, RPMI8226, U266B1) were transduced with pHIV-Luc-ZsGreen lentivirus and sorted on a FACS Aria flow cytometer (BD) for GFP expression. 3×10⁴ target cells were seeded in wells of a 96-well round bottom plate. CAR T cells from one of three healthy donors were cocultured with target cells at the indicated effector-target ratios and incubated for 16 hours at 37° C., 5% CO₂. Following incubation, 80 μL of supernatant was harvested from each well. Cells were suspended by gentle pipetting, and 100 μL was transferred to a 96-well black flat bottom plate. D-Luciferin (Gold Biotechnology, catalog no. LUCNA-2G) at 150 g/ml was added to the cells and incubated for 5 minutes at 37° C. Luminescence was determined on a Spark multimode plate reader (Tecan).

Serial coculture repeat stimulation assay. To determine the long-term in vitro control and exhaustion of CAR T cells, luciferase-expressing tumor cells were plated at 5×10⁴ cells/well. CAR T cells were cocultured at a defined effector-target ratio and incubated for 24 hours at 37° C., 5% CO₂. Following incubation, cells were transferred to a 96-well black flat bottom plate. D-luciferin was added to cells and luminescence was determined on a Spark multimode plate reader (Tecan). After measuring luminescence, the cells were washed with FACS buffer and stained with Zombie NIR fixable viability dye (Biolegend) and antibodies targeting CD19, HA, PD-1, TIM-3, CD45RA, and CD62L. Accucheck counting beads (Life Technologies) were added to the cells for normalization. Next, remaining CAR cells were pooled together and normalized based on expansion. CAR T cells were redistributed to wells and fresh tumor cells were added to each well. Plates were incubated for 48 hours at 37° C., 5% CO₂. Luminescence measurements and normalization were repeated until a loss of cytotoxicity was observed.

Cystatin A enzyme-linked immunosorbent assay. Cystatin A concentration was assessed using a Human Cystatin A ELISA kit (Invitrogen, catalog no. EH140RB). Total cell lysates were extracted from CAR T cells using radioimmunoprecipitation assay (RIPA) buffer (Thermo) containing protease inhibitor cocktail (Roche). Total protein concentration was determined using Pierce BCA assay (Thermo). Cystatin A levels were determined by enzyme-linked immunosorbent assay (ELISA) according to manufacturer's instructions (Invitrogen) and calculated using standard curve interpolation. Absorbance was measured at the recommended wavelength on a Spark multimode plate reader (Tecan).

Cathepsin B activity assay. Cathepsin B activity was assessed using the InnoZyme Cathepsin B Activity Assay Kit (MilliPore Sigma, catalog no. CBA001). Total cell lysates were extracted from CAR T cells using the provided cell lysis buffer according to manufacturer's instructions. Total protein concentration was determined using Pierce BCA assay (Thermo Fisher Scientific). Cathepsin B activity was determined fluorometrically according to manufacturer's instructions (Calbiochem). Fluorescence was measured on a Spark multimode plate reader (Tecan).

Treatment of CAR T cells with inhibitors. FMC63 CAR T cells were treated with small-molecule inhibitors targeting Actin (inhibitor: Cytochalasin D, Sigma Aldrich catalog no. C8273), Dynamin (inhibitor: DynaSore, Sigma Aldrich catalog no. D7693), Cathepsin B (inhibitor: Ca-074-Me, SelleckChem catalog no. S7420), Clathrin (inhibitor: PitStop, Abcam catalog no. ab120687), or LFA-1 (inhibitor: BI-1950, Boehringer Ingelheim) at the indicated concentrations for one hour. Following treatment, cells were washed with T cell media and centrifuged at 400×g for 5 minutes. To determine the effect of the non-membrane permeable CTSB inhibitor CA-074 (Millipore Sigma, catalog no. 205530), the inhibitor was added directly to the co-culture at the indicated concentrations. As a control, CAR T cells treated with DMSO. CAR T cells were cocultured with CD19-expressing tumor cells for the indicated intervals.

Western blot. Total cell lysates were extracted from CAR T cells using radioimmunoprecipitation assay (RIPA) buffer (Thermo Fisher Scientific) containing protease inhibitor (Roche). Total protein concentration was determined using Pierce BCA assay (Thermo Fisher Scientific). Samples were separated by SDS-polyacrylamide gel electrophoresis, and separated proteins were transferred to nitrocellulose membranes using an iBlot2 transfer system (Thermo Fisher Scientific). Membranes were blocked with 5% nonfat milk-tris-buffered saline and incubated with primary antibodies against CD19, mCSTA, CISH, or β-actin. Membranes were washed and developed using species-specific secondary anti-IgG/horseradish peroxidase antibodies (R&D Systems) and Western Lightning Plus-ECL solution (PerkinElmer). Bands were visualized and quantified on an iBright 1500 imaging system (Thermo Fisher Scientific).

In vivo cystatin A toxicity. Six- to eight-week-old NOD. Cg-Rag1^{tm1Mom}Il2^{tm1Wjl}/SzJ (NRG) mice (Jackson Laboratory) were irradiated with a sublethal dose of 200 cGy (Rad-Source 2000) and injected with the indicated number of CAR T cells into the lateral tail vein on the same day. To assess toxicity, animals were weighed daily and monitored for signs of distress in accordance with institutional regulations. After 14 days, the mice were euthanized, and the lungs, spleens, liver, and gastrointestinal organs were collected, sectioned, and subjected to H&E staining to assess organ structure. Slides were imaged using a BZ-X810 fluorescence microscope (Keyence) and BZ-X800 analyzer.

Secretome analysis. CAR and CAR_{CSTA} secretomes were analyzed by Codeplex assay (IsoPlexis). Following production and cryopreservation, CAR and CAR_{CSTA} T cells produced from three healthy donors were thawed and cultured in T-cell media+40 IU IL-2 for 48 hours. Cells were activated using anti-CD3/CD28 activation beads (Thermo Fisher) and incubated for 24 hours. Supernatants were collected after 24 hours, and cytokine profiling was measured using IsoPlexis Codeplex (Kcas Bio).

Bulk RNA sequencing. Following production and cryopreservation, CAR and CAR_{CSTA} T cells from two independent productions were thawed and cultured in T-cell media+40 IU/ml IL-2 for 48 hours. After 48 hours, CAR T cells were washed with FACS buffer, stained with anti-CD3, anti-HA, and DAPI, and sorted for CAR expression. Sorted CAR T cells were then pelleted and flash frozen. RNA was extracted, polyA-enriched, and sequenced using Illumina NovaSeq S4 PE100 sequencing targeting 10 million read pairs per sample.

In vivo cystatin A model of CAR T-cell tumor control and persistence. Six- to eight-week-old male NOD. Cg-Rag1^{tm1Mom}Il2rg^{tm1Wjl}/SzJ (NRG) mice (Jackson Laboratory) were irradiated with a sublethal dose of 200 cGy (Day 0). The starting group size (n=5) was based on a power calculation assuming a minimum effect size of 17%, a standard deviation/variance of 10%, and an alpha level of 0.05, according to the following formula:

$$n = \frac{( {σ}_{1}^{2} + {σ}_{2}^{2} ) {( z 1 - α / 2 + z 1 - β )}^{2}}{{Δ}^{2}}$$

n: sample size; σ_{1/2}: variance for each mean; z: critical Z value for a given α/β; α: probability of type I error; β: probability of type II error; Δ: difference between means.

After irradiation, mice were injected with 4×10⁵ NALM6 tumor cells via tail vein injection. On Day 4, mice were injected with 1×10⁶ FMC63 CAR or CAR_{CSTA} T cells or CAR T cells lacking a binding domain (ΔscFv) via tail vein injection. Animals were weighed twice weekly and monitored for signs of distress in accordance with institutional regulations. Tumor burden was assessed weekly in the prone and supine positions by an in vivo imaging system (IVIS). For in vivo imaging, mice received an intraperitoneal injection of 3.3 mg D-luciferin (GOLDBIO #LUCK-10G). On day 28, animals were euthanized, and tissues were collected for analysis by flow cytometry. Average radiance values (p/s/cm²/sr) were determined using Living Image 4.8 software (PerkinElmer).

In vivo model of CAR T-cell solid tumor infiltration. Six- to eight-week-old NOD. Cg-Rag1^{tm1Mom}Il2rg^{tm1Wjl}/SzJ (NRG) mice (Jackson Laboratory) were irradiated with a sublethal dose of 200 cGy (Rad-Source 2000) and injected intratibially on the same day with the indicated number of luciferase-expressing A673 cells (A673-Fluc) expressing CD19-GFP. The starting group size (n=3) was based on a power calculation using the formula shown above, assuming a minimum effect size of 25%, a standard deviation of 10%, and an alpha level of 0.05. One animal was not available for analysis due to insufficient tumor growth. On day 14 after tumor cell injection, the indicated number of ΔscFv, FMC63 CISH^{WT} CAR, or CISH^{KO} CAR_{CSTA} T cells was injected into the lateral tail vein. Four days after CAR T-cell injection, mice were euthanized, and tumors were collected and dissociated using the Human Tumor Dissociation Kit (Miltenyi Biotec, cat #130-095-929) and the OctoMACS tissue dissociator (Miltenyi Biotec). CAR T cells were quantified and phenotyped by flow cytometry.

In vivo model of CAR T-cell solid tumor control. Six- to eight-week-old NOD. Cg-Rag1^{tm1Mom}Il2rg^{tm1Wjl}/SzJ (NRG) mice (Jackson Laboratory) were irradiated with a sublethal dose of 200 cGy (Rad-Source 2000) and injected intratibially on the same day with the indicated number of luciferase-expressing A673 cells (A673-Fluc) expressing CD19-GFP. The starting group sizes (n=4-5) were based on a power calculation using the formula shown above, assuming a minimum effect size of₁₇-2₀% between the respective groups, a standard deviation of 10%, and an alpha level of 0.05. Four animals were not available for analysis due to unscheduled deaths. On day 7 after tumor cell injection, the indicated number of ΔscFv, FMC63 CISH^{WT} CAR, or CISH^{KO} CAR_{CSTA} T cells was injected into the lateral tail vein. Tumor burden was assessed weekly by an in vivo imaging system (IVIS). For in vivo imaging, mice received an intraperitoneal injection of 3.3 mg D-luciferin (GOLDBIO #LUCK-10G). Peripheral blood was collected on day 19 after CAR T-cell injection for CAR T-cell quantification and phenotyping by flow cytometry.

Statistical Analysis. The respective statistical tests are stated in the figure legends. Generally, statistical significance between two groups was determined by two-sided Student's t-test or Mann-Whitney U test. Statistical significance between groups of three or more was determined by one- or two-way analysis of variance (ANOVA). Significance of differences between cell numbers, mean fluorescence intensity, and area under curve, was calculated by two-sided Student's t test. Significance of differences in murine tumor control was determined by two-way ANOVA. All statistical tests were performed using Prism 10 (GraphPad). Results were considered significant when p<0.05; * =p<0.05; ** =p<0.01; *** =p<0.001; **** =p<0.0001.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Ferlay, J. et al. Cancer statistics for the year 2020: An overview. International Journal of Cancer 149, 778-789 (2021).
2. Maude, S. L. et al. Tisagenlecleucel in Children and Young Adults with B-Cell Lymphoblastic Leukemia. N. Engl. J. Med. 378, 439-448 (2018).
3. Purdum, A., Tieu, R., Reddy, S. R. & Broder, M. S. Direct Costs Associated with Relapsed Diffuse Large B-Cell Lymphoma Therapies. The Oncologist 24, 1229-1236 (2019).
4. Kanas, G. et al. Epidemiology of diffuse large B-cell lymphoma (DLBCL) and follicular lymphoma (FL) in the United States and Western Europe: population-level projections for 2020-2025. Leukemia & Lymphoma 63, 54-63 (2022).
5. Olson, M. L. et al. Low-affinity CAR T cells exhibit reduced trogocytosis, preventing rapid antigen loss, and increasing CAR T cell expansion. Leukemia 36, 1943-1946 (2022).
6. Hamieh, M. et al. CAR T cell trogocytosis and cooperative killing regulate tumour antigen escape. Nature 568, 112-116 (2019).
7. Li, Y. et al. KIR-based inhibitory CARs overcome CAR-NK cell trogocytosis-mediated fratricide and tumor escape. Nat Med 28, 2133-2144 (2022).
8. Feins, S., Kong, W., Williams, E. F., Milone, M. C. & Fraietta, J. A. An introduction to chimeric antigen receptor (CAR) T-cell immunotherapy for human cancer. American Journal of Hematology 94, S3-S9 (2019).
9. Tyagarajan, S., Spencer, T. & Smith, J. Optimizing CAR-T Cell Manufacturing Processes during Pivotal Clinical Trials. Mol Ther Methods Clin Dev 16, 136-144 (2019).
10. CAR T Cells: Engineering Patients' Immune Cells to Treat Their Cancers. https://www.cancer.gov/about-cancer/treatment/research/car-t-cells (2022).
11. Grover, P. et al. Chimeric antigen receptor T-cell therapy in adults with B-cell acute lymphoblastic leukemia. Blood Adv 6, 1608-1618 (2022).
12. Bethge, W. A. et al. GLA/DRST real-world outcome analysis of CAR T-cell therapies for large B-cell lymphoma in Germany. Blood 140, 349-358 (2022).
13. Shah, N. N. & Fry, T. J. Mechanisms of resistance to CAR T cell therapy. Nat Rev Clin Oncol 16, 372-385 (2019).
14. D'Agostino, M. & Raje, N. Anti-BCMA CAR T-cell therapy in multiple myeloma: can we do better?Leukemia 34, 21-34 (2020).
15. Joly, E. & Hudrisier, D. What is trogocytosis and what is its purpose?Nat Immunol 4, 815-815 (2003).
16. Dixon, A. S. et al. NanoLuc Complementation Reporter Optimized for Accurate Measurement of Protein Interactions in Cells. ACS Chem. Biol. 11, 400-408 (2016).
17. Miyake, K. et al. Trogocytosis of peptide-MHC class II complexes from dendritic cells confers antigen-presenting ability on basophils. Proc Natl Acad Sci USA 114, 1111-1116 (2017).
18. Willinger, T., Staron, M., Ferguson, S. M., Camilli, P. D. & Flavell, R. A. Dynamin 2-dependent endocytosis sustains T-cell receptor signaling and drives metabolic reprogramming in T lymphocytes. Proc. Natl. Acad. Sci. 112, 4423-4428 (2015).
19. Shi, Q. et al. Increased glucose metabolism in TAMs fuels O-GlcNAcylation of lysosomal Cathepsin B to promote cancer metastasis and chemoresistance. Cancer Cell 40, 1207-1222.e10 (2022).
20. Yang, L. et al. Histone deacetylase 3 contributes to the antiviral innate immunity of macrophages by interacting with FOXK1 to regulate STAT1/2 transcription. Cell Rep. 38, 110302 (2022).
21. Hook, G. et al. Cathepsin B Gene Knockout Improves Behavioral Deficits and Reduces Pathology in Models of Neurologic Disorders. Pharmacol. Rev. 74, 600-629 (2022).
22. Cavallo-Medved, D., Moin, K. & Sloane, B. Cathepsin B. AFCS Nat Mol Pages 2011, A000508 (2011).
23. Balaji, K. N., Schaschke, N., Machleidt, W., Catalfamo, M. & Henkart, P. A. Surface Cathepsin B Protects Cytotoxic Lymphocytes from Self-destruction after Degranulation. Journal of Experimental Medicine 196, 493-503 (2002).
24. Turk, V., Turk, B. & Turk, D. Lysosomal cysteine proteases: facts and opportunities. The EMBO Journal 20, 4629-4633 (2001).
25. Olson, O. C. & Joyce, J. A. Cysteine cathepsin proteases: regulators of cancer progression and therapeutic response. Nat Rev Cancer 15, 712-729 (2015).
26. Gabrijelcic, D. et al. Cathepsins B, H and L in human breast carcinoma. Eur J Clin Chem Clin Biochem 30, 69-74 (1992).
27. Zhong, L. et al. Small molecules in targeted cancer therapy: advances, challenges, and future perspectives. Signal Transduct. Target. Ther. 6, 201 (2021).
28. Du, J. et al. Cutaneous toxicity of FDA-approved small-molecule kinase inhibitors. Expert Opin. Drug Metab. Toxicol. 17, 1311-1325 (2021).
29. Pavlova, A. & Björk, I. Grafting of Features of Cystatins C or B into the N-Terminal Region or Second Binding Loop of Cystatin A (Stefin A) Substantially Enhances Inhibition of Cysteine Proteinases. Biochemistry 42, 11326-11333 (2003).
30. Estrada, S. et al. The Role of Gly-4 of Human Cystatin A (Stefin A) in the Binding of Target Proteinases. Characterization by Kinetic and Equilibrium Methods of the Interactions of Cystatin A Gly-4 Mutants with Papain, Cathepsin B, and Cathepsin L. Biochemistry 37, 7551-7560 (1998).
31. Pol, E. & Björk, I. Role of the single cysteine residue, Cys 3, of human and bovine cystatin B (stefin B) in the inhibition of cysteine proteinases. Protein Sci. 10, 1729-1738 (2001).
32. Bervar, A. et al. Invasiveness of Transformed Human Breast Epithelial Cell Lines Is Related to Cathepsin B and Inhibited by Cysteine Proteinase Inhibitors. Biol. Chem. 384, 447-455 (2003).
33. Hentze, H., Lin, X. Y., Choi, M. S. K. & Porter, A. G. Critical role for cathepsin B in mediating caspase-1-dependent interleukin-18 maturation and caspase-1-independent necrosis triggered by the microbial toxin nigericin. Cell Death Differ. 10, 956-968 (2003).
34. Ochieng, J. & Chaudhuri, G. Cystatin Superfamily. J. Heal. Care Poor Underserved 21, 51-70 (2010).
35. López-Cantillo, G., Urueña, C., Camacho, B. A. & Ramirez-Segura, C. CAR-T Cell Performance: How to Improve Their Persistence?Front. Immunol. 13, 878209 (2022).
36. Louis, C. U. et al. Antitumor activity and long-term fate of chimeric antigen receptor-positive T cells in patients with neuroblastoma. Blood 118, 6050-6056 (2011).
37. Jafarzadeh, L., Masoumi, E., Fallah-Mehrjardi, K., Mirzaei, H. R. & Hadjati, J. Prolonged Persistence of Chimeric Antigen Receptor (CAR) T Cell in Adoptive Cancer Immunotherapy: Challenges and Ways Forward. Front Immunol 11, 702 (2020).
38. Sterner, R. C. & Sterner, R. M. CAR-T cell therapy: current limitations and potential strategies. Blood Cancer J. 11, 1-11 (2021).
39. Arcangeli, S. et al. CAR T cell manufacturing from naive/stem memory T lymphocytes enhances antitumor responses while curtailing cytokine release syndrome. J Clin Invest 132, (2022).
40. Busch, D. H., Fräßle, S. P., Sommermeyer, D., Buchholz, V. R. & Riddell, S. R. Role of memory T cell subsets for adoptive immunotherapy. Seminars in Immunology 28, 28-34 (2016).
41. Künkele, A. et al. Functional Tuning of CARs Reveals Signaling Threshold above Which CD8+ CTL Antitumor Potency Is Attenuated due to Cell Fas-FasL-Dependent AICD. Cancer Immunology Research 3, 368-379 (2015).
42. Rolle, C. E., Carrio, R. & Malek, T. R. Modeling the CD8+ T Effector to Memory Transition in Adoptive T-Cell Antitumor Immunotherapy. Cancer Research 68, 2984-2992 (2008).
43. Labanieh, L. et al. Enhanced safety and efficacy of protease-regulated CAR-T cell receptors. Cell 185, 1745-1763.e22 (2022).
44. Vander Mause et al., 2023; DOI: 10.1126/scitranslmed.add7900.
45. Sadelain, M. CD19 CAR T Cells. Cell 171, 1471 (2017).
46. Maude, S. L. et al. Tisagenlecleucel in Children and Young Adults with B-Cell Lymphoblastic Leukemia. N. Engl. J. Med. 378, 439-448 (2018).
47. Sadelain, M., Rivière, I. & Riddell, S. Therapeutic T cell engineering. Nature 545, 423-431 (2017).
48. Raje, N. et al. Anti-BCMA CAR T-Cell Therapy bb2121 in Relapsed or Refractory Multiple Myeloma. N. Engl. J. Med. 380, 1726-1737 (2019).
49. D'Agostino, M. & Raje, N. Anti-BCMA CAR T-cell therapy in multiple myeloma: can we do better?Leukemia 34, 21-34 (2020).
50. Romanski, A. et al. CD19-CAR engineered NK-92 cells are sufficient to overcome NK cell resistance in B-cell malignancies. J. Cell. Mol. Med. 20, 1287-1294 (2016).
51. Boissel, L. et al. Retargeting NK-92 cells by means of CD19- and CD20-specific chimeric antigen receptors compares favorably with antibody-dependent cellular cytotoxicity. OncoImmunology 2, e26527 (2013).
52. Hamieh, M. et al. CAR T cell trogocytosis and cooperative killing regulate tumour antigen escape. Nature 568, 112-116 (2019).
53. Olson, M. L. et al. Low-affinity CAR T cells exhibit reduced trogocytosis, preventing rapid antigen loss, and increasing CAR T cell expansion. Leukemia 36, 1943-1946 (2022).
54. Li, Y. et al. KIR-based inhibitory CARs overcome CAR-NK cell trogocytosis-mediated fratricide and tumor escape. Nat Med 28, 2133-2144 (2022).
55. Camviel, N. et al. Both APRIL and antibody-fragment-based CAR T cells for myeloma induce BCMA downmodulation by trogocytosis and internalization. J. Immunother. Cancer 10, e005091 (2022).
56. Cohen, A. D. et al. B cell maturation antigen-specific CAR T cells are clinically active in multiple myeloma. J. Clin. Investig. 129, 2210-2221 (2019).
57. Radhakrishnan, S. V. et al. CD229 CAR T cells eliminate multiple myeloma and tumor propagating cells without fratricide. Nat. Commun. 11, 798 (2020).
58. Marei, H. et al. Antibody targeting of E3 ubiquitin ligases for receptor degradation. Nature 610, 182-189 (2022).
59. Békés, M., Langley, D. R. & Crews, C. M. PROTAC targeted protein degraders: the past is prologue. Nat. Rev. Drug Discov. 21, 181-200 (2022).
60. Brooks, S. R., Kirkham, P. M., Freeberg, L. & Carter, R. H. Binding of Cytoplasmic Proteins to the CD19 Intracellular Domain Is High Affinity, Competitive, and Multimeric. J. Immunol. 172, 7556-7564 (2004).
61. Ruella, M. et al. Induction of resistance to chimeric antigen receptor T cell therapy by transduction of a single leukemic B cell. Nat. Med. 24, 1499-1503 (2018).
62. Dixon, A. S. et al. NanoLuc Complementation Reporter Optimized for Accurate Measurement of Protein Interactions in Cells. ACS Chem. Biol. 11, 400-408 (2016).
63. Miyake, K. et al. Trogocytosis of peptide-MHC class II complexes from dendritic cells confers antigen-presenting ability on basophils. Proc Natl Acad Sci USA 114, 1111-1116 (2017).
64. Gilmartin, A. A., Ralston, K. S. & Petri, W. A. Inhibition of Amebic Cysteine Proteases Blocks Amebic Trogocytosis but Not Phagocytosis. J. Infect. Dis. 221, 1734-1739 (2020).
65. Reed, J., Reichelt, M. & Wetzel, S. A. Lymphocytes and Trogocytosis-Mediated Signaling. Cells 10, 1478 (2021).
66. Hudrisier, D., Aucher, A., Puaux, A.-L., Bordier, C. & Joly, E. Capture of Target Cell Membrane Components via Trogocytosis Is Triggered by a Selected Set of Surface Molecules on T or B Cells. J. Immunol. 178, 3637-3647 (2007).
67. Cavallo-Medved, D., Moin, K. & Sloane, B. Cathepsin B. AFCS Nat Mol Pages 2011, A000508 (2011).
68. Mort, J. S. & Buttle, D. J. Cathepsin B. Int. J. Biochem. Cell Biol. 29, 715-720 (1997).
69. Vidak, E., Javoršek, U., Vizovišek, M. & Turk, B. Cysteine Cathepsins and Their Extracellular Roles: Shaping the Microenvironment. Cells 8, 264 (2019).
70. Balaji, K. N., Schaschke, N., Machleidt, W., Catalfamo, M. & Henkart, P. A. Surface Cathepsin B Protects Cytotoxic Lymphocytes from Self-destruction after Degranulation. Journal of Experimental Medicine 196, 493-503 (2002).
71. Olson, O. C. & Joyce, J. A. Cysteine cathepsin proteases: regulators of cancer progression and therapeutic response. Nat Rev Cancer 15, 712-729 (2015).
72. Gabrijelcic, D. et al. Cathepsins B, H and L in human breast carcinoma. Eur J Clin Chem Clin Biochem 30, 69-74 (1992).
73. Estrada, S. et al. The Role of Gly-4 of Human Cystatin A (Stefin A) in the Binding of Target Proteinases. Characterization by Kinetic and Equilibrium Methods of the Interactions of Cystatin A Gly-4 Mutants with Papain, Cathepsin B, and Cathepsin L. Biochemistry 37, 7551-7560 (1998).
74. Pavlova, A., Krupa, J. C., Mort, J. S., Abrahamson, M. & Björk, I. Cystatin inhibition of cathepsin B requires dislocation of the proteinase occluding loop. Demonstration by release of loop anchoring through mutation of His110. FEBS Lett. 487, 156-160 (2000).
75. Pavlova, A. & Björk, I. Grafting of Features of Cystatins C or B into the N-Terminal Region or Second Binding Loop of Cystatin A (Stefin A) Substantially Enhances Inhibition of Cysteine Proteinases. Biochemistry 42, 11326-11333 (2003).
76. Jenko, S. et al. Different propensity to form amyloid fibrils by two homologous proteins—Human stefins A and B: Searching for an explanation. Proteins: Struct., Funct., Bioinform. 55, 417-425 (2004).
77. Xu, X. et al. Mechanisms of Relapse After CD19 CAR T-Cell Therapy for Acute Lymphoblastic Leukemia and Its Prevention and Treatment Strategies. Front. Immunol. 10, 2664 (2019).
78. Mause, E. R. V. et al. Systematic single amino acid affinity tuning of CD229 CAR T cells retains efficacy against multiple myeloma and eliminates on-target off-tumor toxicity. Sci. Transl. Med. 15, eadd7900 (2023).
79. Veatch, S. L. et al. Correlation Functions Quantify Super-Resolution Images and Estimate Apparent Clustering Due to Over-Counting. PLoS ONE 7, e31457 (2012).
80. Lee, D. W. et al. T cells expressing CD19 chimeric antigen receptors for acute lymphoblastic leukaemia in children and young adults: a phase 1 dose-escalation trial. Lancet 385, 517-528 (2015).
81. Ghorashian, S. et al. Enhanced CAR T cell expansion and prolonged persistence in pediatric patients with ALL treated with a low-affinity CD19 CAR. Nat Med 25, 1408-1414 (2019).
82. Song, D.-G. et al. A fully human chimeric antigen receptor with potent activity against cancer cells but reduced risk for off-tumor toxicity. Oncotarget 6, 21533-21546 (2015).
83. Organization, W. H. INN Database. https://extranet.who.int/soinn/.
84. Morales, E. A. et al. Restricting CAR T Cell Trafficking Expands Targetable Antigen Space. bioRxiv 2024.02.08.579002 (2024) doi:10.1101/2024.02.08.579002.

What is claimed is:

1. A population of chimeric antigen receptor (CAR) expressing T cells (CAR T cells) that exhibits reduced trogocytosis, wherein the CAR T cells express or overexpress an inhibitor of Cathepsin B.

2. The population of CAR T cells of claim 1, wherein the inhibitor is selected from the group consisting of a protein, a peptide, and a small molecule inhibitor.

3. The population of CAR T cells of claim 2, wherein the CAR T cells overexpress one or more of Cystatin A, a functional fragment of Cystatin A, Cystatin B, a functional fragment of Cystatin B, Ca-074, Ca-074-Me and E-64D.

4. The population of CAR T cells of claim 3, wherein the functional fragment of Cystatin A is Cystatin A₁₋₅₇.

5. A population of CAR T cells that exhibits reduced surface expression of cancer antigens, wherein the CAR T cells express or overexpress an effector of ubiquitylation.

6. The population of CAR T cells of claim 5, wherein the effector of ubiquitylation is a binding protein linking a transferred antigen to an ubiquitin ligase.

7. The population of CAR T cells of claim 6, wherein the binding protein is selected from the group consisting of a monobody, scFv, DARPIN and a natural ligand of the transferred antigen

8. The population of CAR T cells of claim 6, wherein the ubiquitin ligase is selected from the group consisting of Von-Hippel-Lindau factor, cereblon, damage binding protein 1, and NSlmb.

9. A population of CAR T cells that exhibits both reduced trogocytosis and reduced surface expression of cancer antigens, wherein the CAR T cells express or overexpress an inhibitor of Cathepsin B and express or overexpress an effector of ubiquitylation

10. The population of CAR T cells of claim 9, wherein the inhibitor is selected from the group consisting of a protein, a peptide, and a small molecule inhibitor.

11. The population of CAR T cells of claim 10, wherein the Cathepsin B inhibitor is one or more of Cystatin A, a functional fragment of Cystatin A, Cystatin B, a functional fragment of Cystatin B, Ca-074, Ca-074-Me and E-64D.

12. The population of CAR T cells of claim 11, wherein the functional fragment of Cystatin A is Cystatin A₁₋₅₇.

13. The population of CAR T cells of claim 9, wherein the effector of ubiquitylation is a binding protein linking a transferred antigen to an ubiquitin ligase.

14. The population of CAR T cells of claim 13, wherein the binding protein is selected from the group consisting of a monobody, scFv, DARPIN and a natural ligand of the transferred antigen

15. The population of CAR T cells of claim 13, wherein the ubiquitin ligase is selected from the group consisting of Von-Hippel-Lindau factor, cereblon, damage binding protein 1, and NSlmb.

16. A method of treating cancer in a subject comprising (a) contacting a population of CAR T cells with a Cathepsin B inhibitor and (b) administering the cells of (a) to a subject having cancer.

17. A method of treating cancer in a subject comprising administering a Cathepsin B inhibitor and a population of CAR T cells to a subject having cancer.

18. A method of augmenting anti-cancer activity of CAR T cells in a subject comprising administering a Cathepsin B inhibitor to a subject undergoing CAR T cell therapy.

19. A method of inhibiting trogocytosis in CAR T cells in a subject comprising administering a Cathepsin B inhibitor to a subject undergoing CAR T cell therapy.

20. A method of treating cancer in a subject comprising administering a population of CAR T cells genetically modified to express or overexpress a Cathepsin B inhibitor to a subject having cancer.

21. The method of any one of claims 16-20, wherein the Cathepsin B inhibitor is one or more of Cystatin A, a functional fragment of Cystatin A, Cystatin B, a functional fragment of Cystatin B, Ca-074, Ca-074-Me and E-64D.

22. The method of claim 21, wherein the functional fragment of Cystatin A is Cystatin A₁₋₅₇.

23. A method of treating cancer in a subject comprising administering a population of CAR T cells genetically modified to express or overexpress an effector of ubiquitylation to a subject having cancer.

24. The method of claim 23, wherein the effector of ubiquitylation is a binding protein linking a transferred antigen to an ubiquitin ligase.

25. The method of claim 24, wherein the binding protein is selected from the group consisting of a monobody, scFv, DARPIN and a natural ligand of the transferred antigen

26. The method of claim 24, wherein the ubiquitin ligase is selected from the group consisting of Von-Hippel-Lindau factor, cereblon, damage binding protein 1, and NSlmb.

27. A method of treating cancer in a subject comprising administering a population of CAR T cells genetically modified to express or overexpress a Cathepsin B inhibitor and genetically modified to express or overexpress an effector of ubiquitylation to a subject having cancer.

28. The method of claim 27, wherein the Cathepsin B inhibitor is one or more of Cystatin A, a functional fragment of Cystatin A, Cystatin B, a functional fragment of Cystatin B, Ca-074, Ca-074-Me and E-64D.

29. The method of claim 28, wherein the functional fragment of Cystatin A is Cystatin A₁₋₅₇.

30. The method of claim 27, wherein the effector of ubiquitylation is a binding protein linking a transferred antigen to an ubiquitin ligase.

31. The method of claim 30, wherein the binding protein is selected from the group consisting of a monobody, scFv, DARPIN and a natural ligand of the transferred antigen

32. The method of claim 30, wherein the ubiquitin ligase is selected from the group consisting of Von-Hippel-Lindau factor, cereblon, damage binding protein 1, and NSlmb.

33. A method of inhibiting trogocytosis in a population of CAR T cells comprising contacting the CAR T cells with a Cathepsin B inhibitor.

34. A method of decreasing trogocytosis in a population of CAR T cells comprising genetically modifying the population of CAR T cells to express or overexpress a Cathepsin B inhibitor.

35. A method of decreasing trogocytosis in a population of CAR T cells comprising genetically modifying the population of CAR T cells to express or overexpress an effector of ubiquitylation.

36. A method of decreasing trogocytosis in a population of CAR T cells comprising (i) genetically modifying the population of CAR T cells to express or overexpress a Cathepsin B inhibitor and (ii) genetically modifying the population of CAR T cells to express or overexpress an effector of ubiquitylation.

37. A method of generating trogocytosis-resistant CAR T cells comprising genetically modifying the population of CAR T cells to express or overexpress a Cathepsin B inhibitor.

38. A method of generating trogocytosis-resistant CAR T cells comprising genetically modifying the population of CAR T cells to express or overexpress an effector of ubiquitylation.

39. A method of generating trogocytosis-resistant CAR T cells comprising (i) genetically modifying the population of CAR T cells to express or overexpress a Cathepsin B inhibitor and (ii) genetically modifying the population of CAR T cells to express or overexpress an effector of ubiquitylation.

40. A method of decreasing surface expression of cancer antigens resulting from trogocytosis in a population of CAR T cells comprising genetically modifying the population of CAR T cells to express or overexpress an effector of ubiquitylation.

41. A method of targeted degradation of antigen transferred to CAR T cells via trogocytosis comprising genetically modifying the population of CAR T cells to express or overexpress an effector of ubiquitylation.

42. The method of any one of claims 33-41, wherein the Cathepsin B inhibitor is one or more of Cystatin A, a functional fragment of Cystatin A, Cystatin B, a functional fragment of Cystatin B, Ca-074, Ca-074-Me and E-64D.

43. The method of claim 42, wherein the functional fragment of Cystatin A is Cystatin A₁₋₅₇.

44. The method of any one of claims 33-41, wherein the effector of ubiquitylation is a binding protein linking a transferred antigen to an ubiquitin ligase.

45. The method of claim 44, wherein the binding protein is selected from the group consisting of a monobody, scFv, DARPIN and a natural ligand of the transferred antigen

46. The method of claim 44, wherein the ubiquitin ligase is selected from the group consisting of Von-Hippel-Lindau factor, cereblon, damage binding protein 1, and NSlmb.

47. A method of assaying for inhibitors of trogocytosis, said method comprising:
(a) genetically modifying a CAR T cell to express a N-terminal fragment of NanoLuc luciferase (nLuc),
(b) genetically modifying a tumor cell to express a cancer antigen fused to a C-terminal NanoLuc fragment (cLuc),
(c) culturing the cells of (a) and (b) in the presence of a selected inhibitor under conditions promoting trogocytosis, and
(d) assaying the cells of (a) for luminescence.

48. The method of claim 47, wherein when luminescence is detected in the CAR T cells modified to express nLuc, it is determined that the selected inhibitor does not inhibit trogocytosis.

49. The method of claim 47, wherein when luminescence is not detected in the CAR T cells modified to express nLuc, it is determined that the selected inhibitor inhibits trogocytosis.


SEQUENCE LISTING (23 sequences)

SEQ ID NO: 1 - DNA, 2046 bp, synthetic construct
  source 1..2046: mol_type = other DNA
atgtcctggagctgtattatcctgttcctggtggccaccgctacgggtgccatggcagacattcagatgactcagacgaccagctctctgagcgcaagcctgggcgaccgtgtgactatctcctgccgtgcaagccaggacatctctaagtatctcaattggtaccaacagaaacctgatggcaccgtaaaactcctgatctaccacacctctcgtctgcactctggcgtgccgtctcgtttctccggtagcggctccggcaccgactactctctgaccatttccaacctggaacaagaagacatcgcaacctatttctgccaacagggcaataccctgccgtacacctttggcggtggcaccaagctcgaaatcaccggtggcggtggctccggcggtggcggtagcggcggtggcggttctgaagttaaactgcaagaatccggtccgggcctcgttgccccttcccagtctctgagcgttacctgtacggtttctggtgtctctctgccggattatggcgtgagctggattcgtcaaccgcctcgtaaaggtctggaatggctgggtgttatctggggctctgaaacgacctattacaactctgcactgaaatctcgtctgaccatcattaaagacaactccaaatcccaggtgtttctgaaaatgaatagcctgcagaccgatgacaccgctatttactattgcgcgaaacattattactatggcggttcctatgctatggactactggggtcagggtaccagcgttactgtttcctctgcagctgcgtacccgtacgacgttccggattacgccggcgcggctactaccactccggccccgcgtccgccaaccccagctccgactattgccagccagccactgagcctgcgtccagaggcatgccgcccggccgctggtggcgctgtccacacccgtggtctggatttcgcatgcgatatctacatttgggcaccgctcgctggtacgtgtggtgttctgctcctgtctctggtgatcactctgtactgtaaacgtggtcgtaaaaagctcctgtatatcttcaagcagccatttatgcgtccggttcagaccactcaggaagaggacggttgctcctgccgtttcccggaggaagaggaaggcggttgcgaactgcgcgtcaagtttagccgttctgctgacgcaccggcgtacaagcagggtcagaaccagctgtacaatgagctgaatctgggccgccgtgaagagtacgatgtgctcgataaacgccgtggccgtgacccggaaatgggcggtaaaccgcgccgtaagaacccgcaagaaggtctgtacaacgaactgcagaaggacaaaatggctgaagcatatagcgaaatcggtatgaagggcgagcgccgtcgcggcaaaggtcacgacggtctctaccagggtctgtctaccgcaaccaaagatacttacgacgcgctgcatatgcaggctctgccgcctcgtggctctggtgcaaccaacttcagcctgctcaaacaggcaggcgatgtggaagagaaccctggccctgtcatggttttcaccctcgaagatttcgtcggtgactgggaacagaccgctgcatacaacctggatcaggtactggagcaaggtggcgtgagctccctcctgcagaacctggcggtaagcgttaccccgatccagcgtatcgttcgtagcggcgaaaacgcactgaaaattgacatccacgttattatcccgtacgaaggcctgtctgcggaccaaatggcccagattgaggaagtatttaaagtagtgtacccggttgatgaccatcactttaaggtgatcctgccgtacggcacgctggttatcgatggcgtcactccgaacatgctgaactacttcggtcgtccgtatgaaggtattgctgtatttgatggcaagaaaatcactgtgacgggcaccctgtggaacggcaacaaaattatcgatgagcgtctgattacgccggacggttccatgctgtttcgcgttactatcaattcttga

SEQ ID NO: 2 - protein, 681 aa, synthetic construct
MSWSCIILFLVATATGAMADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAYPYDVPDYAGAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPVMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 3 - DNA, 2490 bp, synthetic construct
  source 1..2490: mol_type = other DNA
atggtaagcaaaggtgaggaagacaacatggcgatcattaaggaatttatgcgtttcaaagtgcacatggagggtagcgtaaacggccatgaattcgaaattgaaggtgaaggcgagggtcgtccgtacgagggcacccaaaccgctaagctgaaagtcaccaaaggtggcccactcccgtttgcttgggatatcctgtctccgcagtttatgtatggttccaaggcgtacgtaaaacacccggccgacatccctgactacctgaagctgtccttcccggaaggcttcaaatgggagcgtgtgatgaacttcgaggacggcggtgttgtaaccgtgactcaagattctagcctgcaggatggtgagttcatttacaaagtcaaactgcgtggtaccaacttcccgtctgatggcccggttatgcagaagaaaaccatgggctgggaggcgtcctctgaacgtatgtacccggaagacggtgcgctgaaaggcgaaatcaaacaacgtctgaaactgaaagacggcggtcactatgacgctgaagtgaaaactacctacaaagcgaagaaaccggtgcaactgccgggtgcatataacgtaaatatcaagctggacatcacctctcacaacgaagactatactatcgtcgaacagtacgaacgcgcggaaggccgtcatagcaccggtggcatggacgagctgtataaaggttctggcgcgacgaacttctccctcctgaagcaggctggtgatgtggaggaaaacccgggtcctgtaatgccgcctccgcgcctcctgtttttcctcctgttcctgaccccgatggaagtacgcccagaggaacctctggtggttaaggttgaggaaggtgacaacgcagttctccaatgcctgaagggcaccagcgatggccctactcagcaactgacttggagccgtgaatctccgctgaaaccattcctcaaactgtccctgggtctgccgggtctgggtatccacatgcgcccgctggccatctggctgttcatcttcaatgtatctcaacagatgggcggtttttatctgtgccaaccgggtccgcctagcgaaaaagcgtggcagccgggctggaccgttaacgttgaaggtagcggtgaactgtttcgttggaacgtttctgatctgggcggtctgggctgtggtctgaaaaaccgctcttccgaaggtccgtcttccccgtctggcaaactgatgagcccgaaactgtatgtgtgggcaaaagaccgtcctgaaatctgggagggtgaaccaccgtgtctgcctccgcgtgattctctgaatcagtccctgtcccaggacctgactatggcgccgggtagcactctgtggctgtcttgcggcgtaccaccggattccgtttcccgtggcccgctgtcctggacccacgttcatccaaaaggtccgaaaagcctcctgtccctggaactgaaggatgaccgcccggcgcgcgacatgtgggtgatggaaactggcctgctcctgccgcgtgcgactgctcaggacgccggtaaatattactgccaccgtggcaacctcactatgtctttccatctggaaatcactgcgcgtccggttctgtggcactggctcctgcgtaccggtggctggaaagtgagcgccgtgactctggcttacctgatcttctgcctgtgttccctggttggtatcctgcacctgcagcgcgcactggtcctgcgccgtaagcgcaaacgtatgaccgatccaacccgccgttttttcaaagtaactccgccaccgggctctggtccgcagaaccagtacggcaacgtactgtctctgccgacgccgacctccggcctgggtcgtgcacagcgttgggcagctggtctgggtggcactgcaccgagctacggcaatccgtctagcgacgtgcaagctgacggtgcactgggctctcgttctccgcctggtgtgggtccggaggaagaggaaggtgagggctatgaggaaccggactctgaagaggatagcgaattttatgagaacgacagcaatctgggtcaggaccaactgtcccaagacggtagcggctatgaaaaccctgaagatgaaccgctgggcccagaagacgaggattctttttccaacgctgaatcctacgaaaacgaagacgaggaactgacgcagccggtggcacgtactatggattttctgtctccgcacggttccgcttgggacccgtcccgtgaagctactagcctgggctcccaatcctacgaagacatgcgcggcatcctgtacgcagctcctcaactgcgttctattcgtggtcaaccgggtccaaaccacgaagaggacgcggattcctacgaaaacatggataatccagacggcccggatcctgcatggggtggcggtggccgtatgggtacctggtctacccgtggcggttctgtatccggttggcgtctgttcaagaaaatttcttaa

SEQ ID NO: 4 - protein, 829 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGATNFSLLKQAGDVEENPGPVMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTRGGSVSGWRLFKKIS

SEQ ID NO: 5 - DNA, 1722 bp, synthetic construct
  source 1..1722: mol_type = other DNA
atggtaagcaaaggtgaggaagacaacatggcgatcattaaggaatttatgcgtttcaaagtgcacatggagggtagcgtaaacggccatgaattcgaaattgaaggtgaaggcgagggtcgtccgtacgagggcacccaaaccgctaagctgaaagtcaccaaaggtggcccactcccgtttgcttgggatatcctgtctccgcagtttatgtatggttccaaggcgtacgtaaaacacccggccgacatccctgactacctgaagctgtccttcccggaaggcttcaaatgggagcgtgtgatgaacttcgaggacggcggtgttgtaaccgtgactcaagattctagcctgcaggatggtgagttcatttacaaagtcaaactgcgtggtaccaacttcccgtctgatggcccggttatgcagaagaaaaccatgggctgggaggcgtcctctgaacgtatgtacccggaagacggtgcgctgaaaggcgaaatcaaacaacgtctgaaactgaaagacggcggtcactatgacgctgaagtgaaaactacctacaaagcgaagaaaccggtgcaactgccgggtgcatataacgtaaatatcaagctggacatcacctctcacaacgaagactatactatcgtcgaacagtacgaacgcgcggaaggccgtcatagcaccggtggcatggacgagctgtataaaggttctggcgcgacgaacttctccctcctgaagcaggctggtgatgtggaggaaaacccgggtcctgtaatgccgcctccgcgcctcctgtttttcctcctgttcctgaccccgatggaagtacgcgctcgtccggttctgtggcactggctcctgcgtactggtggctggaaagtttctgcggtgacgctcgcatacctgatcttctgcctgtgttctctggtgggcatcctgcacctgcaacgtgctctggttctgcgtcgcaaacgcaagcgtatgaccgatccgacccgtcgctttttcaaagtaactccgccaccgggtagcggtccacagaaccagtatggcaacgtgctctctctgccgaccccaaccagcggtctgggccgtgctcagcgttgggctgcaggtctgggtggcaccgctccgtcctacggtaacccttcttccgacgttcaagcagacggtgctctgggctctcgctctccgccaggcgtgggcccggaggaagaggaaggtgaaggctacgaggaacctgactccgaggaagactccgaattctacgaaaatgatagcaacctgggtcaggaccagctgtcccaggacggttccggctacgaaaacccggaagacgaacctctgggcccggaggacgaagactctttcagcaacgcggaatcctacgaaaatgaagacgaggaactgacgcagccggtagcgcgtaccatggacttcctgtctccgcacggctctgcctgggaccctagccgtgaagcgacctctctgggcagccagtcttatgaagacatgcgcggtattctctacgctgcaccgcagctgcgtagcattcgcggccagccaggtcctaatcacgaggaagacgctgactcctacgaaaacatggataacccggatggcccagacccggcttggggtggcggtggccgtatgggtacgtggagcacccgtggcggttccgtatccggttggcgtctgttcaaaaagatcagctaa

SEQ ID NO: 6 - protein, 573 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGATNFSLLKQAGDVEENPGPVMPPPRLLFFLLFLTPMEVRARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTRGGSVSGWRLFKKIS

SEQ ID NO: 7 - DNA, 2298 bp, synthetic construct
  source 1..2298: mol_type = other DNA
atgagctggtcatgtattatactgttcctggttgccaccgctaccggcgctatggcggacatacaaatgacccaaactacctcaagcttgagcgcgtctttaggagatcgcgtcactatctcctgtcgcgcatcccaggatatcagtaaatatctgaattggtaccagcagaaacctgatggaacagtcaagctgctgatataccatactagtagacttcactccggagtcccatctaggttctcaggctccgggtccggaaccgactacagcttgaccattagtaatttagagcaggaagacattgcgacttacttttgccaacaagggaacactcttccctatactttcgggggaggcacaaagctcgaaatcactggcggtggcggaagtggtggggggggatctggtggtggtggatccgaggtcaaactgcaggagtcaggacctgggctcgtggccccatcacagtcacttagcgtgacctgtacggtatcaggcgtgtcactgcctgactatggagtgtcttggattagacagccaccccgaaaagggttagagtggctcggcgtgatttggggttctgaaacaacctactataattctgccctcaagtcacggctgacaattataaaagacaatagcaagtctcaggtgttcctgaagatgaacagtttacagaccgacgacaccgcaatctattattgcgccaagcactattactatgggggatcatacgctatggattactggggccagggcacaagcgtcaccgtcagctccgcagcagcttacccgtacgacgtgcctgactacgccggagctgctactaccacacccgctcctagaccccccacccccgcacctacaatcgcctcacagcccctctccttacgtcctgaagcgtgcagacccgcggcagggggcgccgtccacacccgcgggttagactttgcatgcgatatctatatctgggcccctcttgccggtacctgcggtgtgcttctgctgtctctcgtcatcacactgtattgcaagcgtggaaggaagaagctgctctacatctttaaacagcccttcatgaggcccgtgcagacaactcaggaggaggatggctgcagttgtcggtttcccgaagaagaagaggggggatgtgaactgcgagtgaagttttcccgatcagctgatgctcctgcgtataagcagggccagaaccagctgtacaacgagttaaatctcggtaggcgggaggagtacgatgtcctggacaagcgaagaggacgggacccagagatgggagggaaacctcggaggaaaaatccacaggagggtctttacaacgagcttcagaaagacaaaatggctgaggcctacagcgagatcggaatgaaaggcgaacgaaggcgaggaaaaggtcacgacggcttgtatcaggggctgtccacggctaccaaggatacttatgacgctctgcatatgcaggccctgcccccaagaggatccggggagggtaggggttccttgttaacttgcggcgatgtggaggagaatcctggtcctgttatggtattcacactggaggatttcgtcggagattgggagcaaaccgccgcctacaacctcgatcaagtgcttgaacagggcggtgtctcatctttgttacagaacttagccgtgagcgtgaccccaattcagaggatcgtgcgaagcggcgagaatgccctcaagatcgacatacacgttatcatcccttatgagggtctctctgcagatcagatggcgcaaattgaggaggtgtttaaggtcgtttacccagtagatgatcatcatttcaaggtgatcctgccttatggaaccctcgtaatcgatggagtgaccccgaacatgctgaactactttgggagaccatacgaaggtatcgccgtttttgatgggaagaaaattacagtgacaggtaccctttggaatgggaacaagattatcgacgagaggctgatcacacctgacgggagcatgctgtttcgggtgacgatcaattcaggcagcggagcaactaacttttctctcctgaaacaagccggtgacgtcgaggagaatccaggccctatgatccccggcggccttagcgaagcaaaacccgccactcccgaaattcaggagattgtggacaaggtgaagccacagttggaagagaagaccaacgaaacatacggcaagctggaggcagtccaatataaaacacaggtggtcgcaggaactaactattatattaaggtgcgagttcagcacctcctgtag

SEQ ID NO: 8 - protein, 801 aa, synthetic construct
MSWSCIILFLVATATGAMADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAYPYDVPDYAGAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPVMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSGATNFSLLKQAGDVEENPGPVMIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 9 - DNA, 2481 bp, synthetic construct
  source 1..2481: mol_type = other DNA
atgtcttggtcttgtattatcctgtttctggtagcaaccgcaaccggtgcaatggcagatattcaaatgactcagactaccagctctctgtctgcatccctgggtgaccgtgtgactattagctgccgcgcttctcaggacatctccaaatacctgaactggtaccagcaaaaacctgacggcaccgtaaagctcctgatttatcacacctctcgcctgcactccggcgttccgtctcgtttcagcggctccggctctggtaccgattacagcctgaccatcagcaacctggaacaggaagacatcgcgacgtatttctgtcagcaaggtaataccctgccgtatacctttggtggcggtaccaaactggaaatcaccggtggcggtggctccggcggtggcggttccggcggtggcggtagcgaagttaagctccaggaatccggcccgggtctggttgcgccgtcccagtccctgtccgtaacgtgcactgtttccggcgtgtctctgcctgattacggtgtctcttggatccgccagccaccgcgtaaaggcctggaatggctgggcgtcatctggggttctgaaacgacctactataactccgccctgaaaagccgcctgaccattatcaaagacaactccaaatctcaggttttcctgaagatgaactctctgcagactgatgacaccgctatctattactgcgcgaaacactactattacggcggtagctacgccatggattattggggtcagggcacttctgttaccgttagctccgctgcagcgtacccgtatgatgttccagattacgccggcgcggcaaccactaccccggcgccgcgtccaccgactcctgctccgactattgcttcccagccgctgagcctgcgtccggaagcctgccgtccggctgcaggtggcgctgttcacactcgcggtctggatttcgcgtgcgacatctacatctgggctccgctggctggtacgtgcggcgtactcctgctctctctggtgattacgctgtattgcaaacgtggtcgcaagaaactgctctacatcttcaagcagccgtttatgcgtcctgtccagactacccaggaagaggatggttgtagctgtcgttttccggaggaagaggaaggcggttgcgaactgcgcgtgaaattcagccgttctgccgacgcgccggcttacaaacaaggtcagaaccagctgtacaacgagctgaacctgggccgtcgcgaggaatacgatgttctggacaaacgtcgcggccgtgacccagagatgggtggcaaaccgcgccgtaaaaacccgcaggaaggtctctacaacgagctgcagaaggacaagatggccgaagcctactccgagatcggcatgaaaggtgagcgccgtcgcggtaaaggccatgacggtctgtaccagggcctgagcaccgctactaaagatacctatgatgctctgcatatgcaggcactgccaccgcgcggctccggtgagggccgcggttctctgctcacctgtggtgatgttgaagagaacccgggcccagttatggttttcaccctggaagatttcgttggcgactgggaacagactgccgcgtacaacctggatcaggttctggagcagggtggcgtttcctctctgctccagaacctggcggttagcgttaccccgattcaacgtatcgtccgtagcggtgaaaacgctctgaagatcgatattcatgtaattatcccgtacgaaggtctgtctgcagaccagatggcccagattgaggaagtgtttaaggtggtttacccggtggacgatcatcacttcaaagttattctgccgtacggtaccctggttatcgacggtgttactccgaatatgctgaactacttcggtcgtccgtatgaaggtatcgcggtgttcgacggcaagaaaatcaccgtgaccggcaccctgtggaacggtaacaaaattatcgacgagcgtctgatcactccagatggcagcatgctcttccgtgtcaccatcaactctggcagcggtgcaaccaatttttccctcctgaagcaggcaggtgacgtggaggaaaacccgggcccgatgggctccggtgtgaaacagaccctgaacttcgacctgctcaaactggcgggcgatgtagaatccaacccgggccctatgatgtgcggtgcgccgtctgctactcagccggcaactgctgagacccagcacattgcggaccaggttcgttcccagctggaggaaaaagaaaacaagaaattcccggtgttcaaagcagtaagctttaagagccaggtcgtagcaggcaccaactacttcattaaagtgcatgtaggcgacgaagattttgttcacctgcgtgttttccagagcctgccgcacgaaaacaaacctctgaccctgagcaactaccagaccaacaaagcgaaacacgatgagctgacctatttctaa

SEQ ID NO: 10 - protein, 826 aa, synthetic construct
MSWSCIILFLVATATGAMADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAYPYDVPDYAGAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPVMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSGATNFSLLKQAGDVEENPGPVGSGVKQTLNFDLLKLAGDVESNPGPMMCGAPSATQPATAETQHIADQVRSQLEEKENKKFPVFKAVSFKSQVVAGTNYFIKVHVGDEDFVHLRVFQSLPHENKPLTLSNYQTNKAKHDELTYF

SEQ ID NO: 11 - DNA, 2298 bp, synthetic construct; identical to SEQ ID NO: 7
  source 1..2298: mol_type = other DNA

SEQ ID NO: 12 - protein, 765 aa, synthetic construct
MSWSCIILFLVATATGAMADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAYPYDVPDYAGAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPVMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSGATNFSLLKQAGDVEENPGPMIPGGLSEAKPATPEIQEIVDKVKPQLEEKTNETYGKLEAVQYKTQVVAGTNYYIKVRVQHLL

SEQ ID NO: 13 - DNA, 1422 bp, synthetic construct
  source 1..1422: mol_type = other DNA
atgttgcttcttgtaactagcctcctgctgtgcgagctgccgcatcccgccttcctcctgattccctgtcatcccgagtgtcagccgcagaatggcagcgtgacatgtttcggaccagaggccgaccaatgtgtggcctgcgctcattataaagatccacccttttgtgttgcaaggtgtccatccggagtgaaacccgatctgtcttacatgcccatctggaagtttccagacgaggaaggcgcatgtcagccttgccctattaattgtacccattcatgcgtggacttggacgacaaagggtgcccagcagaacagcgtgctagtcccttgacatccatcatctccgcggttgtgggcattctgctcgtggtggtcctcggggtcgtgtttggcatcctgataggttcaggggccactaacttctctctgctgaagcaggcaggggacgttgaggagaacccaggcccaatgaaaatggaaaccgataagattatggatgagactaattctaacgctcaagccttcacaacgacaatgctgtacgaccccgtacggaaaaaagactcatccccaacgtaccagaccgagagggaattatgttttcagtattttacccaatggtccgaaagcggtcaggtagacttcgtggagcacctcttgtctcgcatgtgccactatcagcacggccagataaacgcctatttaaagcccatgttgcagcgcgatttcatcacgctgctcccaatcaaaggactggatcacatcgctgaaaatatactgtcttaccttgacgctgaatcactgaagagttctgagctggtgtgtaaggagtggctgcgggtgatttccgaaggtatgctttggaagaaactgatagaacgcaaggtccggacggactccctgtggaggggactggccgagcgccgcaattggatgcagtatcttttcaaaccaaggcctgggcagacgcagaggccccattcctttcatagagaattattcccaaagataatgaacgatatcgactccattgagaacaactggcgcacaggtcgccatttagaaatggatcaggtgcagctcgttgagtccggaggagctctggtgcagccaggcggaagcctgcgactgtcctgtgcagcgtcagggtttcccgttaataggtatagcatgagatggtatcgccaggcgcctggcaaggaacgcgaatgggttgcaggtatgtcctcagccggcgaccggtcatcctacgaggatagcgtgaagggaaggtttacaatctctcgcgatgacgcccgcaatactgtctatcttcagatgaactccctcaagccagaagataccgcggtctattactgtaacgtgaatgtaggctttgagtactgggggcagggaactcaggtgaccgtgtcttcctaa

SEQ ID NO: 14 - protein, 473 aa, synthetic construct
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGSGATNFSLLKQAGDVEENPGPMKMETDKIMDETNSNAQAFTTTMLYDPVRKKDSSPTYQTERELCFQYFTQWSESGQVDFVEHLLSRMCHYQHGQINAYLKPMLQRDFITLLPIKGLDHIAENILSYLDAESLKSSELVCKEWLRVISEGMLWKKLIERKVRTDSLWRGLAERRNWMQYLFKPRPGQTQRPHSFHRELFPKIMNDIDSIENNWRTGRHLEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 15 - DNA, 2409 bp, synthetic construct
  source 1..2409: mol_type = other DNA
atgccacccccacgacttctgtttttcctgctgtttttgacaccaatggaagttagaccagaagagccattggtggttaaggtcgaagagggcgacaatgcagtgctgcaatgcttaaaaggcacttccgatggacctacgcagcaactcacatggtcccgggagagccccttgaaaccattcctgaagctcagtttgggtctgccaggcctggggattcatatgcgtcctttggcgatttggctgtttatatttaacgtgagccagcagatggggggcttttatctgtgtcagccagggccacccagcgagaaggcttggcagcctgggtggacagtaaatgtggagggtagcggcgaattgttcaggtggaacgtctctgatctggggggtcttggctgcggcctgaagaatagatctagcgagggtcccagtagccctagcggaaagctgatgtccccgaaactttatgtctgggcgaaagaccgccccgagatctgggagggagagcccccctgtctgcctcctcgggatagcttgaaccagagcctgagccaggatttgactatggctcctggctccaccctgtggctgtcttgtggagtgccgccagatagcgtgagccgcgggccactgagttggacacacgtgcacccaaagggaccaaagagcttgctttcactggaactgaaagacgatcgccccgcacgtgatatgtgggttatggagacaggtctgctcctgccacgagccaccgctcaggatgctggaaagtattactgccatcgcgggaatctcaccatgagctttcacctggagatcacagcacgccctgtactgtggcactggctgttgcgaacggggggttggaaagtgtccgccgtcacattagcttatctgattttctgcctgtgttcactcgtggggatactccatctgcagcgtgcgctggtgctgaggcgtaaacggaaacggatgactgacccgacccggcgtttttttaaggtaacacctcctcccgggtcagggccacaaaaccagtacgggaatgtgctgtcacttcctacgcctacttcaggcctcgggcgcgcccaacgttgggcagctggccttggcggaactgcgccaagctacggaaacccgtcatccgatgtgcaggccgatggagcgttgggtagccggagtcctcccggagtgggaccagaagaggaggaaggagaaggttatgaggagcctgattctgaggaagacagcgaattttacgaaaatgacagtaacctgggtcaggaccagcttagccaggacggctccgggtatgagaacccggaggatgagcctttgggccctgaagacgaagacagtttttctaatgctgaaagctacgaaaatgaggacgaggaactgacacagcctgtggctaggacaatggatttcctctcccctcatggaagtgcatgggaccctagtcgcgaggccacttctctcggaagccagtcatacgaggacatgcgtggaatcttgtacgccgcacctcagctgagatccatccgaggtcagccgggccccaaccatgaggaagatgccgactcatacgaaaatatggataacccggacggacccgatccagcctggggcggaggcggaagaatgggaacgtggtccacccgcggcggctcaggggggtcaatggtcagtaagggtgaggaactgtttactggagtagttcctattctcgtggagcttgacggggacgtgaatgggcataagtttagcgtcagcggtgagggagagggagatgccacctatggaaaactgacattgaagtttatatgcaccaccggaaaattgccagtcccttggccaactctcgtgactactttgacatacggcgtccagtgcttttcacgatacccagatcatatgaagcagcacgatttctttaagagcgccatgccagagggatatgtgcaggaacggacaatcttctttaaagacgacggaaactataagaccagggccgaagtgaagttcgaaggagacaccctggtaaatcggatcgagctcaaaggcattgacttcaaagaggacgggaacattcttggtcacaaactcgagtataattacaatagccacaacgtgtatatcatggcagataagcagaagaacggaatcaaagtgaattttaaaatccggcataatatcgaggatgggagcgtgcagctggccgaccactaccaacagaataccccaattggtgacgggcccgtgctcctgcctgataatcactacctctcaactcagtccgctctgtccaaggatccaaatgagaagcgagatcacatggtgctcctggagtttgtgacggcagcaggcattaccctgggaatggatgagctttataaatgataa

SEQ ID NO: 16 - protein, 801 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTRGGSGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - DNA, 3147 bp, synthetic construct
  source 1..3147: mol_type = other DNA
atgagctggtcttgcatcattctgttcctcgtggcgaccgcaactggtgcgatggcagacattcagatgactcagacgaccagctctctgtccgcttctctgggtgatcgtgttactatcagctgccgtgccagccaggacatctccaaatatctgaactggtatcaacagaagccggacggtactgttaaactcctgatttaccacacctcccgcctgcactccggtgtcccgtcccgtttcagcggctctggctctggtactgactactctctcaccatcagcaacctggagcaagaggatatcgcaacgtacttctgccaacagggtaacacgctgccgtacacctttggtggcggtaccaaactggaaattactggcggtggcggttctggtggcggtggcagcggtggcggtggctctgaagttaaactgcaggaatctggcccgggcctggttgctccttcccagtccctgagcgttacctgcactgtttccggcgtttctctgcctgattatggtgtgagctggatccgtcagccgcctcgtaaaggtctggaatggctcggtgttatctggggctctgaaacgacctactataactctgctctgaagagccgtctgaccattatcaaagacaacagcaaatctcaggtattcctcaaaatgaattctctgcaaaccgatgacactgcaatttattactgtgcaaagcactactattacggcggtagctacgcgatggattactggggccagggtacttccgttaccgtgtctagcgcggcagcctatccgtatgatgttccggactacgctggtgcggctactaccactccagcgcctcgcccaccgacccctgcaccgaccatcgcgtctcagccgctgtccctgcgtccggaagcgtgccgtccggccgctggcggtgcggttcacacgcgtggcctcgacttcgcctgcgatatctacatctgggcgcctctggcaggcacctgcggcgtcctgctcctgagcctggttattaccctgtactgcaaacgcggtcgtaagaaactgctctacattttcaaacagccgttcatgcgcccggtgcagaccactcaggaagaggacggctgctcttgtcgtttcccagaggaagaggaaggcggttgcgaactgcgtgtaaaattttcccgcagcgctgatgcgccagcatacaaacagggtcagaatcaactgtataacgagctgaacctgggccgccgtgaagagtacgacgtgctggacaaacgccgtggtcgcgatccagaaatgggtggcaaaccgcgtcgcaaaaatcctcaggaaggcctgtataacgagctgcagaaagacaagatggcggaagcatacagcgaaatcggtatgaaaggtgaacgccgtcgcggtaaaggccatgacggtctgtaccagggcctctctaccgctacgaaagatacctacgatgccctgcatatgcaggcactgcctccgcgtggcagcggcgaaggccgtggctccctcctgacctgtggtgatgtcgaagagaacccaggtccggtaatggtattcaccctggaggattttgttggtgactgggagcagaccgcagcgtacaacctggatcaggttctggaacagggcggtgttagctctctcctgcaaaacctggccgtctctgtcaccccgattcagcgtatcgtacgcagcggtgagaacgctctcaaaattgacatccatgtaattatcccttacgaaggtctgtctgccgaccagatggctcaaatcgaggaagtttttaaagttgtgtacccggttgacgatcaccatttcaaagtcattctgccgtatggcaccctggtcatcgatggtgttaccccgaatatgctgaactattttggtcgtccgtacgaaggtatcgcggttttcgacggcaagaaaatcaccgttacgggtactctgtggaacggtaacaaaatcattgacgaacgcctgatcactccggacggttccatgctgttccgcgtaaccatcaacagcggctctggcgccaccaacttcagcctcctgaaacaagcgggcgatgtggaggaaaacccgggcccggttatggcaaaactgaacaccctggaaaccgaagagtggtttttcaaggatatcacgcgcaaagatgcggaacgtcagctcctggccccaggtaacagcgcgggcgctttcctgatccgtgaatccgagacgctgaaaggtagcttctctctgtctgttcgtgatttcgatccggtacacggcgacgttatcaaacactataaaatccgttccctggacaatggcggttattacatttccccgcgtattaccttcccgtgcatctctgacatgatcaagcactaccagaaacaggcggatggtctgtgtcgtcgcctggaaaaagcgtgcatctctcctaaaagcggtgaacctacgaccgaagacctgtatttccagtccgataacgcgatcgctatgccgcgccgtgccgagaactgggatgaagcagaagttggcgctgaggaagcgggtgttgaggaatacggtccagaggaagatggtggcgaagagagcggtgcagaggaatccggtccagaggaaagcggcccagaggaactgggtgcggaagaggaaatggaagctggccgcccgcgtccggtactgcgctctgtaaacagccgcgaaccgagccaggtcatcttctgcaaccgttccccacgtgtggttctgccggtttggctgaacttcgatggtgaaccgcaaccatacccgaccctgccgccaggcaccggtcgtcgcattcactcttaccgtggccatctgtggctgttccgcgatgcgggcacccatgacggtctcctggttaaccagaccgaactgttcgttccgtctctgaacgttgatggtcaaccgatcttcgcgaacattaccctgccggtttacaccctgaaagaacgttgcctgcaggtggttcgttccctggtgaaaccggaaaattaccgtcgcctcgacatcgtacgtagcctgtatgaggatctggaagatcacccgaatgtccagaaagatctcgaacgcctgacccaggaacgtatcgcccaccagcgtatgggtgatgtgtaa

SEQ ID NO: 18 - protein, 1048 aa, synthetic construct
MSWSCIILFLVATATGAMADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAYPYDVPDYAGAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPVMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSGATNFSLLKQAGDVEENPGPVMAKLNTLETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKSGEPTTEDLYFQSDNAIAMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDV

SEQ ID NO: 19 - DNA, 2235 bp, synthetic construct
  source 1..2235: mol_type = other DNA
atgccccaaaagccgtgggataaggacgcatgggaaatcccaagagaatctattaagctggttaaacgacttggcgctgggcagtttggagaggtgtggatgggctactacaataacagcactaaggtggccgtcaagacactcaagcccggcacaatgagtgttcaggcgttcttagaagaggccaatctcatgaaaaccttacagcatgacaagttagtgcgcctttatgctgtggtgacccgcgaggagccaatctatatcatcaccgaatatatggctaagggcagtcttcttgatttcttaaagagtgacgaaggtggtaaagtgttactgcctaagcttatagacttcagtgcccaaatcgccgaagggatggcctacatcgagaggaaaaactacattcacagggatctgcgcgcggcaaacgtgctcgtcagcgaaagcctcatgtgtaaaattgccgattttgggctggctagagtgatcgaggataacgaatatacagctcgggagggggccaagtttcccatcaagtggacggcgccagaagcgattaattttggttgtttcacaatcaaatccgacgtgtggtccttcggaatccttctgtatgagattgtgacctacggtaaaatcccatatcccggaagaacgaacgcagacgtaatgacagcactcagccagggataccgcatgccaagggtagagaactgtcctgacgaactctatgacataatgaagatgtgctggaaagaaaaggccgaagagcgtccaactttcgattatttgcaatctgtgctggatgacttttacacagccaccgagggccaatatcagcagcagcctagcggcgagccgacaacagaggatctctattttcagtccgacaatgctatcgcgatggctggggagggagaccaacaggacgcggcccataatatgggcaaccatcttccacttcttcctgccgagagtgaggaagaggacgagatggaagtggaggaccaggatagcaaggaggcaaaaaaaccaaacatcatcaacttcgatacttctctccccacatctcatacttacttgggggcagatatggaagaattccatggtcgcactctgcatgatgacgacagttgtcaggtgatcccagtgttgccccaggttatgatgattctgatcccaggccagactcttcctctccagttgttccatccccaagaggttagcatggtgcgaaacctcattcaaaaggaccgcacctttgccgtcttggcttacagtaatgtgcaggagcgcgaggcccagtttggaactaccgccgaaatatacgcctatagggaagaacaggacttcgggatcgaaattgtgaaggtgaaagccatcgggaggcagcggtttaaggttcttgaactcagaactcaatctgacgggattcagcaggcaaaggtacagatcctgcccgaatgtgtactgccatcaacaatgagcgcagttcagttggaatccctgaataagtgccagatctttcccagcaagccggtatcacgggaagaccagtgttcatacaaatggtggcagaagtatcagaagcgaaagtttcattgtgccaatctgacttcctggcctcgatggctgtacagtctgtacgatgctgaaactctcatggataggataaaaaagcagctgcgggagtgggacgagaacctcaaggatgacagtctgccatccaatcccatagatttttcttatcgtgtagccgcctgcctcccaattgatgatgtgttgcgcattcagttgctcaaaatcggctctgcgattcagcggctgcgatgcgagctcgacatcatgaacaagtgcactagtctgtgttgtaaacagtgtcaggaaaccgaaatcacaaccaagaacgaaatattttctcttagtctgtgcggacccatggccgcctatgtaaatccacacggttatgtacatgagacactcacagtctataaagcgtgtaatctgaatctcattgggcgcccaagtaccgagcatagctggttcccaggatatgcatggacagttgcccagtgcaaaatttgcgcctctcatatcgggtggaaatttaccgccacgaagaaggatatgagtcctcagaagttctggggcctcacacggagcgccttgttgcccactatccccgatactgaagatgagattagtccagataaggtgatcctgtgtctggtttga

SEQ ID NO: 20 - protein, 744 aa, synthetic construct
MPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQPSGEPTTEDLYFQSDNAIAMAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLV

SEQ ID NO: 21 - DNA, 1548 bp, synthetic construct
  source 1..1548: mol_type = other DNA
atgcctcagaaaccttgggacaaggatgcgtgggaaattcctcgcgaatccatcaagctcgtgaaaaggctgggagcagggcagttcggtgaggtttggatgggttattacaacaatagcacaaaagttgctgtgaaaaccctgaagcctggcacaatgtctgtgcaggcatttctggaggaggctaacctgatgaagactctgcagcacgacaagctggtgcggctctatgctgtcgttactcgggaggaacccatttatattatcacagagtatatggccaaaggctctcttcttgacttcttgaaatcagatgaggggggcaaggttctcctgcccaaattaatagacttttccgcccaaatagcagaaggcatggcctacattgagcgcaagaattatattcatcgcgatctcagggctgcgaacgtgcttgtgagtgagagcctgatgtgtaagatagctgatttcgggctggcaagggttatcgaggacaacgagtacacagcccgggaaggcgctaagttcccaatcaagtggaccgcaccagaagccattaactttgggtgttttacaattaagagtgacgtctggtcctttggtatacttctctacgaaattgtgacctacggaaagatcccctatcctggacggaccaacgcagacgtcatgactgcgttatcccagggctacagaatgccgcgcgtcgaaaactgtcccgatgagctctacgatatcatgaagatgtgttggaaggagaaggctgaggagcgccctaccttcgactatttacagtccgtcctggacgatttttacactgcaaccgaaggccaatatcagcaacagcctagcggcgagcccactacagaagatctttatttccagagtgacaacgccatcgctatgccacgccgagctgagaactgggacgaggcagaagttggcgccgaggaggccggcgtcgaggagtatggcccagaggaagatggcggtgaggagagtggagctgaggaaagcggccccgaggaaagtgggccagaagagctcggagcagaggaggaaatggaagctgggcggccacgtcccgttctgcgcagtgtcaatagtcgtgagcctagccaggtgatcttttgtaatagaagccctcgagtagtacttcccgtgtggttaaattttgacggcgaaccacagccttacccaaccttgccacctggaacaggtaggaggatccacagttacaggggtcacttgtggctgtttagagacgccggtactcacgacggcctgctcgtgaatcagacagaactttttgtcccttccctgaacgtggatggacaacctatcttcgcaaatatcaccctccccgtgtacaccctgaaggagagatgccttcaggtggtgagatctctggttaagcccgaaaactatcgtaggctggacatcgtgcgctctctttacgaggatctggaggaccacccaaatgttcaaaaggacctggagcgactgacccaggagcgcatagcgcatcagagaatgggagacgtgtag

SEQ ID NO: 22 - protein, 515 aa, synthetic construct
MPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQPSGEPTTEDLYFQSDNAIAMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDV

SEQ ID NO: 23 - RNA, 20 nt, synthetic construct
  source 1..20: mol_type = other RNA
gacagcgtgaacaggtagct